(19) United States
(12) Patent Application Publication
(10) Pub. No.: US 2026/0234224 A1
(43) Pub. Date: Aug. 13, 2026

(54) PEDIATRIC SARS-COV-2 ANTIBODIES AND USES THEREOF

(71) Applicant: VANDERBILT UNIVERSITY, Nashville, TN (US)

(72) Inventors: Steven Craig WALL, JR., Nashville, TN (US); Ivelin GEORGIEV, Nashville, TN (US)

(21) Appl. No.: 19/152,772

(22) PCT Filed: Feb. 2, 2024
(86) PCT No.: PCT/US2024/014323

§ 371 Date: Jul. 31, 2025

Related U.S. Application Data
(60) Provisional application No. 63/443,203, filed on Feb. 3, 2023.
(60) Provisional application No. 63/582,987, filed on Sep. 15, 2023.

(51) Int. Cl. C07K 16/104; A61P 31/14

(52) U.S. Cl. CPC C07K 16/104; A61P 31/14; C07K 2317/21; C07K 2317/565; C07K 2317/76; C07K 2317/92

(57) ABSTRACT

The present disclosure relates to antibodies and uses thereof for treating, preventing, and detecting coronavirus infection.

### CROSS REFERENCE TO RELATED APPLICATIONS

This application claims the benefit of U.S. Provisional Patent Application Ser. No. 63/443,203 filed Feb. 3, 2023, and U.S. Provisional Patent Application Ser. No. 63/582,987 filed Sep. 15, 2023, the disclosures of which are expressly incorporated herein by reference in their entireties.

### STATEMENT REGARDING FEDERALLY SPONSORED RESEARCH

This invention was made with government support under Grant No. R01 AI131722 and R01 AI175245 awarded by the National Institutes of Health. The government has certain rights in the invention.

### FIELD

The present disclosure relates to antibodies and uses thereof for treating, preventing, and detecting coronavirus infection.

### REFERENCE TO SEQUENCE LISTING

The Sequence Listing submitted Feb. 2, 2024 as an .XML file named “10644-157WO1_ST26.xml,” created on Feb. 2, 2024, and having a size of 2,693,321 bytes is hereby incorporated by reference pursuant to 37 C.F.R. § 1.52(e)(5).

### BACKGROUND

Neutralizing antibodies are associated with protection against SARS-CoV-2 and are therefore an important variable to consider when evaluating differences in protection. From the beginning of the COVID-19 pandemic, children have exhibited different susceptibility to SARS-CoV-2 infection, reinfection, and disease, compared to adults. Yet there is currently limited information about the types and specificities of neutralizing antibodies in children, with studies generally limited to assessment of polyclonal responses. What is needed are novel antibodies for treating viral infections (e.g., SARS-CoV-2) in pediatric and adult patients.

### SUMMARY

In accordance with the purposes of the disclosed materials and methods, as embodied and broadly described herein, the disclosed subject matter, in one aspect, relates to antibodies and uses thereof.

Thus, in one example, a recombinant antibody is provided, including a light chain variable region (VL) that comprises a light chain complementarity determining region (CDRL)1, a CDRL2, and a CDRL3 and a heavy chain variable region (VH) that comprises a heavy chain complementarity determining region (CDRH)1, CDRH2, CDRH3, wherein: CDRL3 comprises a sequence at least 60% identical to a sequence selected from SEQ ID NOs: 1948-2006; and CDRH3 comprises a sequence at least 60% identical to a sequence selected from SEQ ID NOs: 1771-1829.

In a further example, a nucleic acid encoding the recombinant antibody of a recombinant antibody as disclosed herein is provided.

Additionally, a recombinant expression cassette or plasmid is provided, including a sequence to express a recombinant antibody as disclosed herein.

Also, a cell is provided, including the expression cassette or plasmid disclosed herein.

Further, a method of treating a coronavirus infection in a subject is provided, including administering to the subject a therapeutically effective amount of a recombinant antibody disclosed herein.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DESCRIPTION OF DRAWINGS

The accompanying figures, which are incorporated in and constitute a part of this specification, illustrate several aspects described below.

FIGS. 1A-1E show pediatric antibody discovery using LIBRA-seq with a diverse CoV antigen panel. FIG. 1A shows 59 IgG and IgA cells identified by LIBRA-seq are shown as circles, with their respective LIBRA-seq scores (LSS) for: SARS-CoV-2 (x-axis), minimum score for the alpha and beta SARS-CoV-2 variants (y-axis), SARS-CoV (color heatmap), and average score for the zoonotic CoV spikes used in the LIBRA-seq library (size of circle). FIG. 1B shows recombinant antibodies were produced by microexpression and screened by ELISA against a panel of recombinant CoV spike antigens. For the antibodies that were positive for SARS-CoV-2 spike binding, shown are: (left) binding heatmap for each antibody (rows) against a panel of SARS-CoV-2 variants and other coronaviruses (columns), and (right) number of non-SARS-CoV-2 antigens (y-axis) that each antibody (x-axis) was able to recognize. FIG. 1C shows for each antibody (rows), the CDR amino acid sequences and lengths, V-gene and J-gene and % nucleotide identities, and isotype (columns). D,E, ELISA AUC values from purified monoclonal antibody binding to (FIG. 1D) SARS-CoV-2 variant spike proteins and (FIG. 1E) other betacoronavirus spike proteins are shown as heatmaps from minimum (white) to maximum (purple) binding.

FIGS. 2A-2B show pediatric antibody neutralization of SARS-CoV-2 variants. FIG. 2A shows SARS-CoV-2-VSV-G neutralization was performed for a panel of antibodies against SARS-CoV-2 VSV-G with the D614G mutation. FIG. 2B shows additional lentiviral pseudovirus neutralization assays were performed against SARS-CoV-2 D614G and omicron variants (BA.2, BA.4/5, BA.4.6, BA.2.75.2, BA.2.75+R346K, XBB, XBB.1.5, and BQ1.1). Data represent the percent neutralization or relative infection as mean±s.d. The IC₅₀ values calculated in GraphPad Prism software by four-parameter best-fit analysis are shown on the bottom right as a color heatmap from least (white) to most (purple) potent.

FIGS. 3A-3C show epitope and ACE2 blocking analysis of pediatric antibodies. FIG. 3A shows ELISA AUC values for recombinant SARS-CoV-2 spike antigen domains are shown as heatmaps from minimum (white) to maximum (purple) binding. FIG. 3B shows percent ACE2 blocking by ELISA for SARS-CoV-2 and SARS-CoV spike proteins and is depicted as a heatmap from 0% (white) to 100% (blue). FIG. 3C shows antibody-antibody competition ELISAs performed for antibodies 71281-31 and 71281-33 against other pediatric antibodies identified here, as well as previously published adult antibodies. Percent competition is shown as a heatmap from 0 percent (white) to 100 percent (purple). Non-biotinylated competitor antibodies were coated first and then biotinylated 71281-31 and 71281-33 were added to detect competition as described in more detail in Methods.

FIGS. 4A-4B show public clonotype analysis of SARS-CoV-2 pediatric antibodies. FIG. 4A shows for each pediatric antibody (separate plot), previously published adult antibodies (dots), with the respective CDRH3 (x-axis) and CDRL3 (y-axis) identity, colored according to V-gene usage: blue if both the VH and VL of the given pediatric and adult antibodies match, orange if only the VH match, purple if only the VL match, and grey if neither match but at least one of the CDRH3 and CDRL3 have >50% sequence identity for the pediatric vs. adult antibody. FIG. 4B shows for each pediatric antibody (column), the CDRH3 identity (values; heatmap) against a set of adult antibodies in clinical use or advanced preclinical development (rows). Cell border color corresponds to the V-gene usage described in (A).

FIGS. 5A-5B show sample information for groups 71281 and 71282. FIG. 5A shows samples from groups 71281 and 71282 broken down by age group and sex. FIG. 5B shows an age distribution for each group for males (gray), females (pink), and overall (teal) are on the left and a summary of the age distribution for samples pooled in each experimental group is on the right.

FIGS. 6A-6B shows ELISA curves for FIGS. 1D-1E and FIG. 3A. FIG. 6A shows antibodies tested for binding to a panel of CoV antigens by ELISA. The SARS-CoV-2/SARS-CoV cross reactive antibody CR3022 was used as a positive control. The influenza HA reactive antibody 3602-1707 was used as a negative control. FIG. 6B shows antibodies tested for binding to different subdomains of the SARS-CoV-2 spike glycoprotein (RBD, NTD, S1, and S2). The positive control for RBD and S1 was CR3022, the positive control for NTD was a known NTD binding antibody 5317-9, and the positive control for the S2 domain was the S2-reactive antibody 54043-5. ELISAs were performed in technical duplicates with at least 2 biological duplicates. Data are represented as means±SEMs.

FIG. 7A-7B shows additional neutralization data for the pediatric SARS-CoV-2 antibodies. FIG. 7A shows VSV-G SARS-CoV-2 neutralization screening results for pediatric antibodies at two concentrations (0.5 μg/mL and 5 μg/mL). All antibodies that had a phenotype represented by the negative control rDENV-2D22 in panel B were considered negative and are denoted as a red X. All antibodies that had a phenotype represented by the positive neutralizing control, S309, were considered positive and denoted with a green check mark. FIG. 7B shows VSV-G SARS-CoV neutralization screenings were performed for antibody 71281-31 at two concentrations (0.5 μg/mL and 5 μg/mL). The data for 71281-31 is shown on the first row, positive control S309 on the second row, and negative control rDENV-2D22 on the 3^{rd} in triplicate. For each plot, the X axis represents time from 0-72 hours, and the Y axis represents the normalized cell index.

FIG. 8 shows public antibody analysis for non-neutralizing SARS-CoV-2 spike-binding antibodies. For each pediatric antibody (separate plot), shown are previously published adult antibodies (dots), with the respective CDRH3 (x-axis) and CDRL3 (y-axis) identity, colored according to V-gene usage: blue if both the VH and VL of the given pediatric and adult antibodies match, orange if only the VH match, purple if only the VL match, and grey if neither match but at least one of the CDRH3 and CDRL3 have >50% sequence identity for the pediatric vs. adult antibody.

FIGS. 9A-9C shows nsEM of SARS-CoV-2 spike protein in complex with Fab 71281-33. FIG. 9A shows images of 2D classes of the complex. Box size 128 pixel (4.36 A/pix). FIG. 9B shows a 3D map/model of spike (PDB 7XIW) complex with 2 Fab (PDB 12E8) (top and side view). One RBD is in the up position and the second is in the down position. Map color gray for spike and magenta for Fab. FIG. 9C shows a 3D map/model of spike (PDB 7XIW) complex with 3 Fab (PDB 12E8) (top and side view). Two RBDs are in the up position and one is in the down position. Map color is gray for spike and magenta for Fab.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Terminology

The term “about” as used herein when referring to a measurable value such as an amount, a percentage, and the like, is meant to encompass variations of +20%, +10%, +5%, or ±1% from the measurable value.

“Administration” to a subject or “administering” includes any route of introducing or delivering to a subject an agent. Administration can be carried out by any suitable route, including oral, intravenous, intraperitoneal, intranasal, inhalation and the like. Administration includes self-administration and the administration by another.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, the term “subject” or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

As used herein, the term “antigen” refers to a molecule that is capable of binding to an antibody. In some embodiments, the antigen stimulates an immune response such as by production of antibodies specific for the antigen.

In the present invention, “specific for” and “specificity” means a condition where one of the molecules is involved in selective binding. Accordingly, an antibody that is specific for one antigen selectively binds that antigen and not other antigens.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof. The antibodies can be tested for their desired activity using the in vitro assays described herein, or by analogous methods, after which their in vivo therapeutic and/or prophylactic activities are tested according to known clinical testing methods. Native antibodies are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

Each antibody molecule is made up of the protein products of two genes: heavy-chain gene and light-chain gene. The heavy-chain gene is constructed through somatic recombination of V, D, and J gene segments. In human, there are 51 VH, 27 DH, 6 JH, 9 CH gene segments on human chromosome 14. The light-chain gene is constructed through somatic recombination of V and J gene segments. There are 40 Vκ, 31 Vλ, 5 Jκ, 4 Jλ gene segments on human chromosome 14 (80 VJ). The heavy-chain constant domains that correspond to the different classes of immunoglobulins are called u, 6, s, y, and p, respectively. The “light chains” of antibodies from any vertebrate species can be assigned to one of two clearly distinct types, called kappa (κ) and lambda (λ), based on the amino acid sequences of their constant domains.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods. DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or antigen binding fragment thereof” or “antibody or fragments thereof” encompasses chimeric antibodies and hybrid antibodies, with dual or multiple antigen or epitope specificities, and fragments, such as F(ab′)2, Fab′, Fab, Fv, sFv, scFv, nanoantibody and the like, including hybrid fragments. Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

The terms “antigen binding site”, “binding site” and “binding domain” refer to the specific elements, parts or amino acid residues of a polypeptide, such as an antibody, that bind the antigenic determinant or epitope.

An “antibody heavy chain,” as used herein, refers to the larger of the two types of polypeptide chains present in all antibody molecules in their naturally occurring conformations.

An “antibody light chain,” as used herein, refers to the smaller of the two types of polypeptide chains present in all antibody molecules in their naturally occurring conformations, κ and λ light chains refer to the two major antibody light chain isotypes.

The term “CDR” as used herein refers to the “complementarity determining regions” of the antibody which consist of the antigen binding loops. (Kabat E. A. et al., (1991) Sequences of proteins of immunological interest. NIH Publication 91-3242). Each of the two variable domains of an antibody Fv fragment contain, for example, three CDRs.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen-binding regions. The amino acid sequence boundaries of a CDR can be determined by one of skill in the art using any of a number of known numbering schemes, including those described by Kabat et al., supra (“Kabat” numbering scheme): Al-Lazikani et al., 1997. J. Mol. Biol., 273:927-948 (“Chothia” numbering scheme); MacCallum et al., 1996, J. Mol. Biol, 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme); each of which is incorporated by reference in its entirety.

“Effective amount” encompasses, without limitation, an amount that can ameliorate, reverse, mitigate, prevent, or diagnose a symptom or sign of a medical condition or disorder. Unless dictated otherwise, explicitly or by context, an “effective amount” is not limited to a minimal amount sufficient to ameliorate a condition. The severity of a disease or disorder, as well as the ability of a treatment to prevent, treat, or mitigate, the disease or disorder can be measured, without implying any limitation, by a biomarker or by a clinical parameter. In some embodiments, the term “effective amount of a recombinant antibody” refers to an amount of a recombinant antibody sufficient to prevent, treat, or mitigate a coronavirus infection (e.g., SARS-CoV-2 infection).

The “fragments” or “functional fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the functional fragment must possess a bioactive property, such as binding to a coronavirus antigen (e.g., SARS-CoV-2 antigen), and/or ameliorating the viral infection.

The term “identity” or “homology” shall be construed to mean the percentage of nucleotide bases or amino acid residues in the candidate sequence that are identical with the bases or residues of a corresponding sequence to which it is compared, after aligning the sequences and introducing gaps, if necessary to achieve the maximum percent identity for the entire sequence, and not considering any conservative substitutions as part of the sequence identity. A polynucleotide or polynucleotide region (or a polypeptide or polypeptide region) that has a certain percentage (for example, 80%, 85%, 90%, or 95%) of “sequence identity” to another sequence means that, when aligned, that percentage of bases (or amino acids) are the same in comparing the two sequences. This alignment and the percent homology or sequence identity can be determined using software programs known in the art. Such alignment can be provided using, for instance, the method of Needleman et al. (1970) J. Mol. Biol. 48: 443-453, implemented conveniently by computer programs such as the Align program (DNAstar, Inc.).

The term “increased” or “increase” as used herein generally means an increase by a statically significant amount; for example, “increased” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the terms “nanobody”, “V_{H}H”, “V_{H}H antibody fragment” and “single domain antibody” are used indifferently and designate a variable domain of a single heavy chain of an antibody of the type found in Camelidae, which are without any light chains, such as those derived from Camelids as described in PCT Publication No. WO 94/04678, which is incorporated by reference in its entirety.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

“Nucleotide,” “nucleoside,” “nucleotide residue,” and “nucleoside residue,” as used herein, can mean a deoxyribonucleotide, ribonucleotide residue, or another similar nucleoside analogue. A nucleotide is a molecule that contains a base moiety, a sugar moiety and a phosphate moiety.

Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

The method and the system disclosed here including the use of primers, which are capable of interacting with the disclosed nucleic acids, such as the antigen barcode as disclosed herein. In certain embodiments the primers are used to support DNA amplification reactions. Typically, the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically, the disclosed primers hybridize with the disclosed nucleic acids or region of the nucleic acids or they hybridize with the complement of the nucleic acids or complement of a region of the nucleic acids.

The term “amplification” refers to the production of one or more copies of a genetic fragment or target sequence, specifically the “amplicon”. As it refers to the product of an amplification reaction, amplicon is used interchangeably with common laboratory terms, such as “PCR product.”

The term “polypeptide” refers to a compound made up of a single chain of D- or L-amino acids or a mixture of D- and L-amino acids joined by peptide bonds.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA.

An “expression cassette” refers to a DNA coding sequence or segment of DNA that code for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”.

Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together. The term “vector” as used herein includes autonomously replicating nucleotide sequences as well as genome integrating nucleotide sequences. A common type of vector is a “plasmid”, which generally is a self-contained molecule of double-stranded DNA that can readily accept additional (foreign) DNA and which can readily be introduced into a suitable host cell. A plasmid vector often contains coding DNA and promoter DNA and has one or more restriction sites suitable for inserting foreign DNA. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

The term “host cell” as used herein shall refer to primary subject cells trans-formed to produce a particular recombinant protein, such as an antibody as described herein, and any progeny thereof. It should be understood that not all progeny are exactly identical to the parental cell (due to deliberate or inadvertent mutations or differences in environment), however, such altered progeny are included in these terms, so long as the progeny retain the same functionality as that of the originally transformed cell. The term “host cell line” refers to a cell line of host cells as used for expressing a recombinant gene to produce recombinant polypeptides such as recombinant antibodies. The term “cell line” as used herein refers to an established clone of a particular cell type that has acquired the ability to proliferate over a prolonged period of time. Such host cell or host cell line may be maintained in cell culture and/or cultivated to produce a recombinant polypeptide.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof. The term “gene” or “gene sequence” includes, for example, control sequences upstream of the coding sequence.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “specificity” refers to the number of different types of antigens or antigenic determinants to which a particular antigen-binding molecule (such as the recombinant antibody of the invention) can bind. As used herein, the term “specifically binds,” as used herein with respect to a recombinant antibody refers to the recombinant antibody's preferential binding to one or more epitopes as compared with other epitopes. Specific binding can depend upon binding affinity and the stringency of the conditions under which the binding is conducted. In one example, an antibody specifically binds an epitope when there is high affinity binding under stringent conditions.

It should be understood that the specificity of an antigen-binding molecule (e.g., the recombinant antibodies of the present invention) can be determined based on affinity and/or avidity. The affinity, represented by the equilibrium constant for the dissociation of an antigen with an antigen-binding molecule (K_{D}), is a measure for the binding strength between an antigenic determinant and an antigen-binding site on the antigen-binding molecule: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as the recombinant antibodies of the present invention) and the pertinent antigen. Avidity is related to both the affinity between an antigenic determinant and its antigen binding site on the antigen-binding molecule and the number of pertinent binding sites present on the antigen-binding molecule. Typically, antigen-binding proteins (such as the recombinant antibodies of the invention) will bind to their antigen with a dissociation constant (K_{D}) of 10⁻⁵ to 10⁻¹² moles/liter or less, and preferably 10⁻⁷ to 10⁻¹² moles/liter or less, and more preferably 10⁻⁸ to 10⁻¹² moles/liter.

“Therapeutically effective amount” refers to the amount of a composition such as recombinant antibody that will elicit the biological or medical response of a tissue, system, animal, or human that is being sought by the researcher, veterinarian, medical doctor or other clinician over a generalized period of time. In some embodiments, a desired response is reduction of coronaviral titers in a subject. In some embodiments, the desired response is mitigation of coronavirus infection and/or related symptoms. In some instances, a desired biological or medical response is achieved following administration of multiple dosages of the composition to the subject over a period of days, weeks, or years. The therapeutically effective amount will vary depending on the composition, the disorder or conditions and its severity, the route of administration, time of administration, rate of excretion, drug combination, judgment of the treating physician, dosage form, and the age, weight, general health, sex and/or diet of the subject to be treated. The therapeutically effective amount of recombinant antibodies as described herein can be determined by one of ordinary skill in the art.

A therapeutically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a biological marker, such as decreased viral titers, decreased viral RNA levels, increase in CD4 T lymphocyte counts, and/or prolonged survival of a subject. It will be understood, that the total daily usage of the compositions and formulations as disclosed herein will be decided by the attending physician within the scope of sound medical judgment. The exact amount required will vary depending on factors such as the type of disease being treated.

The terms “treat,” “treating,” “treatment,” and grammatical variations thereof as used herein, include partially or completely delaying, alleviating, mitigating or reducing the intensity of one or more attendant symptoms. Treatments according to the invention may be applied preventively, prophylactically, palliatively or remedially. Prophylactic treatments are administered to a subject prior to onset (e.g., before obvious signs of an infection), during early onset (e.g., upon initial signs and symptoms of an infection), after an established development of an infection, or during chronic infection. Prophylactic administration can occur for several minutes to months prior to the manifestation of an infection.

As used herein, the term “preventing” a disorder or unwanted physiological event in a subject refers specifically to the prevention of the occurrence of symptoms and/or their underlying cause, wherein the subject may or may not exhibit heightened susceptibility to the disorder or event.

### Antibodies and Compositions

Provided herein is a recombinant antibody comprising a light chain variable region (VL) that comprises a light chain complementarity determining region (CDRL)1, a CDRL2, and a CDRL3 and a heavy chain variable region (VH) that comprises a heavy chain complementarity determining region (CDRH)1, CDRH2, CDRH3, wherein: CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NOs: 1948-2006; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NOs: 1771-1829.

In some examples, CDRL3 comprises at least one amino acid substitution when compared to SEQ ID NOs: 1948-2006. In some examples, CDRL3 comprises at least 1, 2, 3, 4, 5, or 6 substitutions when compared to SEQ ID NOs: 1948-2006.

In further examples, CDRH3 comprises at least one amino acid substitution when compared to SEQ ID NOs: 1771-1829. In further examples, CDRH3 comprises at least 1, 2, 3, 4, 5, or 6 substitutions when compared to SEQ ID NOs: 1771-1829.

In certain examples, CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NOs: 1889-1947; and CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NOs: 1712-1770.

In specific examples, CDRL2 comprises at least one amino acid substitution when compared to SEQ ID NOs: 1889-1947. In specific examples, CDRL2 comprises at least 1, 2, 3, 4, 5, or 6 substitutions when compared to SEQ ID NOs: 1889-1947.

In some examples, CDRH2 comprises at least one amino acid substitution when compared to SEQ ID NOs: 1712-1770. In some examples, CDRH2 comprises at least 1, 2, 3, 4, 5, or 6 substitutions when compared to SEQ ID NOs: 1712-1770.

In further examples, CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NOs: 1830-1888; and CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NOs: 1653-1711.

In certain examples, CDRL1 comprises at least one amino acid substitution when compared to SEQ ID NOs: 1830-1888. In certain examples, CDRL1 comprises at least 1, 2, 3, 4, 5, or 6 substitutions when compared to SEQ ID NOs: 1830-1888.

In specific examples, CDRH1 comprises at least one amino acid substitution when compared to SEQ ID NOs: 1653-1711. In specific examples, CDRH1 comprises at least 1, 2, 3, 4, 5, or 6 substitutions when compared to SEQ ID NOs: 1653-1711.

In some examples, VH comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NOs: 178-236.

In further examples, VL comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NOs: 1004-1062.

In certain examples, CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NO: 1950, SEQ ID NO: 1953, SEQ ID NO: 1967, SEQ ID NO: 1969, SEQ ID NO: 1973, SEQ ID NO: 1978, SEQ ID NO: 1979, SEQ ID NO: 1980, SEQ ID NO: 1982, SEQ ID NO: 1983, SEQ ID NO: 1984, SEQ ID NO: 1990, SEQ ID NO: 1992, SEQ ID NO: 1993, SEQ ID NO: 1996, SEQ ID NO: 2000, or SEQ ID NO: 2004; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NO: 1773, SEQ ID NO: 1776, SEQ ID NO: 1790, SEQ ID NO: 1792, SEQ ID NO: 1796, SEQ ID NO: 1801, SEQ ID NO: 1802, SEQ ID NO: 1803, SEQ ID NO: 1805, SEQ ID NO: 1806, SEQ ID NO: 1807, SEQ ID NO: 1813, SEQ ID NO: 1815, SEQ ID NO: 1816, SEQ ID NO: 1819, SEQ ID NO: 1823, or SEQ ID NO: 1827.

In specific examples, CDRL3 comprises at least one amino acid substitution when compared to SEQ ID NO: 1950, SEQ ID NO: 1953, SEQ ID NO: 1967, SEQ ID NO: 1969, SEQ ID NO: 1973, SEQ ID NO: 1978, SEQ ID NO: 1979, SEQ ID NO: 1980, SEQ ID NO: 1982, SEQ ID NO: 1983, SEQ ID NO: 1984, SEQ ID NO: 1990, SEQ ID NO: 1992, SEQ ID NO: 1993, SEQ ID NO: 1996, SEQ ID NO: 2000, or SEQ ID NO: 2004. In specific examples, CDRL3 comprises at least 1, 2, 3, 4, 5, or 6 substitutions when compared to SEQ ID NO: 1950, SEQ ID NO: 1953, SEQ ID NO: 1967, SEQ ID NO: 1969, SEQ ID NO: 1973, SEQ ID NO: 1978, SEQ ID NO: 1979, SEQ ID NO: 1980, SEQ ID NO: 1982, SEQ ID NO: 1983, SEQ ID NO: 1984, SEQ ID NO: 1990, SEQ ID NO: 1992, SEQ ID NO: 1993, SEQ ID NO: 1996, SEQ ID NO: 2000, or SEQ ID NO: 2004.

In some examples, CDRH3 comprises at least one amino acid substitution when compared to SEQ ID NO: 1773, SEQ ID NO: 1776, SEQ ID NO: 1790, SEQ ID NO: 1792, SEQ ID NO: 1796, SEQ ID NO: 1801, SEQ ID NO: 1802, SEQ ID NO: 1803, SEQ ID NO: 1805, SEQ ID NO: 1806, SEQ ID NO: 1807, SEQ ID NO: 1813, SEQ ID NO: 1815, SEQ ID NO: 1816, SEQ ID NO: 1819, SEQ ID NO: 1823, or SEQ ID NO: 1827. In some examples, CDRH3 comprises at least 1, 2, 3, 4, 5, or 6 substitutions when compared to SEQ ID NO: 1773, SEQ ID NO: 1776, SEQ ID NO: 1790, SEQ ID NO: 1792, SEQ ID NO: 1796, SEQ ID NO: 1801, SEQ ID NO: 1802, SEQ ID NO: 1803, SEQ ID NO: 1805, SEQ ID NO: 1806, SEQ ID NO: 1807, SEQ ID NO: 1813, SEQ ID NO: 1815, SEQ ID NO: 1816, SEQ ID NO: 1819, SEQ ID NO: 1823, or SEQ ID NO: 1827.

In further examples, CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NO: 1891, SEQ ID NO: 1894, SEQ ID NO: 1908, SEQ ID NO: 1910, SEQ ID NO: 1914, SEQ ID NO: 1919, SEQ ID NO: 1920, SEQ ID NO: 1921, SEQ ID NO: 1923, SEQ ID NO: 1924, SEQ ID NO: 1925, SEQ ID NO: 1931, SEQ ID NO: 1933, SEQ ID NO: 1934, SEQ ID NO: 1937, SEQ ID NO: 1941, or SEQ ID NO: 1945; and CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NO: 1714, SEQ ID NO: 1717, SEQ ID NO: 1731, SEQ ID NO: 1733, SEQ ID NO: 1737, SEQ ID NO: 1742, SEQ ID NO: 1743, SEQ ID NO: 1744, SEQ ID NO: 1746, SEQ ID NO: 1747, SEQ ID NO: 1748, SEQ ID NO: 1754, SEQ ID NO: 1756, SEQ ID NO: 1757, SEQ ID NO: 1760, SEQ ID NO: 1764, or SEQ ID NO: 1768.

In certain examples, CDRL2 comprises at least one amino acid substitution when compared to SEQ ID NO: 1891, SEQ ID NO: 1894, SEQ ID NO: 1908, SEQ ID NO: 1910, SEQ ID NO: 1914, SEQ ID NO: 1919, SEQ ID NO: 1920, SEQ ID NO: 1921, SEQ ID NO: 1923, SEQ ID NO: 1924, SEQ ID NO: 1925, SEQ ID NO: 1931, SEQ ID NO: 1933, SEQ ID NO: 1934, SEQ ID NO: 1937, SEQ ID NO: 1941, or SEQ ID NO: 1945. In certain examples, CDRL2 comprises at least 1, 2, 3, 4, 5, or 6 substitutions when compared to SEQ ID NO: 1891, SEQ ID NO: 1894, SEQ ID NO: 1908, SEQ ID NO: 1910, SEQ ID NO: 1914, SEQ ID NO: 1919, SEQ ID NO: 1920, SEQ ID NO: 1921, SEQ ID NO: 1923, SEQ ID NO: 1924, SEQ ID NO: 1925, SEQ ID NO: 1931, SEQ ID NO: 1933, SEQ ID NO: 1934, SEQ ID NO: 1937, SEQ ID NO: 1941, or SEQ ID NO: 1945.

In specific examples, CDRH2 comprises at least one amino acid substitution when compared to SEQ ID NO: 1714, SEQ ID NO: 1717, SEQ ID NO: 1731, SEQ ID NO: 1733, SEQ ID NO: 1737, SEQ ID NO: 1742, SEQ ID NO: 1743, SEQ ID NO: 1744, SEQ ID NO: 1746, SEQ ID NO: 1747, SEQ ID NO: 1748, SEQ ID NO: 1754, SEQ ID NO: 1756, SEQ ID NO: 1757, SEQ ID NO: 1760, SEQ ID NO: 1764, or SEQ ID NO: 1768. In specific examples, CDRH2 comprises at least 1, 2, 3, 4, 5, or 6 substitutions when compared to SEQ ID NO: 1714, SEQ ID NO: 1717, SEQ ID NO: 1731, SEQ ID NO: 1733, SEQ ID NO: 1737, SEQ ID NO: 1742, SEQ ID NO: 1743, SEQ ID NO: 1744, SEQ ID NO: 1746, SEQ ID NO: 1747, SEQ ID NO: 1748, SEQ ID NO: 1754, SEQ ID NO: 1756, SEQ ID NO: 1757, SEQ ID NO: 1760, SEQ ID NO: 1764, or SEQ ID NO: 1768.

In some examples, CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NO: 1832, SEQ ID NO: 1835, SEQ ID NO: 1849, SEQ ID NO: 1851, SEQ ID NO: 1855, SEQ ID NO: 1860, SEQ ID NO: 1861, SEQ ID NO: 1862, SEQ ID NO: 1864, SEQ ID NO: 1865, SEQ ID NO: 1866, SEQ ID NO: 1872, SEQ ID NO: 1874, SEQ ID NO: 1875, SEQ ID NO: 1878, SEQ ID NO: 1882, or SEQ ID NO: 1886; and CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NO: 1655, SEQ ID NO: 1658, SEQ ID NO: 1672, SEQ ID NO: 1674, SEQ ID NO: 1678, SEQ ID NO: 1683, SEQ ID NO: 1684, SEQ ID NO: 1685, SEQ ID NO: 1687, SEQ ID NO: 1688, SEQ ID NO: 1689, SEQ ID NO: 1695, SEQ ID NO: 1697, SEQ ID NO: 1698, SEQ ID NO: 1701, SEQ ID NO: 1705, or SEQ ID NO: 1709.

In further examples, CDRL1 comprises at least one amino acid substitution when compared to SEQ ID NO: 1832, SEQ ID NO: 1835, SEQ ID NO: 1849, SEQ ID NO: 1851, SEQ ID NO: 1855, SEQ ID NO: 1860, SEQ ID NO: 1861, SEQ ID NO: 1862, SEQ ID NO: 1864, SEQ ID NO: 1865, SEQ ID NO: 1866, SEQ ID NO: 1872, SEQ ID NO: 1874, SEQ ID NO: 1875, SEQ ID NO: 1878, SEQ ID NO: 1882, or SEQ ID NO: 1886. In further examples, CDRL1 comprises at least 1, 2, 3, 4, 5, or 6 substitutions when compared to SEQ ID NO: 1832, SEQ ID NO: 1835, SEQ ID NO: 1849, SEQ ID NO: 1851, SEQ ID NO: 1855, SEQ ID NO: 1860, SEQ ID NO: 1861, SEQ ID NO: 1862, SEQ ID NO: 1864, SEQ ID NO: 1865, SEQ ID NO: 1866, SEQ ID NO: 1872, SEQ ID NO: 1874, SEQ ID NO: 1875, SEQ ID NO: 1878, SEQ ID NO: 1882, or SEQ ID NO: 1886.

In certain examples, CDRH1 comprises at least one amino acid substitution when compared to SEQ ID NO: 1655, SEQ ID NO: 1658, SEQ ID NO: 1672, SEQ ID NO: 1674, SEQ ID NO: 1678, SEQ ID NO: 1683, SEQ ID NO: 1684, SEQ ID NO: 1685, SEQ ID NO: 1687, SEQ ID NO: 1688, SEQ ID NO: 1689, SEQ ID NO: 1695, SEQ ID NO: 1697, SEQ ID NO: 1698, SEQ ID NO: 1701, SEQ ID NO: 1705, or SEQ ID NO: 1709. In certain examples, CDRH1 comprises at least 1, 2, 3, 4, 5, or 6 substitutions when compared to SEQ ID NO: 1655, SEQ ID NO: 1658, SEQ ID NO: 1672, SEQ ID NO: 1674, SEQ ID NO: 1678, SEQ ID NO: 1683, SEQ ID NO: 1684, SEQ ID NO: 1685, SEQ ID NO: 1687, SEQ ID NO: 1688, SEQ ID NO: 1689, SEQ ID NO: 1695, SEQ ID NO: 1697, SEQ ID NO: 1698, SEQ ID NO: 1701, SEQ ID NO: 1705, or SEQ ID NO: 1709.

In specific examples, VH comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NO: 180, SEQ ID NO: 183, SEQ ID NO: 197, SEQ ID NO: 199, SEQ ID NO: 203, SEQ ID NO: 208, SEQ ID NO: 209, SEQ ID NO: 210, SEQ ID NO: 212, SEQ ID NO: 213, SEQ ID NO: 214, SEQ ID NO: 220, SEQ ID NO: 222, SEQ ID NO: 223, SEQ ID NO: 226, SEQ ID NO: 230, or SEQ ID NO: 234.

In some examples, VL comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to a sequence selected from SEQ ID NO: 1006, SEQ ID NO: 1009, SEQ ID NO: 1023, SEQ ID NO: 1025, SEQ ID NO: 1029, SEQ ID NO: 1034, SEQ ID NO: 1035, SEQ ID NO: 1036, SEQ ID NO: 1038, SEQ ID NO: 1039, SEQ ID NO: 1040, SEQ ID NO: 1046, SEQ ID NO: 1048, SEQ ID NO: 1049, SEQ ID NO: 1052, SEQ ID NO: 1056, or SEQ ID NO: 1060.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1832; CDRL2 is SEQ ID NO: 1891; CRDL3 is SEQ ID NO: 1950; CRDH1 is SEQ ID NO: 1655; CRDH2 is SEQ ID NO: 1714; and CDRH3 is SEQ ID NO: 1773.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1832; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1891; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1950; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1655; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1714; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1773.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1835; CDRL2 is SEQ ID NO: 1894; CRDL3 is SEQ ID NO: 1953; CRDH1 is SEQ ID NO: 1658; CRDH2 is SEQ ID NO: 1717; and CDRH3 is SEQ ID NO: 1776.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1835; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1894; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1953; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1658; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1717; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1776.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1849; CDRL2 is SEQ ID NO: 1908; CRDL3 is SEQ ID NO: 1967; CRDH1 is SEQ ID NO: 1672; CRDH2 is SEQ ID NO: 1731; and CDRH3 is SEQ ID NO: 1790.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1849; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1908; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1967; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1672; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1731; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1790.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1851; CDRL2 is SEQ ID NO: 1910; CRDL3 is SEQ ID NO: 1969; CRDH1 is SEQ ID NO: 1674; CRDH2 is SEQ ID NO: 1733; and CDRH3 is SEQ ID NO: 1792.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1851; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1910; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1969; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1674; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1733; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1792.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1855; CDRL2 is SEQ ID NO: 1914; CRDL3 is SEQ ID NO: 1973; CRDH1 is SEQ ID NO: 1678; CRDH2 is SEQ ID NO: 1737; and CDRH3 is SEQ ID NO: 1796.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1855; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1914; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1973; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1678; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1737; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1796.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1860; CDRL2 is SEQ ID NO: 1919; CRDL3 is SEQ ID NO: 1978; CRDH1 is SEQ ID NO: 1683; CRDH2 is SEQ ID NO: 1742; and CDRH3 is SEQ ID NO: 1801.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1860; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1919; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1978; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1683; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1742; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1801.

In specific examples, VH comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 208.

In specific examples, VL comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1034.

In specific examples, CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1860.

In specific examples, CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1919.

In specific examples, CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1978.

In certain examples, the antibody comprises a light chain complementarity determining region CDRL1, CDRL2, and CDRL3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1860; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1919; and CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1978.

In specific examples, CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1683.

In specific examples, CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1742.

In specific examples, CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1801.

In certain examples, the antibody comprises a heavy chain complementarity determining region CDRH1, CDRH2, and CDRH3, wherein: CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1683; CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1742; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1801.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1861; CDRL2 is SEQ ID NO: 1920; CRDL3 is SEQ ID NO: 1979; CRDH1 is SEQ ID NO: 1684; CRDH2 is SEQ ID NO: 1743; and CDRH3 is SEQ ID NO: 1802.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1861; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1920; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1979; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1684; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1743; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1802.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1862; CDRL2 is SEQ ID NO: 1921; CRDL3 is SEQ ID NO: 1980; CRDH1 is SEQ ID NO: 1685; CRDH2 is SEQ ID NO: 1744; and CDRH3 is SEQ ID NO: 1803.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1862; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1921; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1980; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1685; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1744; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1803.

In specific examples, VH comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 210.

In specific examples, VL comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1036.

In specific examples, CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1862.

In specific examples, CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1921.

In specific examples, CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1980.

In certain examples, the antibody comprises a light chain complementarity determining region CDRL1, CDRL2, and CDRL3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1862; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1921; and CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1980.

In specific examples, CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1685.

In specific examples, CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1744.

In specific examples, CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1803.

In certain examples, the antibody comprises a heavy chain complementarity determining region CDRH1, CDRH2, and CDRH3, wherein: CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1685; CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1744; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1803.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1864; CDRL2 is SEQ ID NO: 1923; CRDL3 is SEQ ID NO: 1982; CRDH1 is SEQ ID NO: 1687; CRDH2 is SEQ ID NO: 1746; and CDRH3 is SEQ ID NO: 1805.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1864; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1923; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1982; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1687; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1746; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1805.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1865; CDRL2 is SEQ ID NO: 1924; CRDL3 is SEQ ID NO: 1983; CRDH1 is SEQ ID NO: 1688; CRDH2 is SEQ ID NO: 1747; and CDRH3 is SEQ ID NO: 1806.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1865; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1924; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1983; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1688; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1747; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1806.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1866; CDRL2 is SEQ ID NO: 1925; CRDL3 is SEQ ID NO: 1984; CRDH1 is SEQ ID NO: 1689; CRDH2 is SEQ ID NO: 1748; and CDRH3 is SEQ ID NO: 1807.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1866; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1925; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1984; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1689; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1748; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1807.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1872; CDRL2 is SEQ ID NO: 1931; CRDL3 is SEQ ID NO: 1990; CRDH1 is SEQ ID NO: 1695; CRDH2 is SEQ ID NO: 1754; and CDRH3 is SEQ ID NO: 1813.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1872; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1931; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1990; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1695; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1754; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1813.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1874; CDRL2 is SEQ ID NO: 1933; CRDL3 is SEQ ID NO: 1992; CRDH1 is SEQ ID NO: 1697; CRDH2 is SEQ ID NO: 1756; and CDRH3 is SEQ ID NO: 1815.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1874; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1933; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1992; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1697; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1756; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1815.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1875; CDRL2 is SEQ ID NO: 1934; CRDL3 is SEQ ID NO: 1993; CRDH1 is SEQ ID NO: 1698; CRDH2 is SEQ ID NO: 1757; and CDRH3 is SEQ ID NO: 1816.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1875; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1934; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1993; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1698; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1757; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1816.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1878; CDRL2 is SEQ ID NO: 1937; CRDL3 is SEQ ID NO: 1996; CRDH1 is SEQ ID NO: 1701; CRDH2 is SEQ ID NO: 1760; and CDRH3 is SEQ ID NO: 1819.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1878; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1937; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1996; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1701; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1760; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1819.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1882; CDRL2 is SEQ ID NO: 1941; CRDL3 is SEQ ID NO: 2000; CRDH1 is SEQ ID NO: 1705; CRDH2 is SEQ ID NO: 1764; and CDRH3 is SEQ ID NO: 1823.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1882; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1941; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 2000; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1705; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1764; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1823.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1886; CDRL2 is SEQ ID NO: 1945; CRDL3 is SEQ ID NO: 2004; CRDH1 is SEQ ID NO: 1709; CRDH2 is SEQ ID NO: 1768; and CDRH3 is SEQ ID NO: 1827.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1886; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1945; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 2004; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1709; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1768; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1827.

Further provided herein is a nucleic acid encoding the recombinant antibody as disclosed herein.

Also provide herein is a recombinant expression cassette or plasmid comprising a sequence to express a recombinant antibody disclosed herein.

Further provided herein is a host cell comprising an expression cassette or plasmid as disclosed herein.

### Methods

Further provided herein is a method of treating a coronavirus infection in a subject, comprising administering to the subject a therapeutically effective amount of the recombinant antibody disclosed herein.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1832; CDRL2 is SEQ ID NO: 1891; CRDL3 is SEQ ID NO: 1950; CRDH1 is SEQ ID NO: 1655; CRDH2 is SEQ ID NO: 1714; and CDRH3 is SEQ ID NO: 1773.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1832; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1891; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1950; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1655; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1714; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1773.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1835; CDRL2 is SEQ ID NO: 1894; CRDL3 is SEQ ID NO: 1953; CRDH1 is SEQ ID NO: 1658; CRDH2 is SEQ ID NO: 1717; and CDRH3 is SEQ ID NO: 1776.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1835; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1894; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1953; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1658; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1717; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1776.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1849; CDRL2 is SEQ ID NO: 1908; CRDL3 is SEQ ID NO: 1967; CRDH1 is SEQ ID NO: 1672; CRDH2 is SEQ ID NO: 1731; and CDRH3 is SEQ ID NO: 1790.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1849; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1908; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1967; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1672; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1731; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1790.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1851; CDRL2 is SEQ ID NO: 1910; CRDL3 is SEQ ID NO: 1969; CRDH1 is SEQ ID NO: 1674; CRDH2 is SEQ ID NO: 1733; and CDRH3 is SEQ ID NO: 1792.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1851; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1910; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1969; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1674; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1733; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1792.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1855; CDRL2 is SEQ ID NO: 1914; CRDL3 is SEQ ID NO: 1973; CRDH1 is SEQ ID NO: 1678; CRDH2 is SEQ ID NO: 1737; and CDRH3 is SEQ ID NO: 1796.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1855; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1914; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1973; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1678; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1737; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1796.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1860; CDRL2 is SEQ ID NO: 1919; CRDL3 is SEQ ID NO: 1978; CRDH1 is SEQ ID NO: 1683; CRDH2 is SEQ ID NO: 1742; and CDRH3 is SEQ ID NO: 1801.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1860; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1919; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1978; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1683; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1742; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1801.

In specific examples, VH comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 208.

In specific examples, VL comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1034.

In specific examples, CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1860.

In specific examples, CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1919.

In specific examples, CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1978.

In certain examples, the antibody comprises a light chain complementarity determining region CDRL1, CDRL2, and CDRL3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1860; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1919; and CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1978.

In specific examples, CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1683.

In specific examples, CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1742.

In specific examples, CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1801.

In certain examples, the antibody comprises a heavy chain complementarity determining region CDRH1, CDRH2, and CDRH3, wherein: CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1683; CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1742; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1801.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1861; CDRL2 is SEQ ID NO: 1920; CRDL3 is SEQ ID NO: 1979; CRDH1 is SEQ ID NO: 1684; CRDH2 is SEQ ID NO: 1743; and CDRH3 is SEQ ID NO: 1802.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1861; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1920; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1979; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1684; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1743; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1802.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1862; CDRL2 is SEQ ID NO: 1921; CRDL3 is SEQ ID NO: 1980; CRDH1 is SEQ ID NO: 1685; CRDH2 is SEQ ID NO: 1744; and CDRH3 is SEQ ID NO: 1803.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1862; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1921; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1980; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1685; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1744; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1803.

In specific examples, VH comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 210.

In specific examples, VL comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1036.

In specific examples, CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1862.

In specific examples, CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1921.

In specific examples, CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1980.

In certain examples, the antibody comprises a light chain complementarity determining region CDRL1, CDRL2, and CDRL3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1862; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1921; and CDRL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1980.

In specific examples, CDRH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1685.

In specific examples, CDRH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1744.

In specific examples, CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO. 1803.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1864; CDRL2 is SEQ ID NO: 1923; CRDL3 is SEQ ID NO: 1982; CRDH1 is SEQ ID NO: 1687; CRDH2 is SEQ ID NO: 1746; and CDRH3 is SEQ ID NO: 1805.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1864; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1923; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1982; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1687; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1746; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1805.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1865; CDRL2 is SEQ ID NO: 1924; CRDL3 is SEQ ID NO: 1983; CRDH1 is SEQ ID NO: 1688; CRDH2 is SEQ ID NO: 1747; and CDRH3 is SEQ ID NO: 1806.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1865; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1924; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1983; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1688; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1747; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1806.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1866; CDRL2 is SEQ ID NO: 1925; CRDL3 is SEQ ID NO: 1984; CRDH1 is SEQ ID NO: 1689; CRDH2 is SEQ ID NO: 1748; and CDRH3 is SEQ ID NO: 1807.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1866; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1925; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1984; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1689; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1748; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1807.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1872; CDRL2 is SEQ ID NO: 1931; CRDL3 is SEQ ID NO: 1990; CRDH1 is SEQ ID NO: 1695; CRDH2 is SEQ ID NO: 1754; and CDRH3 is SEQ ID NO: 1813.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1872; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1931; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1990; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1695; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1754; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1813.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1874; CDRL2 is SEQ ID NO: 1933; CRDL3 is SEQ ID NO: 1992; CRDH1 is SEQ ID NO: 1697; CRDH2 is SEQ ID NO: 1756; and CDRH3 is SEQ ID NO: 1815.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1874; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1933; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1992; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1697; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1756; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1815.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1875; CDRL2 is SEQ ID NO: 1934; CRDL3 is SEQ ID NO: 1993; CRDH1 is SEQ ID NO: 1698; CRDH2 is SEQ ID NO: 1757; and CDRH3 is SEQ ID NO: 1816.

In specific examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1875; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1934; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1993; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1698; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1757; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1816.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1878; CDRL2 is SEQ ID NO: 1937; CRDL3 is SEQ ID NO: 1996; CRDH1 is SEQ ID NO: 1701; CRDH2 is SEQ ID NO: 1760; and CDRH3 is SEQ ID NO: 1819.

In some examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1878; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1937; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1996; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1701; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1760; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1819.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1882; CDRL2 is SEQ ID NO: 1941; CRDL3 is SEQ ID NO: 2000; CRDH1 is SEQ ID NO: 1705; CRDH2 is SEQ ID NO: 1764; and CDRH3 is SEQ ID NO: 1823.

In further examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1882; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1941; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 2000; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1705; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1764; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1823.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 is SEQ ID NO: 1886; CDRL2 is SEQ ID NO: 1945; CRDL3 is SEQ ID NO: 2004; CRDH1 is SEQ ID NO: 1709; CRDH2 is SEQ ID NO: 1768; and CDRH3 is SEQ ID NO: 1827.

In certain examples, the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein: CDRL1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1886; CDRL2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1945; CRDL3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 2004; CRDH1 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1709; CRDH2 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1768; and CDRH3 comprises a sequence at least 60% (for example, at least 60%, at least 65%, at least 70%, at least 75%, at least 80%, at least 85%, at least 90%, at least 95%, at least 96%, at least 97%, at least 98%, at least 99%) identical to SEQ ID NO: 1827.

In some examples, the recombinant antibody binds to at least one coronavirus antigen. In some embodiments, the recombinant antibody binds to at least one SARS-CoV-2 antigen.

In some examples, the coronavirus is SARS-CoV-2.

Also provided herein is a method for detecting a coronavirus infection in a subject, comprising: providing a biological sample from the subject, and detecting a coronavirus antigen in the biological sample with an antibody that specifically binds to the coronavirus antigen, wherein the antibody is disclosed herein.

The biological sample can be from, for example, a throat swab, a nasal swab, a nasopharyngeal swab, an oropharyngeal swab, cells, blood, serum, plasma, saliva, urine, stool, sputum, or nasopharyngeal aspirates.

In some embodiments, the coronavirus infection is caused by SARS-CoV-2. In some embodiments, the method comprises contacting the biological sample with a SARS-CoV-2 antigen. In some embodiments, the SARS-CoV-2 antigen is directly immobilized on a substrate and is detected by an antibody disclosed herein directly or indirectly by a labeled heterologous anti-isotype antibody, wherein the bound antibody can be detected by a detection assay. The SARS-CoV-2 antigen can be selected from the spike (S), envelope (E), membrane (M), and nucleocapsid (N) proteins, or a fragment thereof.

The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a secondary antibody that is labeled a fluorescent probe or with biotin for detection. In vitro techniques for detection of the antibodies of SARS-CoV-2 include enzyme linked immunosorbent assays (ELISAs), Western blots, immunoprecipitations and immunofluorescence, IgM antibody capture enzyme immunoassay (MAC-ELISA), indirect IgG ELISA, indirect fluorescent antibody assay (IFAT), hemagglutination inhibition (HIT), and serum dilution cross-species plaque reduction neutralization tests (PRNTs).

In some embodiments, in vitro techniques for detection of an antigen of SARS-CoV-2 include enzyme linked immunosorbent assays (ELISAs), Western blots, immunoprecipitations and immunofluorescence. Furthermore, in vivo techniques for detection of SARS-CoV-2 include introducing into a subject a labeled antibody directed against the polypeptide. For example, the antibody can be labeled with a radioactive marker whose presence and location can be detected by standard imaging techniques, including autoradiography.

In some embodiments, the levels of the antibodies are determined by immunoassay comprising Enzyme linked immunospot (ELISPOT), Enzyme-linked immunosorbent assay (ELISA), western blot, or a multiplex ELISA assay. In some embodiments, the multiplex ELISA assay is selected from the group consisting of Luminex, Veriplex, LEGENDplex, Bio-Plex, Milliplex MAP, and FirePlex. The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

The invention also encompasses kits for detecting the presence of SARS-CoV-2 or a polypeptide/antigen thereof in a biological sample. For antibody-based kits, the kit can comprise, for example: (1) a first antibody (e.g., attached to a solid support) which binds to a coronavirus antigen; and, optionally, (2) a second, different antibody which binds to either the coronavirus antigen or the first antibody and is conjugated to a detectable agent.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation will be required to optimize such process conditions.

### Example 1: SARS-CoV-2 Antibodies from Children Exhibit Broad Neutralization and Belong to Adult Public Clonotypes

Motivated by the established significance of SARS-CoV-2-neutralizing antibodies in adults, characterized herein are SARS-CoV-2-specific antibody repertoires in a young cohort of individuals aged from 5 months to 18 years old. Results show that neutralizing antibodies in children possess similar genetic features compared to antibodies identified in adults, with multiple antibodies from children belonging to previously established public antibody clonotypes in adults. Notably, antibodies from children showed potent neutralization of circulating SARS-CoV-2 variants that have cumulatively resulted in resistance to virtually all approved monoclonal antibody therapeutics. Results show that children can rely on similar SARS-CoV-2 antibody neutralization mechanisms compared to adults and are an underutilized source for the discovery of effective antibody therapeutics to counteract the ever-evolving pandemic.

The human immune response to a pathogen depends on a wide variety of factors, including the history of prior exposure to the same or related pathogens, as well as the current state of an individual's immune system. Compared to adults, children's infection and vaccination histories are more limited and their immune systems are not fully mature. As a result, substantial differences in disease phenotype can be observed for a variety of infectious diseases between children and adults. (Ruf, 2014; Shafagati, 2018; Shi, 2017.) In particular, children tend to be more susceptible to severe disease phenotypes for several common infectious diseases, including influenza, respiratory syncytial virus, human metapneumovirus, and others. (Ruf, 2014; Shafagati, 2018; Shi, 2017.) However, this trend appeared to be reversed in the case of SARS-CoV-2, with children experiencing significantly less severe disease phenotypes compared to adults. (Zimmermann, 2021; Mehta, 2020.) Adolescents also have less incidence of severe COVID-19, and those with severe disease exhibit lower rates of hospitalization, require shorter hospital stays, and have lower mortality than adults. (Chou, 2022.) While there have been some efforts aiming to understand these differences in how children and adults respond to SARS-CoV-2 infection, little is still known about the specific factors that are responsible for this phenomenon.

While a number of immune system components have been implicated in the response to SARS-CoV-2 infection and vaccination (Mathew, 2020; Kramer, 2022; Diamond, 2022), antibodies that are capable of effectively neutralizing the virus are among the most critical factors (Cromer, 2022). In fact, the elicitation of neutralizing antibodies to the spike glycoprotein of SARS-CoV-2 is the primary target for the majority of current vaccines on the market. (Krammer, 2020; Wang, 2021) The initial SARS-CoV-2 vaccine candidates were developed based on the knowledge that neutralizing antibodies previously had been shown to protect against infection with a variety of other pathogens (Corbett, 2020), long before ultimately showing that such antibodies are a correlate of protection against SARS-CoV-2 as well (Cromer, 2022; Khoury, 2021). Antibody responses to SARS-CoV-2 infection and vaccination have been extensively studied in adults, resulting in a plethora of information about the genetic, molecular, and functional characteristics of SARS-CoV-2-specific antibodies. (Kramer, 2022; Wang, 2021; Collier, 2021; Cele, 2022.) While antibodies in adults have been found to target a variety of different epitopes on the spike glycoprotein, several epitope clusters have been identified that are commonly targeted at the population level (Barnes, 2020). The ability of different individuals to target similar epitopes is in part attributed to the existence of “public” antibodies with highly similar genetic features that are found in many individuals (Setliff, 2018; Chen, 2021). In practice, public antibodies in the context of SARS-CoV-2 have been defined based on the use of identical combinations of variable heavy (VH) and light genes (VL) (Kramer, 2022; Tan, 2021), although more stringent requirements related to high sequence identity in the third complementarity determining regions of the heavy and light chains (CDRH3 and CDRL3, respectively) can also be incorporated (Setliff, 2018; Chen, 2021). Overall, the identification of a variety of public antibodies indicates that the immune system in adults can converge on a few common modes of SARS-CoV-2 spike recognition that can effectively counteract the virus.

Within the first few months of the pandemic, monoclonal antibodies (mAbs) isolated from adults were translated into the clinic as both preventive and therapeutic measures (Zost, 2020; Liu, 2020; Pinto, 2020; Rogers, 2020; Shi, 2020; Jones, 2020; Hansen, 2020; Sun, 2020). The critical significance of translating multiple mAbs with different genetic, epitope, and functional characteristics into the clinic became especially evident once the effects of SARS-CoV-2 variant evolution came to the forefront. While the approved mAbs were initially effective against the wildtype virus, mutations within the spike protein soon enabled viral escape from some, and now virtually all, mAbs on the market. (Weisblum, 2020; Chen, 2021; Wang, 2021; Wang, 2022; Wang, 2023; Sheward, 2022; Cao, 2022; Imai, 2023.) Further efforts toward the development of next-generation mAbs with an expanded breadth of SARS-CoV-2 variant reactivity is currently ongoing. (Shiakolas, 2022; Cao, 2022; Astrazeneca, 2022.)

Unlike the expansive studies of antibody responses to SARS-CoV-2 in adults, there is little to no information about the types and specificities of neutralizing antibodies in children, with studies generally limited to the assessment of polyclonal responses. (Dowell, 2022; Fraley, 2021; Yang, 2021) To address this, LIBRA-seq was employed, a powerful tool that combines a DNA oligo barcoded antigen library, fluorescence-activated cell sorting (FACS), next-generation sequencing (NGS), and a computational pipeline to enable high-throughput mapping of antibody sequence to antigen specificity, while maintaining single-cell resolution with high precision. (Setliff, 2019) PBMCs from children are generally challenging to acquire, and LIBRA-seq facilitated the utilization of limited samples to efficiently discover cross-reactive B cells predicted to bind the spike protein of SARS-CoV-2, its variants, and other betacoronaviruses. Due to the different disease phenotypes observed in children compared to adults and the established significance of the antibody response to SARS-CoV-2 in adults, characteristics of SARS-CoV-2-specific antibodies in children were explored. In this work, similarities and differences between SARS-CoV-2 antibodies in children were delineated and previously characterized antibody responses in adults, focusing on both genetic features and functional characteristics like epitope specificity and neutralization phenotypes. The results presented here highlight the properties of SARS-CoV-2-specific antibody responses in children, emphasize the potential of samples from children as an underutilized source for novel mAb candidates, and can help inform the design of next-generation pediatric vaccines.

### Results

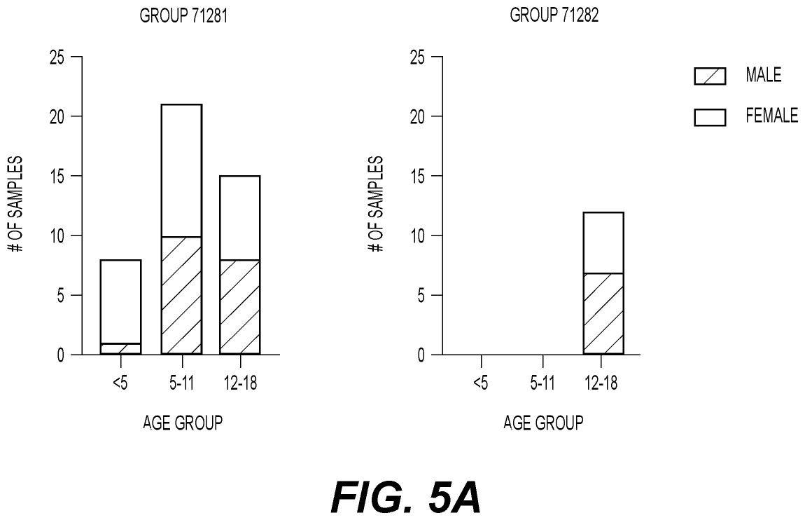

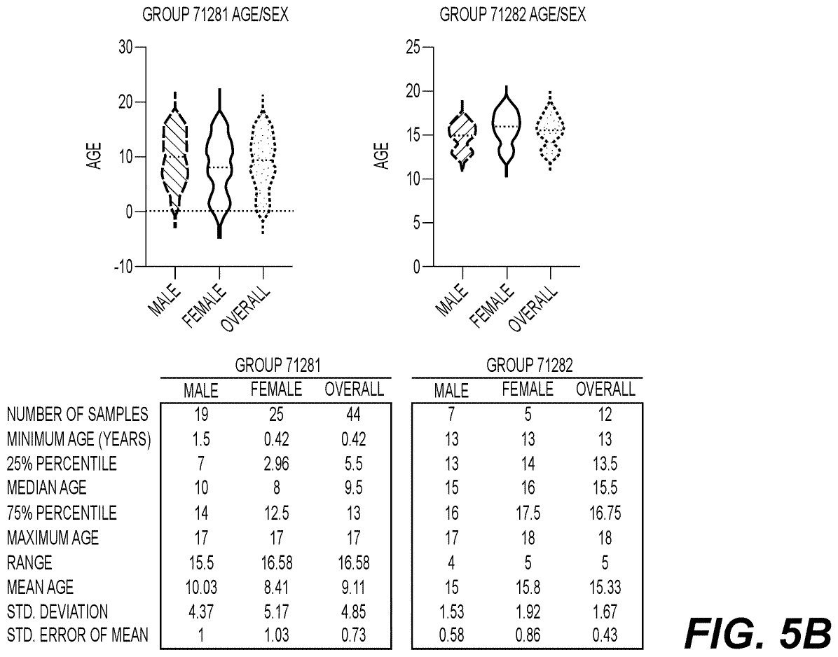

Cohort of samples from children. Peripheral blood mononuclear cells (PBMCs) were obtained from peripheral blood samples remaining after diagnostic tests were performed in the Clinical Hematology Laboratory at Vanderbilt University Medical Center (VUMC). Samples were collected for children up to 18 years of age between July-August 2021. Samples were divided into two groups: 71281, No known exposure to SARS-CoV-2 infection or vaccination; and 71282, Known exposure to SARS-CoV-2 infection or vaccination, determined through self-reporting. Group 1 included a total of 44 samples, ages ranging from 5 months-17 years, and a median age of 9.5, while group 2 included a total of 12 samples, ages ranging from 13-18 years, and a median age of 15.5 (FIGS. 5A-5B). Due to low sample volumes (ranging from 200 μL to 3 mL, average ~1 mL), all samples within a group were pooled together for subsequent characterization.

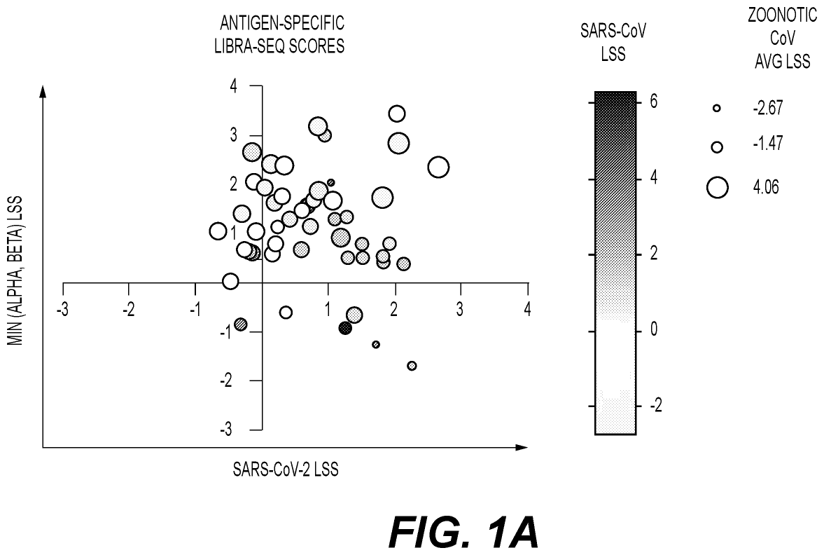

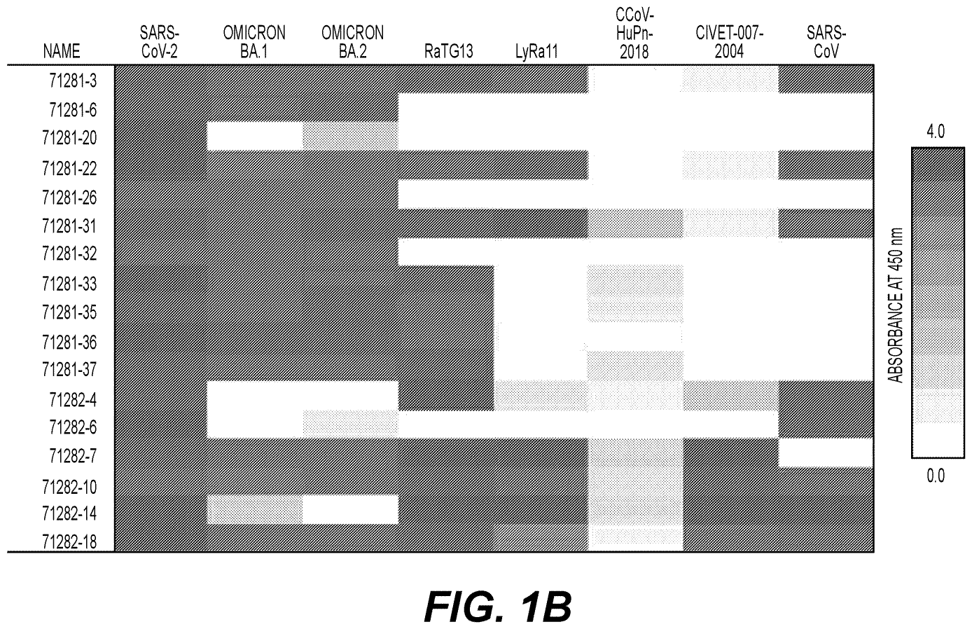

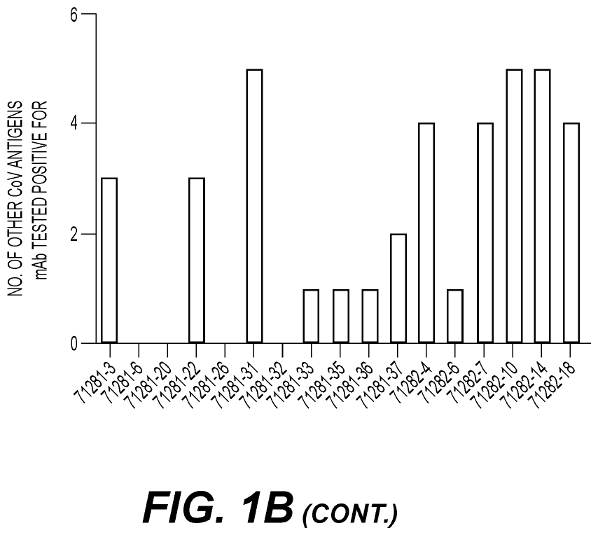

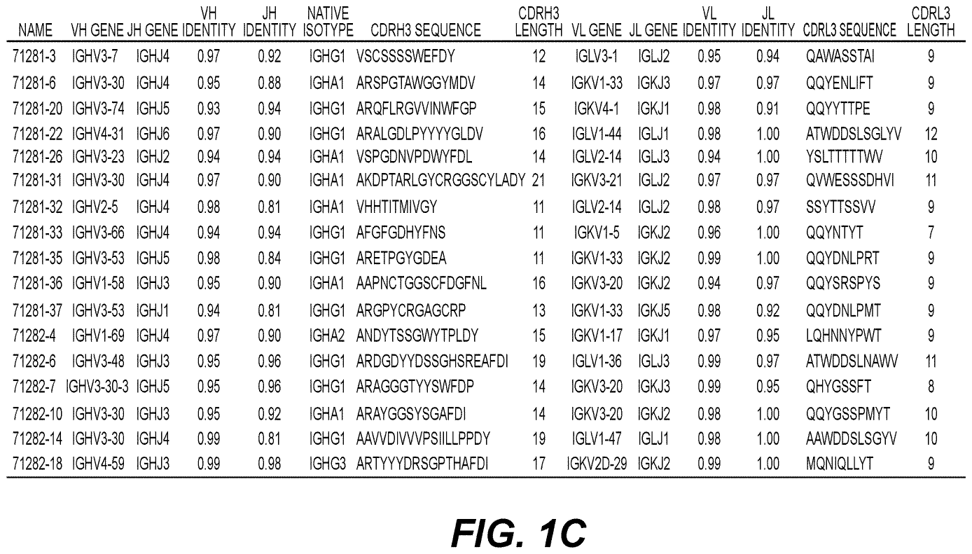

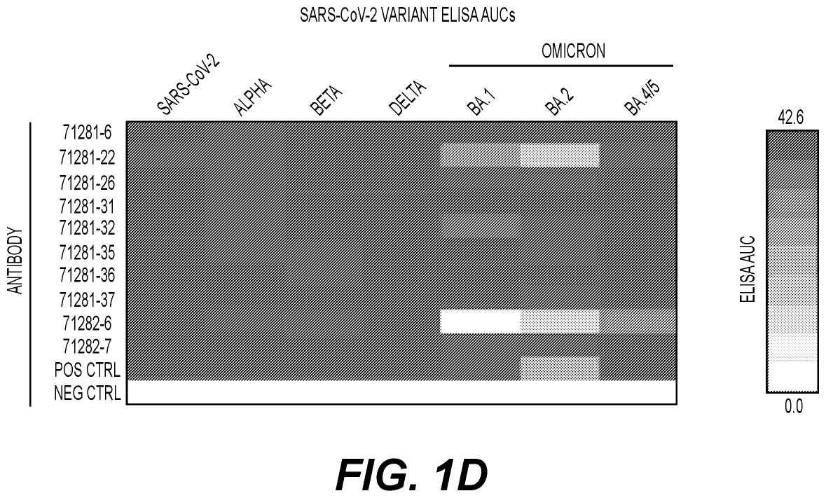

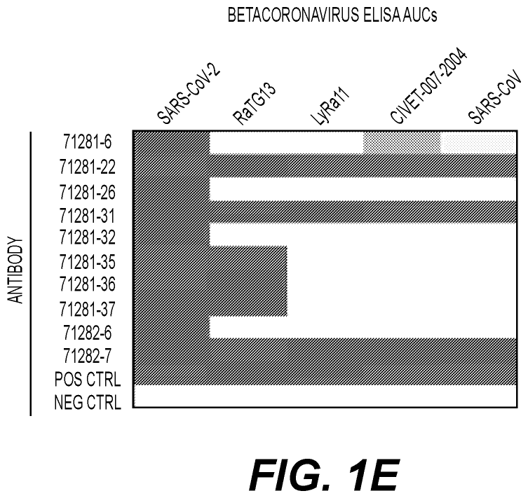

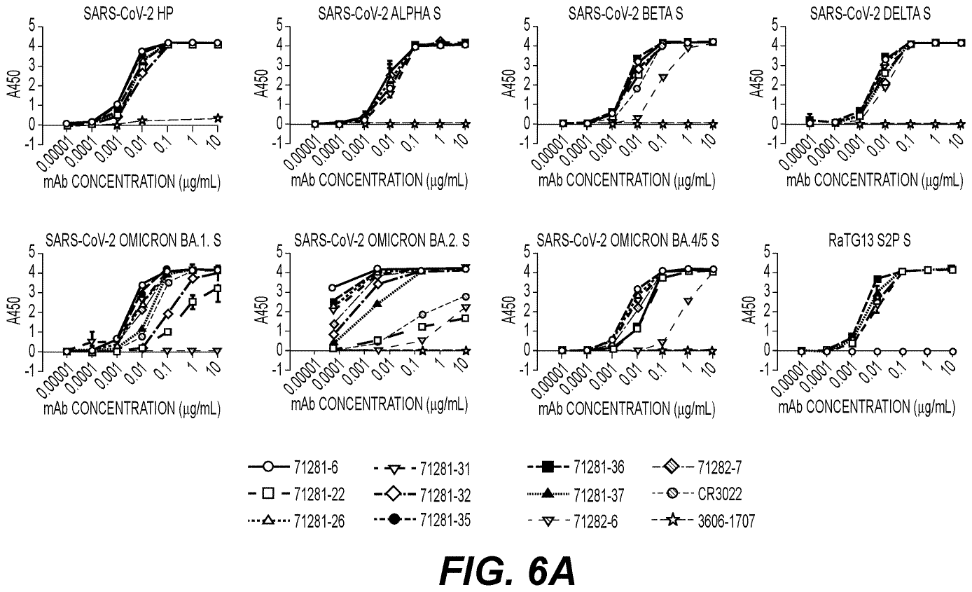

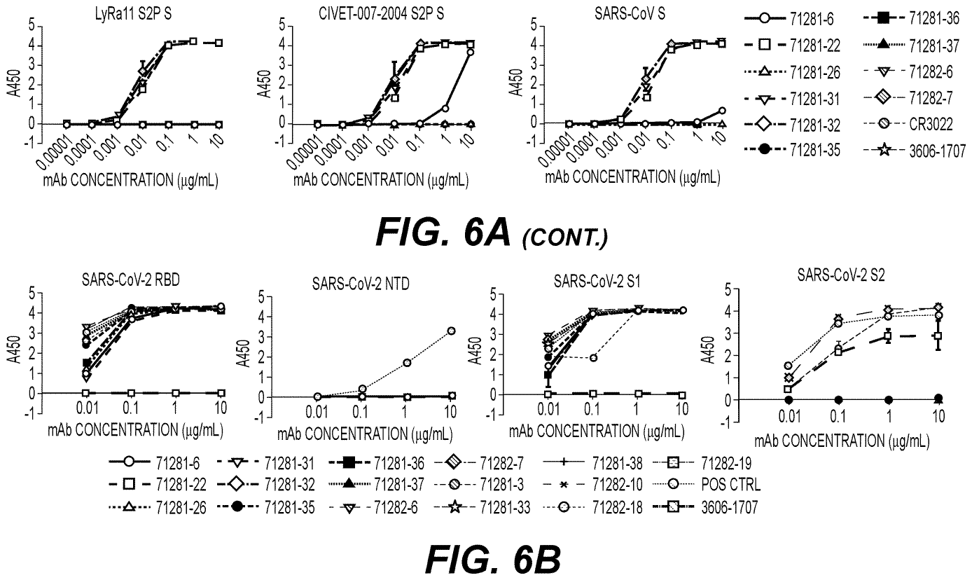

Discovery of SARS-CoV-2 antibodies from children's samples. For each of the two sample groups, a LIBRA-seq discovery experiment was performed to determine the cross-reactivity patterns of SARS-CoV-2+B cells from children. The antigen screening library incorporated a panel of stabilized recombinant spike antigens from SARS-CoV-2 variants (WA1, alpha, and beta), and from other sarbecoviruses with potential for zoonosis (SARS-CoV, RaTG13, LYRa11, and Civet-007-2004). Applying this antigen library to the two groups of samples described above resulted in the identification of a number of B cells with predicted cross-reactivity between multiple SARS-CoV-2 variants, multiple sarbecoviruses, or both (FIG. 1A). Based on these LIBRA-seq results, a set of B cells was prioritized for further characterization as recombinant IgG1 monoclonal antibodies. Microscale expression of paired heavy-light chains (see Methods), followed by antigenicity characterization by ELISA, revealed 17 antibodies with breadth within SARS-CoV-2 and/or other non-SARS-CoV-2 coronavirus recognition, from each of the two groups of samples (FIG. 1B). These antibodies derived from multiple different isotypes (IgG1, IgG3, and IgAQ1), and utilized diverse heavy and light chain variable genes, with varying CDRH3 and CDRL3 lengths (CDRH3: 11-21 amino acids; CDRL3: 7-12 amino acids) and somatic hypermutation levels (heavy chain: 1-7%; light chain: 1-6%) (FIG. 1C). Antibody leads were then expressed in large-scale, purified, and tested for recognition of an expanded set of antigens. The antibodies showed diverse antigen specificities, with recognition of different subsets of SARS-CoV-2 variants, including the omicron lineage variants BA.1, BA.2, and BA.4/5, (FIGS. 1D and 6A-6B) or other sarbecoviruses (FIGS. 1E and 6A-6B). Together, these results revealed the existence of SARS-CoV-2 antibodies in samples from children with a wide range of breadth within SARS-CoV-2 and related sarbecoviruses.

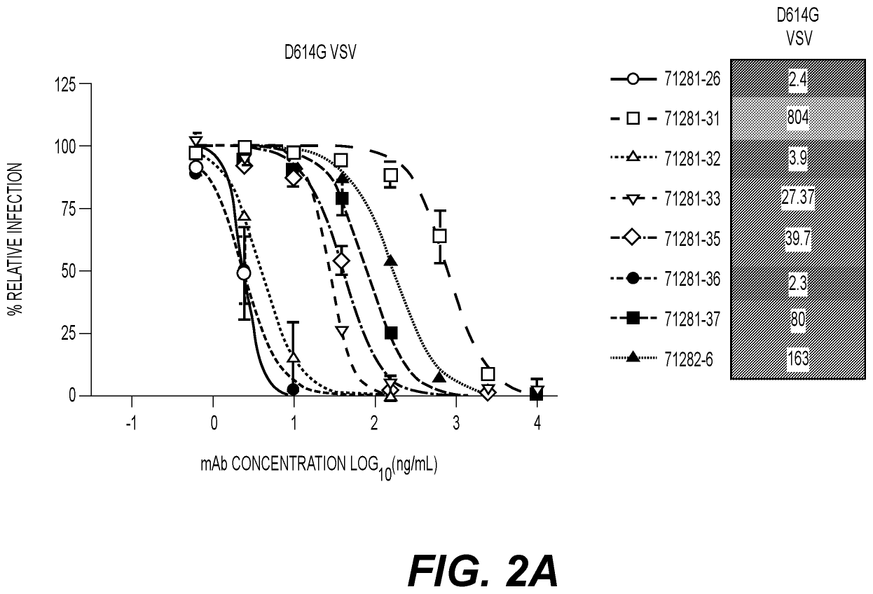

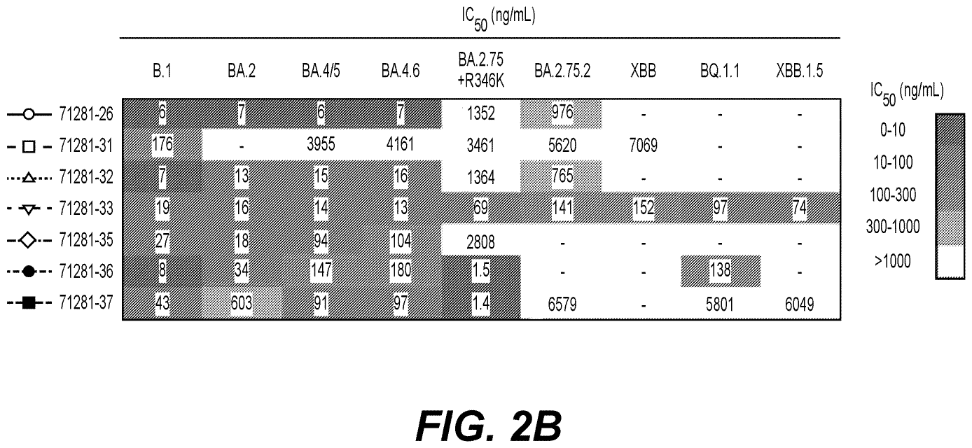

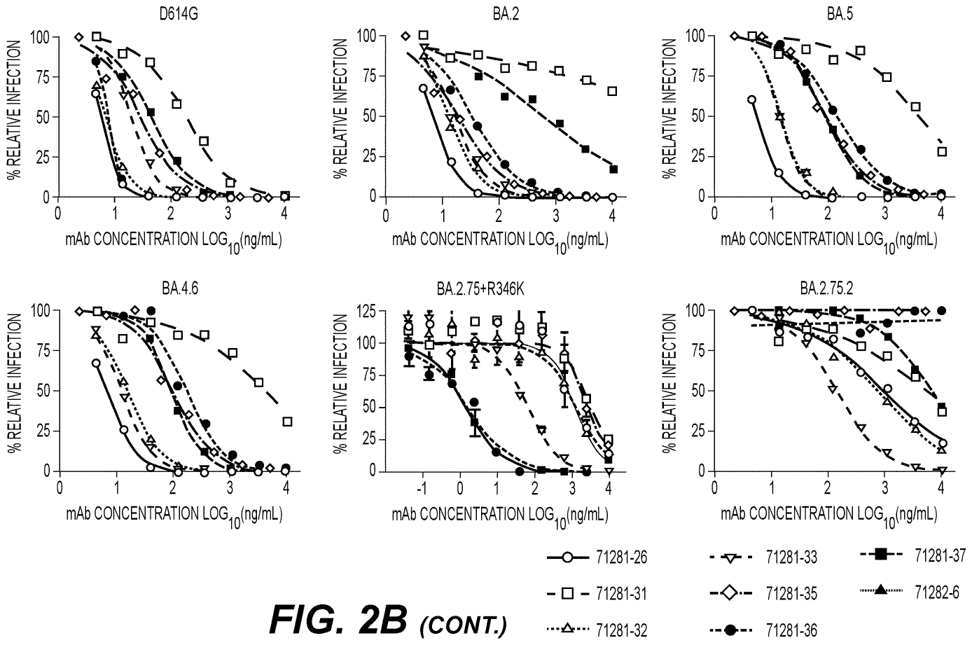

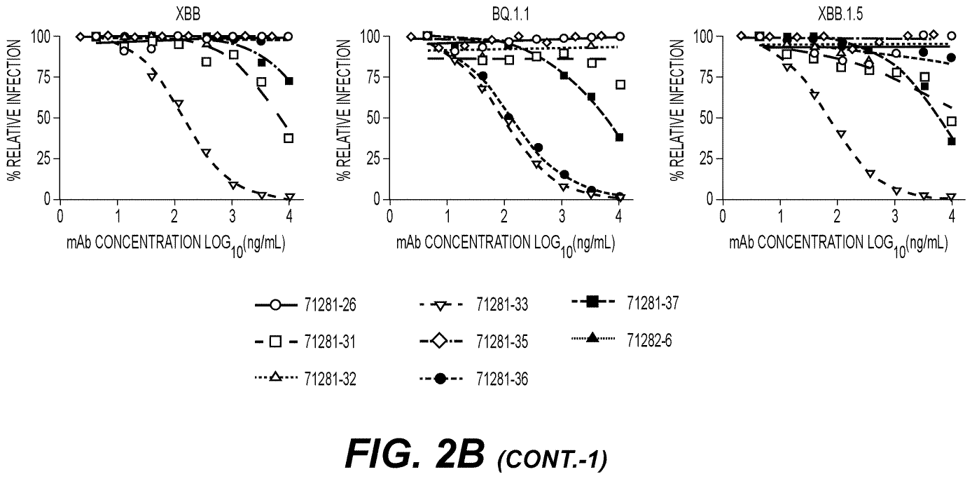

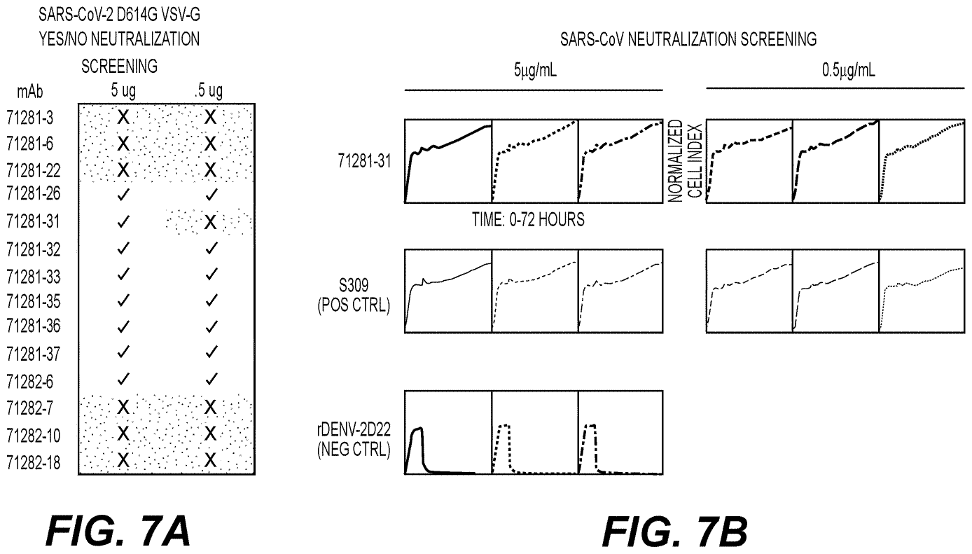

Antibodies from children neutralize recently emergent SARS-CoV-2 variants. It was determined whether the SARS-CoV-2 antibodies identified from children's samples exhibit neutralization breadth and potency against diverse SARS-CoV-2 variants (FIG. 2). Indeed, 8 of the 14 antibodies from children that showed binding to at least one SARS-CoV-2 variant or SARS-CoV, when tested in a vesicular stomatitis virus (VSV) based neutralization assay (Case, 2020; Suryadevara, 2022), potently neutralized the SARS-CoV-2 variant harboring the D614G mutation (Zhang, 2020), with a half maximal inhibitory concentration (IC₅₀) of <10 ng/mL observed for 3 antibodies (FIGS. 2A and 7A). Similar neutralization patterns were also observed against the D614G variant in a pseudotyped virus-based neutralization assay (FIG. 2B). The pseudotyped virus assay was also used for evaluating the breadth of the antibodies against recently emergent omicron variants that have been associated with resistance to virtually all available monoclonal antibody therapies. (Wang, 2023; Sheward, 2022; Imai, 2023; Cao, 2022) Notably, 6 of the 7 antibodies from children exhibited neutralization breadth against 4 or more of the tested variants with IC₅₀ <1 μg/mL, and all of the tested antibodies neutralized BA.4/5 (FIG. 2B). In particular, antibody 71281-33 retained neutralization potency against all 9 SARS-CoV-2 pseudovirus variants tested: D614G, BA.2, BA.2.75+R346T, BA.4, BA.4.6, BA.2.75.2, XBB, BQ.1.1, and XBB.1.5, the last 5 of which have been implicated with resistance to monoclonal antibody neutralization, including to bebtelovimab, Evusheld (a cocktail of tixagevimab and cilgavimab), and sotrovimab. (Wang, 2023; Sheward, 2022; Imai, 2023; Cao, 2022) Together, these data show that antibodies identified from children's samples can display high levels of neutralization breadth and potency against SARS-CoV-2 variants even without prior vaccination or exposure to that variant. More generally, these results indicate that children's samples can play an important role in the discovery of effective SARS-CoV-2 antibody therapeutics.

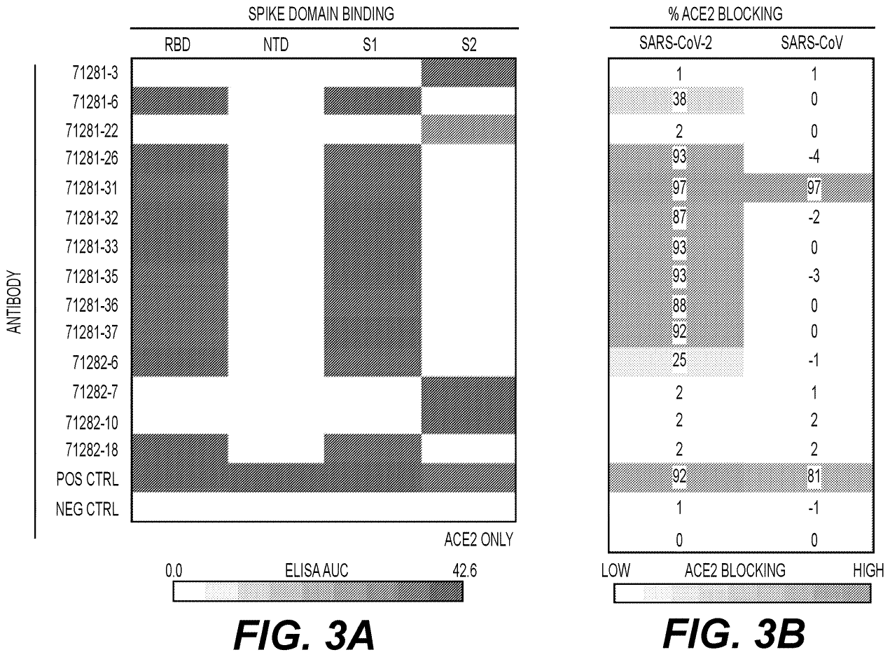

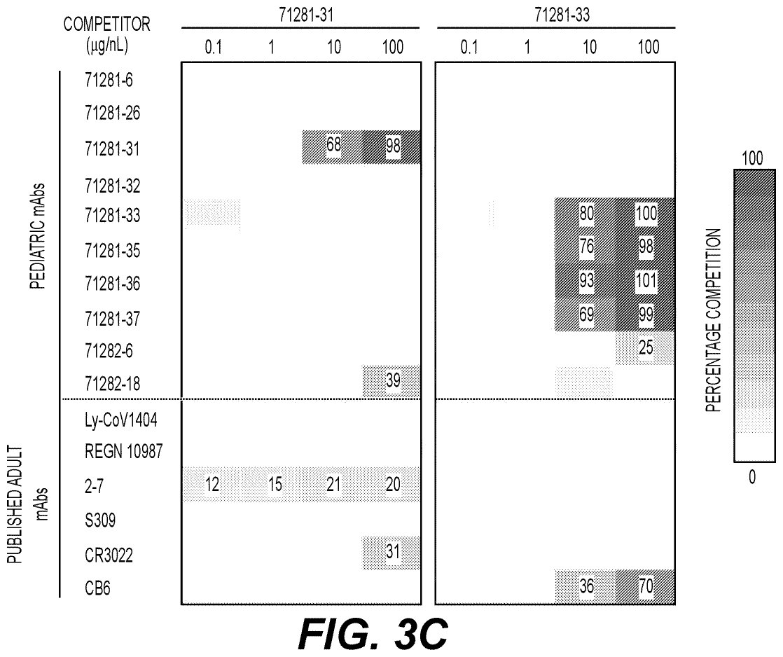

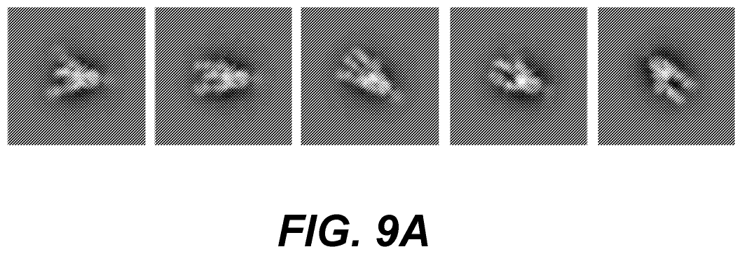

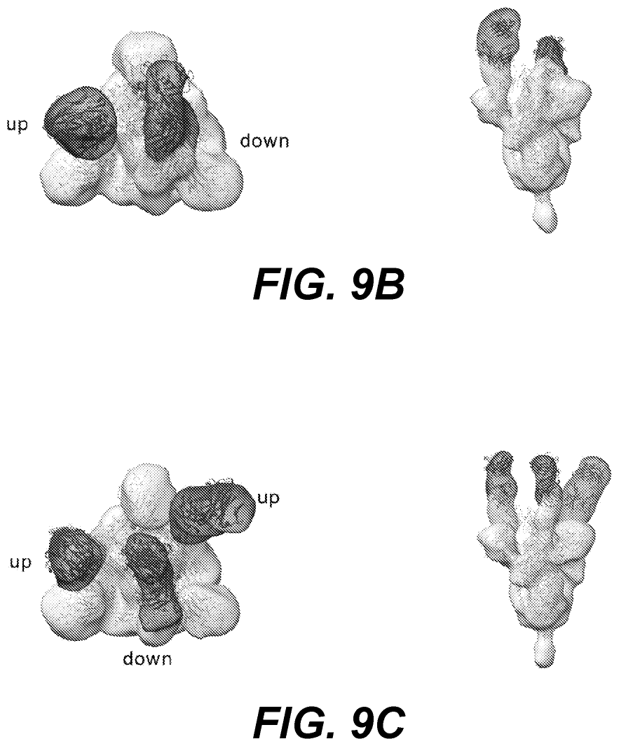

Antibodies from children target multiple diverse epitopes on the SARS-CoV-2 spike. To evaluate the breadth of epitopes targeted by antibodies from children, binding against different spike subdomains (FIGS. 3A and 6B) was tested. Both receptor-binding domain (RBD) and S2 subdomain antibodies were identified. Further, antibody competition for spike-receptor ACE2 binding was tested in an ELISA assay, revealing high levels (>85%) of ACE2 blocking activity by 7/10 RBD-specific antibodies and medium levels (25-38%) of blocking activity by 2 of the remaining RBD-specific antibodies (FIG. 3B). Notably, antibody 71281-31 showed strong ACE2 blocking ability against both SARS-CoV-2 and SARS-CoV, in line with the SARS-CoV/SARS-CoV-2 cross-reactivity (FIG. 1E) and RBD recognition (FIG. 3A) observed for this antibody. Antibody 71281-31 was also able to neutralize both SARS-CoV-2 (FIG. 2A) and SARS-CoV (FIG. 7B), albeit at low potency, indicating a potential tradeoff between high breadth of virus recognition and neutralization potency. As a next step, the exceptionally broadly SARS-CoV-2 neutralizing antibody 71281-33 and the SARS-CoV/SARS-CoV-2 cross-reactive antibody 71281-31 were selected to further delineate their epitope regions in antibody competition assays, both against other antibodies from children, as well as against published adult SARS-CoV-2 antibodies (FIG. 3C). Antibody 71281-31 did not compete with any other antibodies, including the published S309 RBD SARS-CoV/SARS-CoV-2 reactive antibody (Pinto, 2020), suggesting it targets a unique cross-reactive epitope. In contrast, antibody 71281-33 competed with a subset of the other antibodies from children, and to some extent with the published class 1 RBD antibody CB6 (etesevimab) (Shi, 2020), but no other antibodies in the set that was tested. Next, negative-stain electron microscopy experiments (nsEM) were performed in order to further delineate the epitope of neutralizing antibody 71281-33. Reconstructive 3D mapping of the SARS-CoV-2 spike in complex with Fab 71281-33 indicated that the epitope of that antibody is near the receptor-binding motif (RBM) of the RBD. Notably, the nsEM reconstruction showed Fab 71281-33 binding to the RBD in both the up and down states (FIG. 9). This epitope, along with the IGHV3-66 gene usage, short CDRH3, and mild competition with CB6, indicate that mAb 71281-33 is likely a class 1 RBD antibody. (Barnes, 2020) Together, these results suggest that the SARS-CoV-2 antibodies were identified from children samples target a variety of different epitopes both on the spike in general, and within the RBD subdomain.

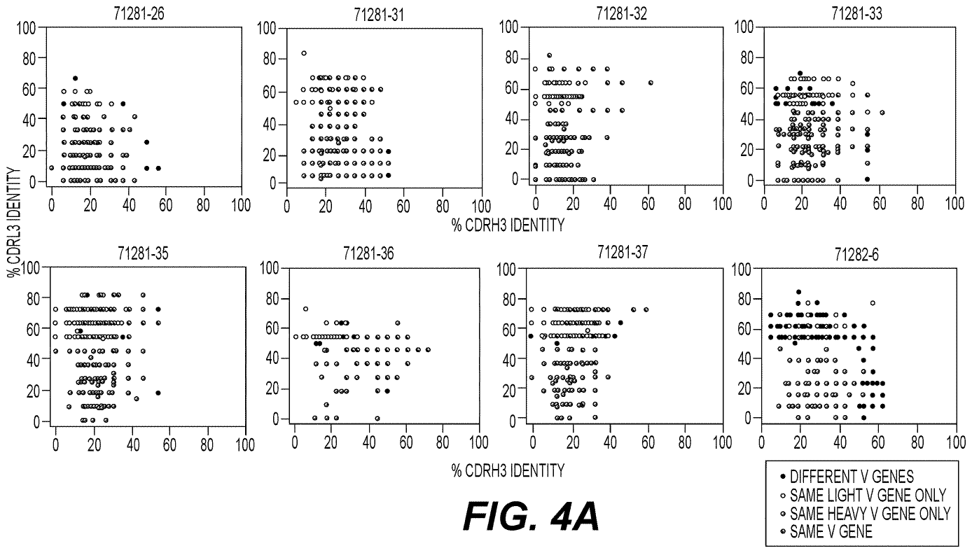

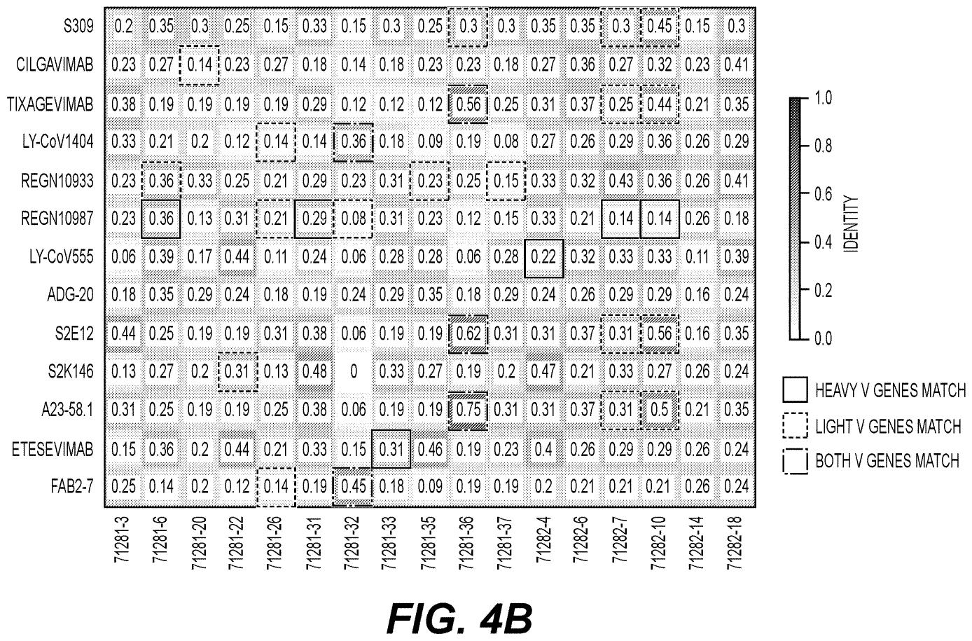

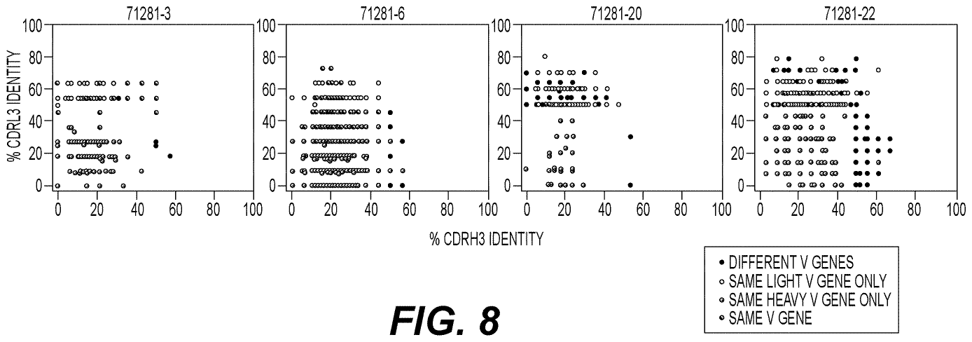

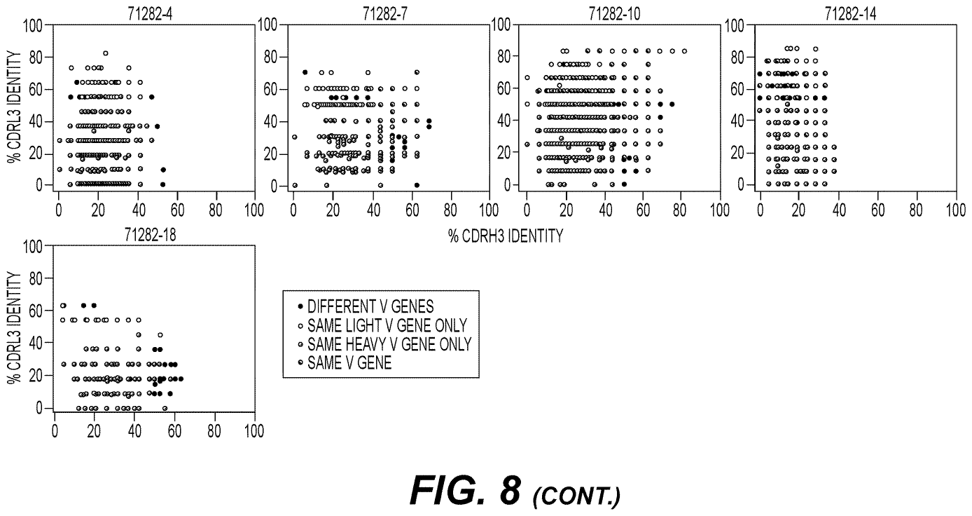

Antibodies from children have sequence features that are associated with known public clonotypes in adults. Characterization of the antibody responses to SARS-CoV-2 infection and vaccination in adults has led to the discovery of common genetic characteristics for SARS-CoV-2-reactive antibodies, that are observed in multiple individuals. (Barnes, 2020; Chen, 2021; Tan, 2021; Kim, 2021; Cao, 2020) Such antibodies define public clonotypes and highlight a convergent mechanism of viral antigen recognition that can be observed at the population level. (Barnes, 2020; Setliff, 2018; Chen, 2021; Tan, 2021; Yang, 2021.) Antibodies discovered here were compared to known SARS-CoV-2-reactive antibodies in adults. To that end, utilizing the genetic features identified in FIG. 1C, SARS-CoV-2-reactive antibodies identified here were compared to a database of published SARS-CoV-2 antibody sequences. (Raybould, 2021) Notably, 16 of 17 antibodies that showed binding to SARS-CoV-2 spike utilized a combination of variable heavy and light chain genes that was identical to previously published adult SARS-CoV-2 antibodies (FIGS. 4A and 8). Of these, 11 antibodies also had CDRH3 identity of >50% to at least one published adult SARS-CoV-2 antibody, and 4 antibodies had CDRH3 identity of >70% (FIGS. 4A and 8). For all of the spike-binding antibodies from children, previously published SARS-CoV-2 antibodies were identified that utilized an identical variable heavy chain gene, independent of the variable light gene, with 15 of the antibodies having CDRH3 identity of >50% to at least one published adult SARS-CoV-2 antibody, and 5 antibodies having CDRH3 identity of >70% (FIGS. 4A and 8). Specifically focusing on the subset of 8 antibodies from children that were found to be neutralizing, all 8 utilized a combination of variable heavy and light genes that was identical to previously published adult SARS-CoV-2 antibodies, and 5 of the 8 also had CDRH3 identity of >50% to at least one published adult SARS-CoV-2 antibody (FIG. 4A). Some of these combinations included well-documented public SARS-CoV-2 clonotypes that have been defined as public clonotypes specifically based on the variable gene usage, including IGHV1-58/IGKV3-20 (Dong, 2021; Tortorici, 2020; Wang, 2022; Chen, 2021) (used by antibody 71281-36) and IGHV2-5/IGLV2-14 (Westendorf, 2022; Yuan, 2022; Liu, 2020) (utilized by 71281-32), as well as the IGHV3-53/3-66 public class of SARS-CoV-2 antibodies (Wang, 2022; Chen, 2021; Zhang, 2021) (utilized by 71281-33, 71281-35, and 71281-37).

In addition to the large-scale database comparison, the similarity of the antibodies identified here were evaluated to a set of marketed, in development, or well-characterized SARS-CoV-2 antibodies. Of note, several of the antibodies from children belonged to the same clonotypes as these published monoclonal antibodies. In particular, 71281-32 shared identical variable heavy and light chain gene usage with LY-CoV1404 (bebtelovimab) and 2-7, and 71281-36 shared identical variable heavy and light chain genes with tixagevimab, S2E12, and A23-58.1, with CDRH3 identities of 56%, 62%, and 75%, respectively (FIG. 4B). Together, these results suggest that the development of SARS-CoV-2 antigen specificity may use similar antibody sequence solutions in children as in adults.

### Discussion

The state-of-the-art technologies that have revolutionized the field of antibody research in recent years have had limited applications in the context of children, likely due to the difficulty of obtaining and studying samples from children. Given the established importance of antibodies as a major component of immune responses to infection and vaccination, efforts aiming to fill this gap in knowledge can have significant implications for basic immunology, through shedding light onto the fundamental rules that govern antigen recognition by antibodies in children. Such efforts can also be of clinical and translational significance, by providing insights for the development of novel mAb therapies and vaccine modalities that are specifically tailored toward children.

Due to the significant differences in disease phenotype between children and adults, studying antigen-specific antibody repertoires in children in the context of SARS-CoV-2 is well-motivated. To that end, using the LIBRA-seq technology for high-throughput antibody discovery, a number of SARS-CoV-2 spike-specific antibodies were identified and characterized from a cohort of children. These antibodies targeted a variety of different spike epitopes and were associated with diverse genetic features, suggesting a broad portfolio of potential solutions for antigen targeting in children. Moreover, several of the antibodies from children belonged to known classes of public antibodies from adults, suggesting that there is a convergence of antigen-specific antibody responses for a wide range of ages. While child and adult antibodies can belong to overlapping public clonotypes based on common variable gene usage, there can be sufficient differences in the antibody sequence (e.g., in the CDRH3 region, FIG. 4A) that could lead to major differences in the neutralization sensitivity phenotypes for these antibodies (FIG. 2B) compared to adult antibodies from the same public clonotypes. This indicates that optimization of the antibody neutralization phenotype can be achieved through fine-tuning of antigen recognition even within individual public clonotypes. (Dong, 2021; Starr, 2021; Kramer, 2021; Tan, 2021.)

Notably, a set of antibodies from children showed potent neutralization of different currently circulating SARS-CoV-2 variants, including variants that have successfully escaped from the currently available mAb products. This result points to samples from children as a potential source for the development of new mAb candidates with improved breadth of SARS-CoV-2 reactivity, and further highlights the importance of deciphering the antigen-specific antibody repertoires in children for other infectious diseases with high biomedical significance.

This study investigates the genetic features, antigen cross-reactivity, and coronavirus neutralization breadth of antibodies discovered in a cohort of children aged 5 months to 18 years old. Due to the limited number of samples that were obtained in the lower end of the age range (0-5 years), samples were pooled, and group 71282 consisted of samples from only adolescent ages 12-17. Studies that include stratified children's age groups with retained donor source information for the identified B cells can better reflect the different characteristics of B cell repertoires in children. Additionally, while samples from group 71281 had no history of infection or vaccination, this could not be experimentally confirmed, so the possibility that donors in that cohort had been exposed at some point could not be ruled out. Finally, public clonotype analysis was defined purely through sequence features. A comprehensive mapping of epitopes was performed to confirm that adult and child antibodies which meet the sequence criteria to be defined within the same clonotype, bind the spike protein in near-identical ways.

TABLE 1
Antibodies analyzed and discussed in Example 1.

| Name | HC | LC | CDRL1 | CDRL2 | CDRL3 | CDRH1 | CDRH2 | CDRH3 |
|---|---|---|---|---|---|---|---|---|
| 71281-3 | 180 | 1006 | 1832 | 1891 | 1950 | 1655 | 1714 | 1773 |
| 71281-6 | 183 | 1009 | 1835 | 1894 | 1953 | 1658 | 1717 | 1776 |
| 71281-20 | 197 | 1023 | 1849 | 1908 | 1967 | 1672 | 1731 | 1790 |
| 71281-22 | 199 | 1025 | 1851 | 1910 | 1969 | 1674 | 1733 | 1792 |
| 71281-26 | 203 | 1029 | 1855 | 1914 | 1973 | 1678 | 1737 | 1796 |
| 71281-31 | 208 | 1034 | 1860 | 1919 | 1978 | 1683 | 1742 | 1801 |
| 71281-32 | 209 | 1035 | 1861 | 1920 | 1979 | 1684 | 1743 | 1802 |
| 71281-33 | 210 | 1036 | 1862 | 1921 | 1980 | 1685 | 1744 | 1803 |
| 71281-35 | 212 | 1038 | 1864 | 1923 | 1982 | 1687 | 1746 | 1805 |
| 71281-36 | 213 | 1039 | 1865 | 1924 | 1983 | 1688 | 1747 | 1806 |
| 71281-37 | 214 | 1040 | 1866 | 1925 | 1984 | 1689 | 1748 | 1807 |
| 71282-4 | 220 | 1046 | 1872 | 1931 | 1990 | 1695 | 1754 | 1813 |
| 71282-6 | 222 | 1048 | 1874 | 1933 | 1992 | 1697 | 1756 | 1815 |
| 71282-7 | 223 | 1049 | 1875 | 1934 | 1993 | 1698 | 1757 | 1816 |
| 71282-10 | 226 | 1052 | 1878 | 1937 | 1996 | 1701 | 1760 | 1819 |
| 71282-14 | 230 | 1056 | 1882 | 1941 | 2000 | 1705 | 1764 | 1823 |
| 71282-18 | 234 | 1060 | 1886 | 1945 | 2004 | 1709 | 1768 | 1827 |

### Methods

Human Subjects. All donors were children between the ages of 5 months and 18 years old. Blood samples were collected with consent on June 24^{th} and August 18^{th} of 2021 and then PBMCs were isolated upon receipt. PBMCs were pooled into either group 71281 (no known history of SARS-CoV-2 infection or vaccination) or group 71282 (known history of infection or vaccination) based on self-reporting. Additional details on the age and sex distribution of both groups is further detailed in FIGS. 5A-5B. Additional information about these donors is unavailable.

Cell Lines. A variety of cell lines were used for different assays in this study. Expi293F mammalian cells (ThermoFisher, A14527) were maintained in FreeStyle F17 expression medium supplemented with a final concentration of 0.1% Pluronic Acid F-68 and 4 mM L-Glutamine. ExpiCHO cells (ThermoFisher, A29127) were maintained in ExpiCHO Expression medium (ThermoFisher, A2910002). Cells were cultured at 37 C with 8% CO₂ saturation while shaking. Vero E6 cells (ATCC, CRL-1586) and all HEK293T cell lines (ATCC, CRL-3216; ATCC, CRL-11268; BEI, NR-52511) were maintained in Dulbecco's minimal essential medium (DMEM) supplemented with 10 mM HEPES pH 7.3, 1× non-essential amino acids, 1 mM sodium pyruvate, 100 U/mL of penicillin-streptomycin, and 10% fetal bovine serum and grown in 37 C with 5% CO₂. Authentication analysis was not performed on the cell lines used.

Viruses. The generation of a replication-competent VSV expressing SARS-CoV-2 S protein with a 21 amino-acid C-terminal deletion that replaces the VSV G protein (VSV-SARS-CoV-2) was described previously (Case et al., 2020b). The S protein-expressing VSV virus was propagated in MA104 cell culture monolayers (African green monkey, ATCC CRL-2378.1) as described previously (Case et al., 2020b), and viral stocks were titrated on Vero E6 cell monolayer cultures. VSV plaques were visualized using neutral red staining. All work with infectious SARS-CoV-2 was performed in Institutional Biosafety Committee approved BSL3 and A-BSL3 facilities at Washington University School of Medicine using appropriate positive pressure air respirators and protective equipment.

Blood sample collection and preparation. Blood was collected for diagnostic testing into K2EDTA-coated Becton-Dickenson tubes and mixed to prevent coagulation. Blood was refrigerated after routine hematology testing was performed, until selection for the study (within 48 hours). Blood samples were selected and delivered to the Georgiev lab and PBMCs were isolated immediately upon receipt. Use of the leftover diagnostic peripheral blood samples is covered by Vanderbilt University Medical Center IRB approval (#210733).

PBMC isolation. Upon receiving samples from VUMC Diagnostic Laboratories, whole blood was separated and pooled based on the parameters described in the results section. Pooled blood samples were diluted with 3 parts DPBS. 12.5 mL of Ficoll was added to 50 mL conical tubes and then the diluted blood (up to 30 mL per 50 mL conical) was pipetted to mix before carefully layering it on top of the Ficoll. Without disrupting the Ficoll/Blood interface, tubes were centrifuged at 400 g for 30 minutes. The buffy coat was carefully removed and transferred to a new 50 mL conical up to a maximum of 25 mL per tube. Tubes were then topped to the 50 mL mark with DPBS and inverted to mix. Tubes were again centrifuged at 400 g. For each tube, the pellets were resuspended in ACK lysing buffer and incubated for 1 minute at room temperature. Resuspended pellets from the same pooled samples were combined and then cells were counted. Tubes were filled up to 50 mL with DPBS and centrifuged at 200 g for 5 minutes at room temperature. Supernatants were decanted and pellets were resuspended in CryoStor® Freeze Media in aliquots of 1 mL each between 10 and 100 million cells/mL. Isolated PBMCs were subject to slow freezing and then placed into liquid nitrogen for storage.

Some PBMCs were isolated using the EasySep™ Direct Human PBMC Isolation Kit (Stemcell) according to kit instructions. In short, EDTA was added to pooled blood samples, mixed with the isolation cocktail, and incubated. RapidSpheres™ were then added to the sample and mixed, followed by incubation of the tube inside of the “The Big Easy” magnet (Cat #18001). The enriched cell suspension was then poured into a new tube, followed by mixing with more RapidSpheres™. Cell mixture was incubated in the magnet for a 2^{nd} time for additional separation. The enriched cell suspension was poured into a new tube and the process was repeated for a third time. Isolated cells were poured into a new tube and subsequently counted and frozen for use as detailed above.

Antigen expression and purification. An assortment of recombinant soluble protein antigens was used in the LIBRA-seq experiment and assays. All Expi293F cells were cultured at 8% CO₂ saturation and 37° C. with shaking in FreeStyle F17 expression media (Thermo Fisher) supplemented to a final concentration of 0.1% Pluronic Acid F-68 and 4 mM L-glutamine.

Plasmids were transiently transfected in Expi293F cells using polyethylenimine or ExpiFectamine™ transfection reagent (Thermo Fisher Scientific) and encoded the following: residues 1-1208 of the SARS-CoV-2 spike with a mutated S1/S2 cleavage site, proline substitutions at positions 817, 892, 899, 942, 986 and 987, and a C-terminal T4-fibritin trimerization motif, an 8× HisTag, and a TwinStrepTag (SARS-CoV-2 S Hexapro (HP)); 1-1208 of the SARS-CoV-2 spike with a mutated S1/S2 cleavage site, proline substitutions at positions 817, 892, 899, 942, 986 and 987, as well as mutations L18F, D80A, L242-244L del, R246I, K417N, E484K, N501Y, and a C-terminal T4-fibritin trimerization motif, an 8×HisTag, and a TwinStrepTag (SARS-CoV-2 spike HP Beta); 1-1208 of the SARS-CoV-2 spike with a mutated S1/S2 cleavage site, proline substitutions at positions 817, 892, 899, 942, 986 and 987, as well as mutations 69-70del, Y144del, N501Y, A570D, P681H, and a C-terminal T4-fibritin trimerization motif, an 8× HisTag, and a TwinStrepTag (SARS-CoV-2 spike HP Alpha); residues 1-1190 of the SARS-CoV spike with proline substitutions at positions 968 and 969, and a C-terminal T4-fibritin trimerization motif, an 8× HisTag, and a TwinStrepTag (SARS-CoV S-2P); residues 1-1291 of the MERS-CoV spike with a mutated S1/S2 cleavage site, proline substitutions at positions 1060 and 1061, and a C-terminal T4-fibritin trimerization motif, an AviTag, an 8× HisTag, and a TwinStrepTag (MERS-CoV S-2P Avi); residues 1-1278 of the HCoV-OC43 spike with proline substitutions at positions 1070 and 1071, and a C-terminal T4-fibritin trimerization motif, an 8× HisTag, and a TwinStrepTag (HCoV-OC43 S-2P); residues 1-1277 of the HCoV-HKU1 spike with a mutated S1/S2 cleavage site, proline substitutions at positions 1067 and 1068, and a C-terminal T4-fibritin trimerization motif, an 8× HisTag, and a TwinStrepTag (HCoV-HKU1 S-2P); 1-1208 of the SARS-CoV-2 spike with a mutated S1/S2 cleavage site, proline substitutions at positions 817, 892, 899, 942, 986 and 987, as well as mutations T19R, de1157/158, L452R, T478K, D614G, P681R, D950N, and a C-terminal T4-fibritin trimerization motif, Avitag, HRV3C, 8× HisTag, and a TwinStrepTag (SARS-CoV-2 Delta S HP); 1-1208 of the SARS-CoV-2 spike with a mutated S1/S2 cleavage site, proline substitutions at positions 817, 892, 899, 942, 986 and 987, as well as mutations A67V, de169/70, T95I, G142D, de1143/145, del11, L212I, G339D, S371L, S373P, S375F, S477N, T478K, E484A, Q493R, Q496S, Q498R, N501Y, Y505H, T547K, D614G, H655Y, N679K, P681H, N764K, D796Y, N856K, Q954H, N969K, L981F, and a C-terminal T4-fibritin trimerization motif, Avitag, HRV3C, 8× HisTag, and a TwinStrepTag (SARS-CoV-2 Omicron BA.1 S HP); 1-1208 of the SARS-CoV-2 spike with a mutated S1/S2 cleavage site, proline substitutions at positions 817, 892, 899, 942, 986 and 987, as well as mutations T19I, Del24-26, G142D, V213G, G339D, S371F, S373P, S375F, T376A, D405N, R408S, K417N, N440K, S477N, T478K, E484A, Q493R, Q498R, N501Y, Y505H, D614G, H655Y, N679K, P681H, N764K, D796Y, Q954H, N969K, and a C-terminal T4-fibritin trimerization motif, Avitag, HRV3C, 8× HisTag, and a TwinStrepTag (SARS-CoV-2 Omicron BA.2 S HP); 1-1204 of the RaTG13 spike (ENA QHR63300.2) with proline substitutions at positions 982 and 983, and a C-terminal T4-fibritin trimerization motif, Avitag, HRV3C, 8× HisTag, and a TwinStrepTag (RaTG13 S-2P); 1-1194 of the LYRa11 (ENA AHX37558.1) spike with proline substitutions at positions 972 and 973, and a C-terminal T4-fibritin trimerization motif, Avitag, HRV3C, 8× HisTag, and a TwinStrepTag (LYRa11 S-2P); 1-1190 of the Civet-007/2004 spike (ENA AAU04646.1) with proline substitutions at positions 968 and 969, and a C-terminal T4-fibritin trimerization motif, Avitag, HRV3C, 8× HisTag, and a TwinStrepTag (Civet-007-2004 S-2P); 1-1387 of the CCoV-HuPn-2018 spike (Vlasova, 2022) with proline substitutions at positions 1140 and 1141, and a C-terminal T4-fibritin trimerization motif, Avitag, HRV3C, 8× HisTag, and a TwinStrepTag (CCoV-HuPn-2018 S-2P). All coronavirus spike supernatants were collected 5-7 days post transfection, sterile filtered, and purified over a StrepTrap XT column (Cytiva Life Sciences). Purified proteins were further purified using a size exclusion Superose6 Increase column (Cytiva Life Sciences). For LIBRA-seq, the purified antigens were then biotinylated with the EZ-Link Sulfo-NHS-Biotin (Thermo Fisher Scientific) using a 50:1 biotin to protein molar ratio for calculations.

Recombinant HIV-1 gp140 SOSIP BG505 N332T trimer (Georgiev, 2015) was cultured in Expi293F cells and transfected in the same method as above. The clarified supernatant was run over an affinity column of agarose-bound Galanthus nivalis lectin (GNA, Snowdrop) slowly at 4° C. The column was washed with 1×PBS and bound protein was eluted with 1M methyl-a-D-mannopyranoside in PBS. The protein eluate was buffer exchanged into 1×PBS and then purified by size exclusion chromatography using a Superdex 200 Increase 10/300 GL Sizing column on the AKTA FPLC system (GE Life Sciences). The fractions of purified protein were analyzed by SDS-PAGE and binding was confirmed using ELISA with known antibodies.

The recombinant HA protein (A/New Caledonia/20/99 H1N1 GenBank ACF41878 (NC99)) was produced using Expi 293F cells and the Expifectamine 293 transfection reagent. The protein contains the HA ectodomain with a point mutation at the sialic acid-binding site (Y98F), T4 fibritin foldon trimerization domain, AviTag, and hexahistidine-tag. The cells were cultured for 4-5 days, then the supernatant was harvested and sterile filtered. The pH and NaCl concentration were adjusted by adding 1M Tris-HCl (pH 7.5) and 5M NaCl to 50 mM and 500 mM, respectively. The supernatant was then mixed with Ni Sepharose excel resin (GE Healthcare) to capture the hexahistidine tag. The resin was isolated in a column by gravity and the captured HA protein was eluted by a Tris-NaCl (pH 7.5) buffer containing 300 mM imidazole. The eluted protein was further purified by size exclusion chromatography using a HiLoad 16/60 Superdex 200 column (GE Healthcare). Fractions containing the appropriately sized HA protein were concentrated, analyzed by SDS-PAGE, and tested for antigenicity by ELISA using known antibodies. The proteins were then stored at −80 C until use.

The SARS-CoV-2 S1, SARS-CoV-2 S2, SARS-CoV-2 RBD, and SARS-CoV-2 NTD subdomains as well as recombinant SARS-CoV-2 Omicron BA.4/5 S HP, and HCoV-229E S were purchased from Sino Biological.

Oligonucleotide barcodes. Oligonucleotides composed of a 15 bp antigen barcode were used, a sequence designed for annealing to the template switch oligo on the IOX bead-delivered oligos, and contains truncated TruSeq small RNA read 1 sequences in the following structure: 5′-CCTTGGCACCCGAGAATTCCANNNNNCCCATATAAGA*A*A-3′ (SEQ ID NO: 2007), where Ns represent the antigen barcode. For each antigen the following barcode sequences were used: SARS-CoV-2 S HP (ACAATTTGTCTGCGA) (SEQ ID NO: 2008), SARS-CoV-2 Alpha S HP (GGTAGCCCTAGAGTA) (SEQ ID NO: 2009), SARS-CoV-2 Beta S HP (ATTCGCCTTACGCAA) (SEQ ID NO: 2010), SARS-CoV S-2P (GACCTCATTGTGAAT) (SEQ ID NO: 2011), LYRa11 S-2P (AGACTAATAGCTGAC) (SEQ ID NO: 2012), RaTG13 S-2P (TGTGTATTCCCTTGT) (SEQ ID NO: 2013), Civet-007-2004 S-2P (GACAAGTGATCTGCA) (SEQ ID NO: 2014), MERS S-2P (AACCTTCCGTCTAAG) (SEQ ID NO: 2015), HCoV-OC43 S-2P (TAACTCAGGGCCTAT) (SEQ ID NO: 2016), HCoV-HKU1-S-2P (CAGCCCACTGCAATA) (SEQ ID NO: 2017), HCoV-229E S (TACGCCTATAACTTG) (SEQ ID NO: 2018), BG505 gp140 SOSIP N332T (AACCCACCGTTGTTA) (SEQ ID NO: 2019), HA NC99 (GCTCCTTTACACGTA) (SEQ ID NO: 2020). Oligos were ordered from Sigma-Aldrich and IDT with a 5′ amino modification and HPLC purified.

Labeling antigens with DNA oligonucleotide barcodes. For each antigen described above, the unique DNA barcodes were directly conjugated to the antigen using a SoluLINK Protein-Oligonucleotide Conjugation kit (TriLink, S-9011) according to kit protocol. In short, the oligonucleotide and protein were desalted, modified the amino-oligonucleotide with the 4FB cross-linker, and modified the biotinylated antigen with S-HyNic. Afterwards, the 4FB-oligonucleotide and the HyNic-antigen were mixed to form a stable bond between the protein and the oligonucleotide. The antigen-oligonucleotide concentrations were then determined using a bicinchoninic acid (BCA) assay, and the HyNic molar substitution ratios of each antigen-oligonucleotide conjugate was determined using a NanoDrop according to SoluLINK protocol instructions. Excess oligonucleotide was removed from the protein-oligonucleotide conjugates using an AKTA FPLC and were subsequently verified using SDS-PAGE and silver stain. The optimal amounts of antigen-oligonucleotide conjugates to be used in antigen-specific B cell sorting were then determined through flow cytometry titration experiments on cell lines expressing BCRs of known specificities.

Antigen specific B cell sorting. To start, PBMCs were thawed, washed, and counted. Viability was evaluated using Trypan Blue. The cells were then washed with a solution of DPBS supplemented with 0.1% Bovine serum albumin (BSA). Afterwards, the cells were resuspended in DPBS-BSA and stained with cell markers: Ghost Red 780 for viability, CD14-APC-Cy7, CD3-FITC, CD19-BV711 and IgG-PE-Cy5. Additionally, antigen-oligo conjugates were added to the stain. After a 30-minute incubation in the dark at room temperature, the cells were washed again three times with DPBS-BSA at 300 g for five minutes. Then, the cells were incubated for 15 minutes at room temperature with Streptavidin-PE to label cells with bound antigen. The cells were again washed three times with DPBS-BSA, resuspended in DPBS, and sorted by FACS. Antigen positive B cells were bulk sorted and delivered to the Vanderbilt Technologies for Advanced Genomics (VANTAGE) sequencing core at an appropriate target concentration for 10× Genomics library preparation and subsequent sequencing. FACS data were analyzed using FlowJo.

Sample and library preparation for sequencing. Single-cell suspensions were processed using the Chromium Controller microfluidics device (10× Genomics) and the B cell Single Cell V(D)J solution as per the manufacturer's instructions. The aim was to capture 10,000 B cells per ⅛ 10× cassette. Slight modifications were made to intercept, amplify, and purify the antigen barcode libraries, as previously described. (Setliff, 2019)

Sequence processing and bioinformatics analysis. Our established pipeline was followed, which takes paired-end FASTQ files of oligonucleotide libraries as input, to process and annotate reads for cell barcodes, unique molecular identifiers (UMIs) and antigen barcodes, resulting in a cell barcode-antigen barcode UMI count matrix. (Setliff, 2019) B cell receptor contigs were processed using CellRanger (10× Genomics) and GRCh38 as reference, while the antigen barcode libraries were also processed using CellRanger (10× Genomics). The cell barcodes that overlapped between the two libraries formed the basis of the subsequent analysis. Cell barcodes that had only non-functional heavy chain sequences as well as cells with multiple functional heavy chain sequences and/or multiple functional light chain sequences, were eliminated, reasoning that these may be multiplets. B cell receptor contigs (filtered_contigs.fasta file output by CellRanger, 10× Genomics) were aligned to IMGT reference genes using HighV-Quest. (Alamyar, 2012) The output of HighV-Quest was parsed using ChangeO (Gupta, 2015), and combined with an antigen barcode UMI count matrix. Finally, the LIBRA-seq score for each antigen in the library was determined for every cell as previously described. (Setliff, 2019)

High-throughput antibody microscale expression and purification. For high-throughput production of recombinant antibodies, a microscale approach was employed. For antibody expression, microscale transfection was performed (~1 ml per antibody) with CHO cell cultures using the Gibco ExpiCHO Expression System and a protocol for deep 96-well blocks (Thermo Fisher Scientific). Briefly, synthesized antibody-encoding DNA (~2 μg per transfection) was added to OptiPro serum-free medium (OptiPro SFM), incubated with ExpiFectamine CHO Reagent, and added to 800 μl of ExpiCHO cell cultures in deep 96-well blocks using a ViaFlo384 liquid handler (Integra Biosciences). The plates were incubated on an orbital shaker at 1,000 r.p.m. with an orbital diameter of 3 mm at 37° C. in 8% CO2. The next day after transfection, ExpiFectamine CHO Enhancer and ExpiCHO Feed reagents (Thermo Fisher Scientific) were added to the cells, followed by 4 more days of incubation. Culture supernatants were collected after centrifuging the blocks at 450 g for 5 minutes and were stored at 4° C. until use. For high-throughput microscale antibody purification, fritted deep-well plates were used containing 25 μl of settled Protein G resin (GE Healthcare Life Sciences) per well. Clarified culture supernatants were incubated with protein G resin for antibody capturing, washed with PBS using a 96-well plate manifold base (Qiagen) connected to the vacuum and eluted into 96-well PCR plates using 86 μl of 0.1 M glycine-HCl buffer, pH 2.7. Purified antibodies were then neutralized with 14 μl of 1 M Tris-HCl pH 8.0 and buffer exchanged into PBS using Zeba Spin Desalting plates (Thermo Fisher Scientific). Purified antibodies were then stored at 4° C. until use.

Antibody expression and purification. Variable heavy and light genes were inserted into custom plasmids that encode the constant region for the human IgG1 heavy chain and respective lambda and kappa light chains (pTwist CMV BetaGlobin WPRE Neo vector, Twist Bioscience). The antibodies were expressed in Expi293F cells by co-transfecting heavy chain and light chain expressing plasmids using polyethylenimine or Expifectamine transfection reagent, and the cells were cultured for 4-5 days. These cells were maintained as previously described in the antigen purification methods. Cultures were harvested, centrifuged and supernatant was 0.45 μm filtered with Nalgene Rapid Flow Disposable Filter Units with PES membrane. The filtered supernatant was run over a column containing Protein A agarose resin that was equilibrated with PBS. The column was washed with PBS, and then the antibodies were eluted with 100 mM Glycine HCl at 2.7 pH directly into a 1:10 volume of 1M Tris-HCl pH 8.0. Eluted antibodies were buffer exchanged into PBS using Amicon Ultra centrifugal filter units, centrifuging and topping off three times with PBS, and finally concentrated. The antibodies were analyzed by SDS-PAGE. Antibody plasmids were sequenced to confirm the expected heavy and light chain match.

ELISA. To evaluate the binding of the expressed antibodies, soluble purified antigen was plated at a concentration of 2 μg/mL and incubated overnight at 4° C. The next day, the plates were washed three times with a PBS solution containing 0.05% Tween-20 (PBS-T) and then coated with 5% milk powder in PBS-T. The plates were incubated for one hour at room temperature and then washed three times with PBS-T. The primary antibodies were diluted in 1% milk in PBS-T, starting at a concentration of 10 μg/mL with a serial 1:5 or 1:10 dilution and then added to the plate. The plates were incubated for an additional hour at room temperature and then washed three times with PBS-T. The secondary antibody, goat anti-human IgG conjugated to peroxidase, was added at a dilution of 1:10,000 in 1% milk in PBS-T to the plates, which were incubated for one hour at room temperature. The plates were washed three times with PBS-T and then developed by adding TMB substrate to the plates. The plates were incubated for ten minutes at room temperature, and the reaction was stopped with 1N sulfuric acid. Plates were read at 450 nm. The data is shown as one representative biological replicate with the mean±SEM for one ELISA experiment. The ELISAs were repeated 2 or more times. The area under the curve (AUC) was calculated using GraphPad Prism 9.5.0.

ACE2 competition ELISA. 96-well plates were coated with 2 μg/mL of purified recombinant SARS-CoV-2 spike protein, and left to incubate overnight at 4° C. The following day, the plates were washed three times with a PBS solution containing 0.05% Tween-20 (PBS-T) and then coated with 5% nonfat dry milk in PBS-T. The plates were incubated for one hour at room temperature and then washed three times with PBS-T. Next, purified antibodies were diluted in 1% nonfat dry milk in PBS-T at a concentration of 10 μg/mL and added to the wells in triplicate, followed by incubation for an additional hour at room temperature. Without washing, recombinant human ACE2 protein with a mouse Fc tag was added to the wells for a final concentration of 0.4 μg/mL of ACE2 and incubated for 40 minutes at room temperature. The plates were washed three times with PBS-T and bound ACE2 was detected using HRP-conjugated anti-mouse Fc antibody and TMB substrate. The plates were incubated for ten minutes at room temperature, and then the reaction was stopped with 1N sulfuric acid. Plates were read at 450 nm. ACE2 binding without antibody served as a control. The experiment was done in biological replicate and technical triplicates.

Antibody competition ELISA. Antibody competition ELISAs were performed similarly to the regular binding ELISA protocol described above, with some alterations. After antigen coating and blocking, 25 μL of serially diluted non-biotinylated competitor antibody was added to each well at a concentration of 100, 10, 1, and 0.1 μg/mL, and incubated at 37° C. for 10 minutes. Then, without washing, 75 μL of biotinylated antibody (final well concentration of 0.1 μg/mL) was added and incubated at 37° C. for 1 hour. After washing three times with PBS-T, streptavidin-HRP was added at a dilution of 1:10,000 in 1% milk in PBS-T and incubated for 1 hour at room temperature. The plates were washed, and substrate and sulfuric acid were added as described earlier. ELISAs were repeated at least 2 times. Data is shown as the percentage decrease in binding relative to no competitor.

Real-time cell analysis (RTCA) neutralization assay. To determine neutralizing activity of IgG, Fab, or F(ab′)2 proteins, real-time cell analysis (RTCA) assays were used on an xCELLigence RTCA MP Analyzer (ACEA. Biosciences Inc.) that measures virus-induced cvtopathic effect (CPE) (Suryadevara, 2022). Briefly, 50 μL of cell culture medium (DMEM supplemented with 2% FBS) was added to each well of a 96-well E-plate using a ViaFlo384 liquid handler (Integra Biosciences) to obtain background reading. A suspension of 18,000 Vero-E6 cells in 50 μL of cell culture medium was seeded in each well, and the plate was placed on the analyzer. Measurements were taken automatically every 15 min, and the sensograms were visualized using RTCA software version 2.1.0 (ACEA Biosciences Inc). VSV-SARS-CoV-2 (0.01 MO, ~120 PFU1 per well) was mixed 1:1 with a dilution of mAb in a total volume of 100 μL using DMEM supplemented with 2% FBS as a diluent and incubated for 1 h at 37° C. in 5% CO2. At 16 h after seeding the cells, the virus-mAb mixtures were added in replicates to the cells in 96-well E-plates. Triplicate wells containing virus only (maximal CPE in the absence of mAb) and wells containing only Vero cells in medium (no-CPE wells) were included as controls. Plates were measured continuously (every 15 min) for 48 h to assess virus neutralization. Normalized cellular index (CI) values at the endpoint (48 h after incubation with the virus) were determined using the RTCA software version 2.1.0 (ACEA Biosciences Inc.). Results are expressed as percent neutralization in a presence of respective mAb relative to control wells with no CPE minus CI values from control wells with maximum CPE. RTCA IC50 values were determined by nonlinear regression analysis using Prism software.

SARS-CoV-2 VSV-G virus production. The generation of a replication-competent VSV expressing SARS-CoV-2 S protein with a 21 amino-acid C-terminal deletion that replaces the VSV G protein (VSV-SARS-CoV-2) was described previously. (Case, 2020) The S protein-expressing VSV virus was propagated in MA104 cell culture monolayers (African green monkey, ATCC CRL-2378.1) as described previously (Case, 2020), and viral stocks were titrated on Vero E6 cell monolayer cultures. VSV plaques were visualized using neutral red staining. All work with infectious SARS-CoV-2 was performed in Institutional Biosafety Committee approved BSL3 and A-BSL3 facilities at Washington University School of Medicine using appropriate positive pressure air respirators and protective equipment.

Pseudovirus production and neutralization assays. Lentiviral pseudovirus production and neutralizations were done in one of two different ways: (1) For the pseudoviruses D614G, BA.2, BA. 5, BA.4.6, BA.2.75.2, XBB, BQ.1.1, and XBB.1.5: HEK293T cells and HEK293T-hACE2 (stably expressing human ACE2) cells were cultured in Dulbecco's Modified Eagle Medium (high glucose, with sodium pyruvate) supplemented with 10% fetal bovine serum, 100 units/ml Penicillin, and 100 μg/ml Streptomycin. Cultures were maintained in a humidified 37 C incubator (5% CO2). SARS-CoV-2 spike-pseudotyped lentiviruses were generated by the co-transfection of HEK293T cells with a spike-encoding plasmid (with a 19 amino acid C-terminal truncation), a lentiviral packaging plasmid (Addgene #8455), and a transfer plasmid encoding firefly luciferase (Addgene #170674) using polyethylenimine (PEI). Growth media was replaced 12-16 h after transfection, and pseudotyped viruses were harvested from the supernatant at 48- and 72-h post transfection, clarified by centrifugation, and stored at −80 C. Pseudotyped viruses previously titrated to generate ~100,000 relative light units (RLUs) were incubated with serial 3-fold dilutions of antibody for 60 min at 37 C, and then ~10,000 HEK293T-hACE2 cells were added to each well. Plates were incubated at 37 C for 44-48 h and luminescence was then measured using Bright-Glo (Promega) on a GM-2000 luminometer (Promega) per the manufacturer's instructions. Neutralization was calculated relative to the mean of eight control wells infected in the absence of antibody and fit using a four-parameter logistic curve in Prism v9 (GraphPad Software).

(2) For the pseudovirus BA.2.75+R346T: Lentiviral pseudoviruses bearing the S glycoproteins of SARS-CoV-2 variants were generated using a previously described protocol. (Crawford, 2020) Briefly, HEK-293T/17 cells (ATCC, cat. CRL-11268) were seeded in a culture flask at a density that would give approximately 50-70% confluency on the day of transfection. The morning of the transfection, media was changed to fresh DMEM supplemented with sodium pyruvate, penicillin/streptomycin 25 mM HEPES, and 10% (vol/vol) FBS. To generate lentiviral-based reporter pseudoviruses, cells were co-transfected with a pHAGE-CMV-Luc2-IRES-ZsGreen-W plasmid encoding the lentiviral genome (BEI Resources, cat. NR-52516), the lentiviral packaging plasmids HDM-Hgpm2 (BEI Resources, cat. NR-52517), HDM-tatlb (BEI Resources, cat. NR-52518), and pRC-CMV-Rev (BEI Resources, cat. NR-52519), and a plasmid encoding the SARS-CoV-2 S gene with a 21 amino acid deletion. Approximately 16 to 18 h later, media was removed and replaced with fresh DMEM supplemented with sodium pyruvate, penicillin/streptomycin 25 mM HEPES, and 2% (vol/vol) FBS. Approximately 48 h after transfection, the supernatant was collected from each flask, clarified by centrifugation, and filtered through a 0.2 μm filter. Aliquots of pseudovirus stocks were prepared and stored at −80° C.

Lentivirus-based pseudoviral neutralization assays were executed based on a previous described protocol (Crawford, 2020). One day prior to the assay, HEK-293T cells transduced to stably express human ACE2 (293T-hACE2 cells, BEI Resources NR-52511) were seeded at a density of 1.25×104 cells per well in DMEM+10% FBS onto 96-well tissue culture plates that had previously been coated with poly-D-lysine (ThermoFisher Scientific, cat. A3890401) The day of the assay, monoclonal antibodies were diluted in DMEM+2% FBS using a four-fold dilution series (with each antibody in technical duplicate) in a 96-well polypropylene microtiter plate and incubated with pseudovirus for 1 h at 37° C. in the presence of a final concentration of 5 μg/mL polybrene (EMD Millipore). After the 1 h incubation, pseudovirus-mAb mixtures were added to 293T-hACE2 monolayers. Plates were incubated at 37° C. for 48-60 h, at which point cells were lysed using the Bright-Glo Luciferase Assay System (Promega) and luciferase activity was quantified using a CLARIOStar plate reader (BMG LabTech). The luminescence signal from wells in each plate where no pseudovirus or antibody was added was averaged and subtracted from each value, after which the percent infection of each well was determined relative to the average of pseudovirus only control wells present in each plate. IC50 values were determined by nonlinear regression using Prism v.9.5 (GraphPad) using a four-parameter [inhibitor] vs response curve fit with top and bottom values constrained to 100 and 0, respectively. Each neutralization assay was repeated twice.

Public antibody analysis. For generation of plots for each child antibody in comparison with previously published adult antibodies with respective to percent CDRH3 (x-axis) and percent CDRL3 (y-axis) identity, CovAbDab database was used. (Raybould, 2021) Each dot is colored according to V-gene usage: blue if both the VH and VL of the given child and adult antibodies match, orange if only the VH match, purple if only the VL match, and grey if neither match but at least either one of the CDRH3 or CDRL3 have >=50% sequence identity for the child vs. adult antibody.

Sequences and gene annotations for antibodies in clinical use were manually curated from IMGT/3Dstructure-DB (Kaas, 2004) and compared to the SARS-CoV-2 antibodies from children using a custom Python script. Heavy chain CDR3 identity between each antibody pair was defined by calculating the Levenshtein distance between the amino acid sequences and dividing by the length of the longer CDR3 region.

Negative stain Electron microscopy (nsEM) sample and grid preparation, imaging and processing of spike-Fab complexes. To perform electron microscopy imaging, Fabs were produced by digesting recombinant chromatography-purified IgGs using resin-irnmobilized cysteine protease enzyme (FabALACTICA, Genovis). The digestion occurred in 100 mM sodium phosphate and 150 mM NaCl pH 7.2 (PBS) for around 16 h at ambient temperature. To remove cleaved Fe from intact IgG, the digestion mix was incubated with CaptureSelect Fc resin (Genovis) for 30 min at ambient temperature in PBS buffer. Spike protein were incubated with the Fab at an Fab:S protein molar ratio of 4:1 for about 1 hour at ambient temperature, and approximately 3 μL of the sample at concentrations of about 10-15 μg/mL was applied to a glow-discharged grid with continuous carbon film on 400 square mesh copper electron microscopy grids (Electron Microscopy Sciences). The grids were stained with 2% uranyl formate. (Ohi, 2004) Images were recorded on a Gatan US4000 4 k×4 k CCD camera using an TF20 transmission electron microscope (TFS) operated at 200 keV and control with Serial EM. (Mastronarde, 2003) All images were taken at 50,000× magnification with a pixel size of 2.18 Å/pixel in low-dose mode at a defocus of 1.5 to 1.8 μm. The total dose for the micrographs was around 30e− per N2. Image processing was performed using the cryoSPARC software package. (Punjani, 2017) Images were imported, CTF-estimated and particles were picked. The particles were extracted with a box size of 256 pixels and binned to 128 pixels (4.36 Å/pixel). 2D class averages were performed and good classes selected for ab initio model and NU refinement without symmetry with final resolution of ~20 Å. Model docking to the EM map was done in ChimeraX. (Pettersen, 2021) For spike protein model (PDB:7XIW) was used and PDB:12E8 was used for the Fab. ChimeraX software was used to make all figures, and the EM map has been deposited into EMDB (EMD-41075, EMD-41076) Quantification And Statistical Analysis. The ELISA error bars (±standard error of the mean) were calculated using Graphpad Prism 9.5.0. Mean±SEM or mean±SD were determined for continuous variables as noted. Technical and biological replicates are described in the figure legends. Details of the statistical analysis can be found in the main text and respective figure captions.

### REFERENCES

Ruf, B. R., and Knuf, M. (2014). The burden of seasonal and pandemic influenza in infants and children. European Journal of Pediatrics 173, 265-276. 10.1007/s00431-013-2023-6.
Shafagati, N., and Williams, J. (2018). Human metapneumovirus—what we know now. F1000Research 7, 135. 10.12688/f1000research.12625.1.
Shi, T., McAllister, D. A., O'Brien, K. L., Simoes, E. A. F., Madhi, S. A., Gessner, B. D., Polack, F. P., Balsells, E., Acacio, S., Aguayo, C., et al. (2017). Global, regional, and national disease burden estimates of acute lower respiratory infections due to respiratory syncytial virus in young children in 2015: a systematic review and modelling study. The Lancet 390, 946-958. 10.1016/s0140-6736(17)30938-8.
Zimmermann, P., and Curtis, N. (2021). Why is COVID-19 less severe in children? A review of the proposed mechanisms underlying the age-related difference in severity of SARS-CoV-2 infections. Archives of Disease in Childhood 106, 429-439. 10.1136/archdischild-2020-320338.
Mehta, N. S., Mytton, O. T., Mullins, E. W. S., Fowler, T. A., Falconer, C. L., Murphy, O. B., Langenberg, C., Jayatunga, W. J. P., Eddy, D. H., and Nguyen-Van-Tam, J. S. (2020). SARS-CoV-2 (COVID-19): What Do We Know About Children?A Systematic Review. Clinical Infectious Diseases 71, 2469-2479. 10.1093/cid/ciaa556.
Chou, J., Thomas, P. G., and Randolph, A. G. (2022). Immunology of SARS-CoV-2 infection in children. Nature Immunology 23, 177-185. 10.1038/s41590-021-01123-9.
Mathew, D., Giles, J. R., Baxter, A. E., Oldridge, D. A., Greenplate, A. R., Wu, J. E., Alanio, C., Kuri-Cervantes, L., Pampena, M. B., D'Andrea, K., et al. (2020). Deep immune profiling of COVID-19 patients reveals distinct immunotypes with therapeutic implications. Science 369. 10.1126/science.abc8511.
Kramer, K. J., Wilfong, E. M., Voss, K., Barone, S. M., Shiakolas, A. R., Raju, N., Roe, C. E., Suryadevara, N., Walker, L. M., Wall, S. C., et al. (2022). Single-cell profiling of the antigen-specific response to BNT162b2 SARS-CoV-2 RNA vaccine. Nat Commun 13, 3466. 10.1038/s41467-022-31142-5.
Diamond, M. S., and Kanneganti, T.-D. (2022). Innate immunity: the first line of defense against SARS-CoV-2. Nature Immunology 23, 165-176. 10.1038/s41590-021-01091-0.
Cromer, D., Steain, M., Reynaldi, A., Schlub, T. E., Wheatley, A. K., Juno, J. A., Kent, S. J., Triccas, J. A., Khoury, D. S., and Davenport, M. P. (2022). Neutralising antibody titres as predictors of protection against SARS-CoV-2 variants and the impact of boosting: a meta-analysis. The Lancet Microbe 3, e52-e61. 10.1016/s2666-5247(21)00267-6.
Krammer, F. (2020). SARS-CoV-2 vaccines in development. Nature 586, 516-527. 10.1038/s41586-020-2798-3.
Wang, Z., Schmidt, F., Weisblum, Y., Muecksch, F., Barnes, C. O., Finkin, S., Schaefer-Babajew, D., Cipolla, M., Gaebler, C., Lieberman, J. A., et al. (2021). mRNA vaccine-elicited antibodies to SARS-CoV-2 and circulating variants. Nature 592, 616-622. 10.1038/s41586-021-03324-6.
Corbett, K. S., Edwards, D. K., Leist, S. R., Abiona, O. M., Boyoglu-Barnum, S., Gillespie, R. A., Himansu, S., Schafer, A., Ziwawo, C. T., Dipiazza, A. T., et al. (2020). SARS-CoV-2 mRNA vaccine design enabled by prototype pathogen preparedness. Nature 586, 567-571. 10.1038/s41586-020-2622-0.
Khoury, D. S., Cromer, D., Reynaldi, A., Schlub, T. E., Wheatley, A. K., Juno, J. A., Subbarao, K., Kent, S. J., Triccas, J. A., and Davenport, M. P. (2021). Neutralizing antibody levels are highly predictive of immune protection from symptomatic SARS-CoV-2 infection. Nature Medicine 27, 1205-1211. 10.1038/s41591-021-01377-8.
Collier, D. A., Ferreira, I. A. T. M., Kotagiri, P., Datir, R. P., Lim, E. Y., Touizer, E., Meng, B., Abdullahi, A., Baker, S., Dougan, G., et al. (2021). Age-related immune response heterogeneity to SARS-CoV-2 vaccine BNT162b2. Nature 596, 417-422. 10.1038/s41586-021-03739-1.
Cele, S., Jackson, L., Khoury, D. S., Khan, K., Moyo-Gwete, T., Tegally, H., San, J. E., Cromer, D., Scheepers, C., Amoako, D. G., et al. (2022). Omicron extensively but incompletely escapes Pfizer BNT162b2 neutralization. Nature 602, 654-656. 10.1038/s41586-021-04387-1.
Barnes, C. O., West, A. P., Huey-Tubman, K. E., Hoffmann, M. A. G., Sharaf, N. G., Hoffman, P. R., Koranda, N., Gristick, H. B., Gaebler, C., Muecksch, F., et al. (2020). Structures of Human Antibodies Bound to SARS-CoV-2 Spike Reveal Common Epitopes and Recurrent Features of Antibodies. Cell 182, 828-842.e816. 10.1016/j.cell.2020.06.025.
Setliff, I., McDonnell, W. J., Raju, N., Bombardi, R. G., Murji, A. A., Scheepers, C., Ziki, R., Mynhardt, C., Shepherd, B. E., Mamchak, A. A., et al. (2018). Multi-Donor Longitudinal Antibody Repertoire Sequencing Reveals the Existence of Public Antibody Clonotypes in HIV-1 Infection. Cell Host Microbe 23, 845-854 e846. 10.1016/j.chom.2018.05.001.
Chen, E. C., Gilchuk, P., Zost, S. J., Suryadevara, N., Winkler, E. S., Cabel, C. R., Binshtein, E., Chen, R. E., Sutton, R. E., Rodriguez, J., et al. (2021). Convergent antibody responses to the SARS-CoV-2 spike protein in convalescent and vaccinated individuals. Cell Rep 36, 109604. 10.1016/j.celrep.2021.109604.
Tan, T. J. C., Yuan, M., Kuzelka, K., Padron, G. C., Beal, J. R., Chen, X., Wang, Y., Rivera-Cardona, J., Zhu, X., Stadtmueller, B. M., et al. (2021). Sequence signatures of two public antibody clonotypes that bind SARS-CoV-2 receptor binding domain. Nature Communications 12. 10.1038/s41467-021-24123-7.
Zost, S. J., Gilchuk, P., Case, J. B., Binshtein, E., Chen, R. E., Nkolola, J. P., Schafer, A., Reidy, J. X., Trivette, A., Nargi, R. S., et al. (2020). Potently neutralizing and protective human antibodies against SARS-CoV-2. Nature 584, 443-449. 10.1038/s41586-020-2548-6.
Liu, L., Wang, P., Nair, M. S., Yu, J., Rapp, M., Wang, Q., Luo, Y., Chan, J. F., Sahi, V., Figueroa, A., et al. (2020). Potent neutralizing antibodies against multiple epitopes on SARS-CoV-2 spike. Nature 584, 450-456. 10.1038/s41586-020-2571-7.
Pinto, D., Park, Y. J., Beltramello, M., Walls, A. C., Tortorici, M. A., Bianchi, S., Jaconi, S., Culap, K., Zatta, F., De Marco, A., et al. (2020). Cross-neutralization of SARS-CoV-2 by a human monoclonal SARS-CoV antibody. Nature 583, 290-295. 10.1038/s41586-020-2349-y.
Rogers, T. F., Zhao, F., Huang, D., Beutler, N., Burns, A., He, W. T., Limbo, O., Smith, C., Song, G., Woehl, J., et al. (2020). Isolation of potent SARS-CoV-2 neutralizing antibodies and protection from disease in a small animal model. Science 369, 956-963. 10.1126/science.abc7520.
Shi, R., Shan, C., Duan, X., Chen, Z., Liu, P., Song, J., Song, T., Bi, X., Han, C., Wu, L., et al. (2020). A human neutralizing antibody targets the receptor-binding site of SARS-CoV-2. Nature 584, 120-124. 10.1038/s41586-020-2381-y.
Jones, B. E., Brown-Augsburger, P. L., Corbett, K. S., Westendorf, K., Davies, J., Cujec, T. P., Wiethoff, C. M., Blackbourne, J. L., Heinz, B. A., Foster, D., et al. (2020). LY-CoV555, a rapidly isolated potent neutralizing antibody, provides protection in a non-human primate model of SARS-CoV-2 infection. bioRxiv. 10.1101/2020.09.30.318972.
Hansen, J., Baum, A., Pascal, K. E., Russo, V., Giordano, S., Wloga, E., Fulton, B. O., Yan, Y., Koon, K., Patel, K., et al. (2020). Studies in humanized mice and convalescent humans yield a SARS-CoV-2 antibody cocktail. Science 369, 1010-1014. 10.1126/science.abd0827.
Sun, Y., and Ho, M. (2020). Emerging antibody-based therapeutics against SARS-CoV-2 during the global pandemic. Antib Ther 3, 246-256. 10.1093/abt/tbaa025.37
Weisblum, Y., Schmidt, F., Zhang, F., Dasilva, J., Poston, D., Lorenzi, J. C., Muecksch, F., Rutkowska, M., Hoffmann, H.-H., Michailidis, E., et al. (2020). Escape from neutralizing antibodies by SARS-CoV-2 spike protein variants. eLife 9. 10.7554/elife.61312.
Chen, R. E., Zhang, X., Case, J. B., Winkler, E. S., Liu, Y., Vanblargan, L. A., Liu, J., Errico, J. M., Xie, X., Suryadevara, N., et al. (2021). Resistance of SARS-CoV-2 variants to neutralization by monoclonal and serum-derived polyclonal antibodies. Nature Medicine 27, 717-726. 10.1038/s41591-021-01294-w.
Wang, P., Nair, M. S., Liu, L., Iketani, S., Luo, Y., Guo, Y., Wang, M., Yu, J., Zhang, B., Kwong, P. D., et al. (2021). Antibody resistance of SARS-CoV-2 variants B.1.351 and B.1.1.7. Nature 593, 130-135. 10.1038/s41586-021-03398-2.
Wang, Q., Guo, Y., Iketani, S., Nair, M. S., Li, Z., Mohri, H., Wang, M., Yu, J., Bowen, A. D., Chang, J. Y., et al. (2022). Antibody evasion by SARS-CoV-2 Omicron subvariants BA.2.12.1, BA.4 and BA.5. Nature 608, 603-608. 10.1038/s41586-022-05053-w.
Wang, Q., Iketani, S., Li, Z., Liu, L., Guo, Y., Huang, Y., Bowen, A. D., Liu, M., Wang, M., Yu, J., et al. (2023). Alarming antibody evasion properties of rising SARS-CoV-2 BQ and XBB subvariants. Cell 186, 279-286.e278. 10.1016/j.cell.2022.12.018.
Sheward, D. J., Kim, C., Fischbach, J., Sato, K., Muschiol, S., Ehling, R. A., Bjorkstrom, N. K., Hedestam, G. B. K., Reddy, S. T., Albert, J., et al. (2022). Omicron sublineage BA.2.75.2 exhibits extensive escape from neutralising antibodies. The Lancet Infectious Diseases 22, 1538-1540. 10.1016/s1473-3099(22)00663-6.
Cao, Y., Yisimayi, A., Jian, F., Song, W., Xiao, T., Wang, L., Du, S., Wang, J., Li, Q., Chen, X., et al. (2022). BA.2.12.1, BA.4 and BA.5 escape antibodies elicited by Omicron infection. Nature 608, 593-602. 10.1038/s41586-022-04980-y.
Imai, M., Ito, M., Kiso, M., Yamayoshi, S., Uraki, R., Fukushi, S., Watanabe, S., Suzuki, T., Maeda, K., Sakai-Tagawa, Y., et al. (2023). Efficacy of Antiviral Agents against Omicron Subvariants BQ.1.1 and XBB. New England Journal of Medicine 388, 89-91. 10.1056/nejmc2214302.
Shiakolas, A. R., Kramer, K. J., Johnson, N. V., Wall, S. C., Suryadevara, N., Wrapp, D., Periasamy, S., Pilewski, K. A., Raju, N., Nargi, R., et al. (2022). Efficient discovery of SARS-CoV-2-neutralizing antibodies via B cell receptor sequencing and ligand blocking. Nat Biotechnol 40, 1270-1275. 10.1038/s41587-022-01232-2.
Cao, Y., Jian, F., Zhang, Z., Yisimayi, A., Hao, X., Bao, L., Yuan, F., Yu, Y., Du, S., Wang, J., et al. (2022). Rational identification of potent and broad sarbecovirus-neutralizing antibody cocktails from SARS convalescents. Cell Reports 41, 111845. 10.1016/j.celrep.2022.111845.
Astrazeneca (2022). AstraZeneca Data on File—REF-173560.
Dowell, A. C., Butler, M. S., Jinks, E., Tut, G., Lancaster, T., Sylla, P., Begum, J., Bruton, R., Pearce, H., Verma, K., et al. (2022). Children develop robust and sustained cross-reactive spike-specific immune responses to SARS-CoV-2 infection. Nat Immunol 23, 40-49. 10.1038/s41590-021-01089-8.
Fraley, E., Lemaster, C., Banerjee, D., Khanal, S., Selvarangan, R., and Bradley, T. (2021). Cross-reactive antibody immunity against SARS-CoV-2 in children and adults. Cellular & Molecular Immunology 18, 1826-1828. 10.1038/s41423-021-00700-0.
Yang, F., Nielsen, S. C. A., Hoh, R. A., Roltgen, K., Wirz, O. F., Haraguchi, E., Jean, G. H., Lee, J. Y., Pham, T. D., Jackson, K. J. L., et al. (2021). Shared B cell memory to coronaviruses and other pathogens varies in human age groups and tissues. Science 372, 738-741. 10.1126/science.abf6648.
Setliff, I., Shiakolas, A. R., Pilewski, K. A., Murji, A. A., Mapengo, R. E., Janowska, K., Richardson, S., Oosthuysen, C., Raju, N., Ronsard, L., et al. (2019). High-Throughput Mapping of B Cell Receptor Sequences to Antigen Specificity. Cell 179, 1636-1646 e1615. 10.1016/j. cell.2019.11.003.
Case, J. B., Rothlauf, P. W., Chen, R. E., Liu, Z., Zhao, H., Kim, A. S., Bloyet, L. M., Zeng, Q., Tahan, S., Droit, L., et al. (2020). Neutralizing Antibody and Soluble ACE2 Inhibition of a Replication-Competent VSV-SARS-CoV-2 and a Clinical Isolate of SARS-CoV-2. Cell Host Microbe 28, 475-485 e475. 10.1016/j.chom.2020.06.021.
Suryadevara, N., Gilchuk, P., Zost, S. J., Mittal, N., Zhao, L. L., Crowe, J. E., Jr., and Carnahan, R. H. (2022). Real-time cell analysis: A high-throughput approach for testing SARS-CoV-2 antibody neutralization and escape. STAR Protoc 3, 101387. 10.1016/j.xpro.2022.101387.
Zhang, L., Jackson, C. B., Mou, H., Ojha, A., Peng, H., Quinlan, B. D., Rangarajan, E. S., Pan, A., Vanderheiden, A., Suthar, M. S., et al. (2020). SARS-CoV-2 spike-protein D614G mutation increases virion spike density and infectivity. Nature Communications 11. 10.1038/s41467-020-19808-4.
Cao, Y., Jian, F., Wang, J., Yu, Y., Song, W., Yisimayi, A., Wang, J., An, R., Chen, X., Zhang, N., et al. (2022). Imprinted SARS-CoV-2 humoral immunity induces convergent Omicron RBD evolution. Nature. 10.1038/s41586-022-05644-7.
Kim, S. I., Noh, J., Kim, S., Choi, Y., Yoo, D. K., Lee, Y., Lee, H., Jung, J., Kang, C. K., Song, K. H., et al. (2021). Stereotypic neutralizing V(H) antibodies against SARS-CoV-2 spike protein receptor binding domain in patients with COVID-19 and healthy individuals. Sci Transl Med 13. 10.1126/scitranslmed.abd6990.
Cao, Y., Su, B., Guo, X., Sun, W., Deng, Y., Bao, L., Zhu, Q., Zhang, X., Zheng, Y., Geng, C., et al. (2020). Potent Neutralizing Antibodies against SARS-CoV-2 Identified by High-Throughput Single-Cell Sequencing of Convalescent Patients' B Cells. Cell 182, 73-84.e16. 10.1016/j.cell.2020.05.025.
Raybould, M. I. J., Kovaltsuk, A., Marks, C., and Deane, C. M. (2021). CoV-AbDab: the coronavirus antibody database. Bioinformatics 37, 734-735. 10.1093/bioinformatics/btaa739.
Dong, J., Zost, S. J., Greaney, A. J., Starr, T. N., Dingens, A. S., Chen, E. C., Chen, R. E., Case, J. B., Sutton, R. E., Gilchuk, P., et al. (2021). Genetic and structural basis for SARS-CoV-2 variant neutralization by a two-antibody cocktail. Nature Microbiology 6, 1233-1244. 10.1038/s41564-021-00972-2.
Tortorici, M. A., Beltramello, M., Lempp, F. A., Pinto, D., Dang, H. V., Rosen, L. E., McCallum, M., Bowen, J., Minola, A., Jaconi, S., et al. (2020). Ultrapotent human antibodies protect against SARS-CoV-2 challenge via multiple mechanisms. Science 370, 950-957. 10.1126/science.abe3354.
Wang, Y., Yuan, M., Lv, H., Peng, J., Wilson, I. A., and Wu, N. C. (2022). A large-scale systematic survey reveals recurring molecular features of public antibody responses to SARS-CoV-2. Immunity 55, 1105-1117.e1104. 10.1016/j.immuni.2022.03.019.
Chen, E. C., Gilchuk, P., Zost, S. J., Suryadevara, N., Winkler, E. S., Cabel, C. R., Binshtein, E., Chen, R. E., Sutton, R. E., Rodriguez, J., et al. (2021). Convergent antibody responses to the SARS-CoV-2 spike protein in convalescent and vaccinated individuals. Cell reports 36, 109604. https://doi.org/10.1016/j.celrep.2021.109604.
Westendorf, K., Zentelis, S., Wang, L., Foster, D., Vaillancourt, P., Wiggin, M., Lovett, E., Van Der Lee, R., Hendle, J., Pustilnik, A., et al. (2022). LY-CoV1404 (bebtelovimab) potently neutralizes SARS-CoV-2 variants. Cell Reports 39, 110812. 10.1016/j.celrep.2022.110812.
Yuan, M., Wang, Y., Lv, H., Tan, T. J. C., Wilson, I. A., and Wu, N. C. (2022). Molecular analysis of a public cross-neutralizing antibody response to SARS-CoV-2. Cell Reports 41, 111650. 10.1016/j.celrep.2022.111650.
Zhang, Q., Ju, B., Ge, J., Chan, J. F.-W., Cheng, L., Wang, R., Huang, W., Fang, M., Chen, P., Zhou, B., et al. (2021). Potent and protective IGHV3-53/3-66 public antibodies and their shared escape mutant on the spike of SARS-CoV-2. Nature communications 12, 4210. 10.1038/s41467-021-24514-w.
Starr, T. N., Czudnochowski, N., Liu, Z., Zatta, F., Park, Y.-J., Addetia, A., Pinto, D., Beltramello, M., Hernandez, P., Greaney, A. J., et al. (2021). SARS-CoV-2 RBD antibodies that maximize breadth and resistance to escape. Nature 597, 97-102. 10.1038/s41586-021-03807-6.
Kramer, K. J., Johnson, N. V., Shiakolas, A. R., Suryadevara, N., Periasamy, S., Raju, N., Williams, J. K., Wrapp, D., Zost, S. J., Walker, L. M., et al. (2021). Potent neutralization of SARS-CoV-2 variants of concern by an antibody with an uncommon genetic signature and structural mode of spike recognition. Cell Rep 37, 109784. 10.1016/j.celrep.2021.109784.
Vlasova, A. N., Diaz, A., Damtie, D., Xiu, L., Toh, T. H., Lee, J. S., Saif, L. J., and Gray, G. C. (2022). Novel Canine Coronavirus Isolated from a Hospitalized Patient With Pneumonia in East Malaysia. Clin Infect Dis 74, 446-454. 10.1093/cid/ciab456.
Georgiev, I. S., Joyce, M. G., Yang, Y., Sastry, M., Zhang, B., Baxa, U., Chen, R. E., Druz, A., Lees, C. R., Narpala, S., et al. (2015). Single-Chain Soluble BG505.SOSIP gp140 Trimers as Structural and Antigenic Mimics of Mature Closed HIV-1 Env. Journal of Virology 89, 5318-5329. 10.1128/jvi.03451-14.
Alamyar, E., Duroux, P., Lefranc, M.-P., and Giudicelli, V. (2012). IMGT® Tools for the Nucleotide Analysis of Immunoglobulin (IG) and T Cell Receptor (TR) V-(D)-J Repertoires,
Polymorphisms, and IG Mutations: IMGT/V-QUEST and IMGT/HighV-QUEST for NGS. In (Humana Press), pp. 569-604. 10.1007/978-1-61779-842-9_32.
Gupta, N. T., Vander Heiden, J. A., Uduman, M., Gadala-Maria, D., Yaari, G., and Kleinstein, S. H. (2015). Change-O: a toolkit for analyzing large-scale B cell immunoglobulin repertoire sequencing data. Bioinformatics 31, 3356-3358. 10.1093/bioinformatics/btv359.
Crawford, K. H. D., Eguia, R., Dingens, A. S., Loes, A. N., Malone, K. D., Wolf, C. R., Chu, H. Y., Tortorici, M. A., Veesler, D., Murphy, M., et al. (2020). Protocol and Reagents for Pseudotyping Lentiviral Particles with SARS-CoV-2 Spike Protein for Neutralization Assays. Viruses 12, 513. 10.3390/v12050513.
Kaas, Q., Ruiz, M., and Lefranc, M. P. (2004). IMGT/3Dstructure-DB and IMGT/StructuralQuery, a database and a tool for immunoglobulin, T cell receptor and MHC structural data. Nucleic Acids Res 32, D208-210. 10.1093/nar/gkh042.
Ohi, M., Li, Y., Cheng, Y., and Walz, T. (2004). Negative staining and image classification—powerful tools in modern electron microscopy. Biological Procedures Online 6, 23-34. 10.1251/bpo70.
Mastronarde, D. N. (2003). SerialEM: A Program for Automated Tilt Series Acquisition on Tecnai Microscopes Using Prediction of Specimen Position. Microscopy and Microanalysis 9, 1182-1183. 10.1017/s1431927603445911.
Punjani, A., Rubinstein, J. L., Fleet, D. J., and Brubaker, M. A. (2017). cryoSPARC: algorithms for rapid unsupervised cryo-EM structure determination. Nature Methods 14, 290-296. 10.1038/nmeth.4169.
Pettersen, E. F., Goddard, T. D., Huang, C. C., Meng, E. C., Couch, G. S., Croll, T. I., Morris, J. H., and Ferrin, T. E. (2021). <scp>UCSF ChimeraX</scp>: Structure visualization for researchers, educators, and developers. Protein Science 30, 70-82. 10.1002/pro.3943.
Copin, R., Baum, A., Wloga, E., Pascal, K. E., Giordano, S., Fulton, B. O., Zhou, A., Negron, N., Lanza, K., Chan, N., et al. (2021). The monoclonal antibody combination REGEN-COV protects against SARS-CoV-2 mutational escape in preclinical and human studies. Cell 184, 3949-3961 e3911. 10.1016/j.cell.2021.06.002.

TABLE 2

| | | | SEQ ID | | | |
|---|---|---|---|---|---|---|
| | SEQ ID | | NO of | | | |
| | NO of | | SEQUENCE_{—} | | IN_{—} | |
| Name | Barcode | CONTIG.H | INPUT.H | FUNCTIONAL.H | FRAME.H | INDELS.H |
| 71281-1 | 1 | contig_2 | 60 | T | T | F |
| 71281-2 | 2 | contig_2 | 61 | T | T | T |
| 71281-3 | 3 | contig_1 | 62 | T | T | F |
| 71281-4 | 4 | contig_1 | 63 | T | T | T |
| 71281-5 | 5 | contig_2 | 64 | T | T | F |
| 71281-6 | 6 | contig_2 | 65 | T | T | F |
| 71281-7 | 7 | contig_1 | 66 | T | T | F |
| 71281-8 | 8 | contig_2 | 67 | T | T | F |
| 71281-9 | 9 | contig_2 | 68 | T | T | F |
| 71281-10 | 10 | contig_2 | 69 | T | T | F |
| 71281-11 | 11 | contig_2 | 70 | T | T | F |
| 71281-12 | 12 | contig_2 | 71 | T | T | F |
| 71281-13 | 13 | contig_1 | 72 | T | T | F |
| 71281-14 | 14 | contig_2 | 73 | T | T | F |
| 71281-15 | 15 | contig_1 | 74 | T | T | F |
| 71281-16 | 16 | contig_2 | 75 | T | T | F |
| 71281-17 | 17 | contig_2 | 76 | T | T | F |
| 71281-18 | 18 | contig_2 | 77 | T | T | F |
| 71281-19 | 19 | contig_1 | 78 | T | T | F |
| 71281-20 | 20 | contig_2 | 79 | T | T | F |
| 71281-21 | 21 | contig_1 | 80 | T | T | T |
| 71281-22 | 22 | contig_2 | 81 | T | T | F |
| 71281-23 | 23 | contig_2 | 82 | T | T | F |
| 71281-24 | 24 | contig_2 | 83 | T | T | F |
| 71281-25 | 25 | contig_1 | 84 | T | T | F |
| 71281-26 | 26 | contig_1 | 85 | T | T | F |
| 71281-27 | 27 | contig_2 | 86 | T | T | F |

| | | | | | | SEQ ID |
|---|---|---|---|---|---|---|
| | | | | | | NO of |
| | | | | | | HC |
| | | | | | SEQ ID | seq |
| | | V_{—} | D_{—} | J_{—} | NO of | VDJ |
| Name | LOCUS.H | CALL.H | CALL.H | CALL.H | VDJ.H | aa |
| 71281-1 | IGH | IGHV3-23 | IGHD3-9 | IGHJ5 | 119 | 178 |
| 71281-2 | IGH | IGHV1-46 | IGHD2-21 | IGHJ4 | 120 | 179 |
| 71281-3 | IGH | IGHV3-7 | IGHD2-2 | IGHJ4 | 121 | 180 |
| 71281-4 | IGH | IGHV3-64D | IGHD3-10 | IGHJ4 | 122 | 181 |
| 71281-5 | IGH | IGHV3-23 | IGHD3-3 | IGHJ4 | 123 | 182 |
| 71281-6 | IGH | IGHV3-30 | IGHD4-17 | IGHJ4 | 124 | 183 |
| 71281-7 | IGH | IGHV3-23 | IGHD6-13 | IGHJ4 | 125 | 184 |
| 71281-8 | IGH | IGHV4-39 | IGHD6-13 | IGHJ4 | 126 | 185 |
| 71281-9 | IGH | IGHV5-51 | IGHD3-16 | IGHJ3 | 127 | 186 |
| 71281-10 | IGH | IGHV3-7 | IGHD4-17 | IGHJ4 | 128 | 187 |
| 71281-11 | IGH | IGHV1-69 | IGHD6-6 | IGHJ4 | 129 | 188 |
| 71281-12 | IGH | IGHV4-39 | IGHD6-13 | IGHJ4 | 130 | 189 |
| 71281-13 | IGH | IGHV1-18 | IGHD2-15 | IGHJ4 | 131 | 190 |
| 71281-14 | IGH | IGHV3-11 | IGHD2-21 | IGHJ4 | 132 | 191 |
| 71281-15 | IGH | IGHV3-15 | IGHD3-10 | IGHJ6 | 133 | 192 |
| 71281-16 | IGH | IGHV3-73 | IGHD6-6 | IGHJ4 | 134 | 193 |
| 71281-17 | IGH | IGHV3-74 | IGHD3-10 | IGHJ3 | 135 | 194 |
| 71281-18 | IGH | IGHV3-53 | IGHD5-24 | IGHJ1 | 136 | 195 |
| 71281-19 | IGH | IGHV1-2 | IGHD3-22 | IGHJ6 | 137 | 196 |
| 71281-20 | IGH | IGHV3-74 | IGHD3-10 | IGHJ5 | 138 | 197 |
| 71281-21 | IGH | IGHV4-39 | IGHD6-6 | IGHJ6 | 139 | 198 |
| 71281-22 | IGH | IGHV4-31 | IGHD4-17 | IGHJ6 | 140 | 199 |
| 71281-23 | IGH | IGHV4-4 | IGHD6-13 | IGHJ4 | 141 | 200 |
| 71281-24 | IGH | IGHV5-10-1 | IGHD4-23 | IGHJ3 | 142 | 201 |
| 71281-25 | IGH | IGHV3-23 | IGHD2-2 | IGHJ1 | 143 | 202 |
| 71281-26 | IGH | IGHV3-23 | IGHD4-17 | IGHJ2 | 144 | 203 |
| 71281-27 | IGH | IGHV4-39 | IGHD6-19 | IGHJ3 | 145 | 204 |

TABLE 3

| | | | | | | | | | SEQ ID |
|---|---|---|---|---|---|---|---|---|---|
| | SEQ ID | SEQ ID | | | | | | | NO of |
| | NO of | NO of | JUNCTION_{—} | GERMLINE_{—} | V_{—} | V_{—} | J_{—} | J_{—} | FWR1_{—} |
| Name | IMGT.H | JUNCTION.H | LENGTH.H | IMGT.H | SCORE.H | IDENTITY.H | SCORE.H | IDENTITY.H | IMGT.H |
| 71281- | 237 | 296 | 66 | NA | 1350 | 0.9653 | 246 | 0.9804 | 355 |
| 1 | | | | | | | | | |
| 71281- | 238 | 297 | 51 | NA | 1035 | 0.8438 | 168 | 0.8333 | 356 |
| 2 | | | | | | | | | |
| 71281- | 239 | 298 | 42 | NA | 1359 | 0.9688 | 204 | 0.9167 | 357 |
| 3 | | | | | | | | | |
| 71281- | 240 | 299 | 78 | NA | 1359 | 0.9688 | 231 | 0.9792 | 358 |
| 4 | | | | | | | | | |
| 71281- | 241 | 300 | 60 | NA | 1332 | 0.9583 | 186 | 0.875 | 359 |
| 5 | | | | | | | | | |
| 71281- | 242 | 301 | 48 | NA | 1305 | 0.9479 | 186 | 0.875 | 360 |
| 6 | | | | | | | | | |
| 71281- | 243 | 302 | 42 | NA | 1170 | 0.8958 | 231 | 0.9792 | 361 |
| 7 | | | | | | | | | |
| 71281- | 244 | 303 | 54 | NA | 1329 | 0.9519 | 186 | 0.875 | 362 |
| 8 | | | | | | | | | |
| 71281- | 245 | 304 | 48 | NA | 1332 | 0.9583 | 169 | 0.82 | 363 |
| 9 | | | | | | | | | |
| 71281- | 246 | 305 | 36 | NA | 1215 | 0.9132 | 213 | 0.9375 | 364 |
| 10 | | | | | | | | | |
| 71281- | 247 | 306 | 45 | NA | 1188 | 0.9028 | 168 | 0.8333 | 365 |
| 11 | | | | | | | | | |
| 71281- | 248 | 307 | 66 | NA | 1392 | 0.9759 | 195 | 0.8958 | 366 |
| 12 | | | | | | | | | |
| 71281- | 249 | 308 | 63 | NA | 1251 | 0.9271 | 177 | 0.8542 | 367 |
| 13 | | | | | | | | | |
| 71281- | 250 | 309 | 60 | NA | 1350 | 0.9653 | 204 | 0.9167 | 368 |
| 14 | | | | | | | | | |
| 71281- | 251 | 310 | 63 | NA | 1209 | 0.9014 | 220 | 0.8387 | 369 |
| 15 | | | | | | | | | |
| 71281- | 252 | 311 | 39 | NA | 1452 | 0.9932 | 213 | 0.9375 | 370 |
| 16 | | | | | | | | | |
| 71281- | 253 | 312 | 60 | NA | 1341 | 0.9618 | 142 | 0.76 | 371 |
| 17 | | | | | | | | | |
| 71281- | 254 | 313 | 27 | NA | 1182 | 0.9053 | 128 | 0.7347 | 372 |
| 18 | | | | | | | | | |
| 71281- | 255 | 314 | 75 | NA | 1422 | 0.9931 | 256 | 0.9032 | 373 |
| 19 | | | | | | | | | |
| 71281- | 256 | 315 | 51 | NA | 1269 | 0.934 | 228 | 0.9412 | 374 |
| 20 | | | | | | | | | |
| 71281- | 257 | 316 | 57 | NA | 1338 | 0.9553 | 256 | 0.9032 | 375 |
| 21 | | | | | | | | | |
| 71281- | 258 | 317 | 54 | NA | 1365 | 0.9656 | 256 | 0.9032 | 376 |
| 22 | | | | | | | | | |
| 71281- | 259 | 318 | 51 | NA | 1278 | 0.9375 | 204 | 0.9167 | 377 |
| 23 | | | | | | | | | |
| 71281- | 260 | 319 | 42 | NA | 1413 | 0.9896 | 223 | 0.94 | 378 |
| 24 | | | | | | | | | |
| 71281- | 261 | 320 | 60 | NA | 1386 | 0.9792 | 152 | 0.7692 | 379 |
| 25 | | | | | | | | | |
| 71281- | 262 | 321 | 48 | NA | 1287 | 0.941 | 238 | 0.9434 | 380 |
| 26 | | | | | | | | | |
| 71281- | 263 | 322 | 60 | NA | 1356 | 0.9622 | 214 | 0.92 | 381 |
| 27 | | | | | | | | | |

TABLE 4

| | SEQ ID | SEQ ID | SEQ ID | SEQ ID | SEQ ID | SEQ ID | | | |
|---|---|---|---|---|---|---|---|---|---|
| | NO of | NO of | NO of | NO of | NO of | NO of | | | |
| | FWR2_{—} | FWR3_{—} | FWR4_{—} | CDR1_{—} | CDR2_{—} | CDR3_{—} | C_{—} | | |
| Name | IMGT.H | IMGT.H | IMGT.H | IMGT.H | IMGT.H | IMGT.H | CALL.H | CONSCOUNT.H | UMICOUNT.H |
| 71281- | 414 | 473 | 532 | 591 | 650 | 709 | IGHG1 | 3278 | 4 |
| 1 | | | | | | | | | |
| 71281- | 415 | 474 | 533 | 592 | 651 | 710 | IGHG1 | 2826 | 5 |
| 2 | | | | | | | | | |
| 71281- | 416 | 475 | 534 | 593 | 652 | 711 | IGHG1 | 950 | 3 |
| 3 | | | | | | | | | |
| 71281- | 417 | 476 | 535 | 594 | 653 | 712 | IGHG2 | 538 | 2 |
| 4 | | | | | | | | | |
| 71281- | 418 | 477 | 536 | 595 | 654 | 713 | IGHG1 | 1568 | 4 |
| 5 | | | | | | | | | |
| 71281- | 419 | 478 | 537 | 596 | 655 | 714 | IGHA1 | 32216 | 29 |
| 6 | | | | | | | | | |
| 71281- | 420 | 479 | 538 | 597 | 656 | 715 | IGHG1 | 11106 | 7 |
| 7 | | | | | | | | | |
| 71281- | 421 | 480 | 539 | 598 | 657 | 716 | IGHG1 | 2860 | 4 |
| 8 | | | | | | | | | |
| 71281- | 422 | 481 | 540 | 599 | 658 | 717 | IGHG1 | 9778 | 7 |
| 9 | | | | | | | | | |
| 71281- | 423 | 482 | 541 | 600 | 659 | 718 | IGHG1 | 986 | 3 |
| 10 | | | | | | | | | |
| 71281- | 424 | 483 | 542 | 601 | 660 | 719 | IGHG1 | 14506 | 9 |
| 11 | | | | | | | | | |
| 71281- | 425 | 484 | 543 | 602 | 661 | 720 | IGHG1 | 12660 | 16 |
| 12 | | | | | | | | | |
| 71281- | 426 | 485 | 544 | 603 | 662 | 721 | IGHA1 | 45922 | 38 |
| 13 | | | | | | | | | |
| 71281- | 427 | 486 | 545 | 604 | 663 | 722 | IGHA1 | 7476 | 6 |
| 14 | | | | | | | | | |
| 71281- | 428 | 487 | 546 | 605 | 664 | 723 | IGHG1 | 4062 | 3 |
| 15 | | | | | | | | | |
| 71281- | 429 | 488 | 547 | 606 | 665 | 724 | IGHG3 | 180 | 1 |
| 16 | | | | | | | | | |
| 71281- | 430 | 489 | 548 | 607 | 666 | 725 | IGHG3 | 4632 | 8 |
| 17 | | | | | | | | | |
| 71281- | 431 | 490 | 549 | 608 | 667 | 726 | IGHG1 | 878 | 2 |
| 18 | | | | | | | | | |
| 71281- | 432 | 491 | 550 | 609 | 668 | 727 | IGHA1 | 26226 | 23 |
| 19 | | | | | | | | | |
| 71281- | 433 | 492 | 551 | 610 | 669 | 728 | IGHG1 | 3096 | 8 |
| 20 | | | | | | | | | |
| 71281- | 434 | 493 | 552 | 611 | 670 | 729 | IGHG1 | 6158 | 10 |
| 21 | | | | | | | | | |
| 71281- | 435 | 494 | 553 | 612 | 671 | 730 | IGHG1 | 3146 | 6 |
| 22 | | | | | | | | | |
| 71281- | 436 | 495 | 554 | 613 | 672 | 731 | IGHG2 | 2948 | 3 |
| 23 | | | | | | | | | |
| 71281- | 437 | 496 | 555 | 614 | 673 | 732 | IGHG1 | 9386 | 8 |
| 24 | | | | | | | | | |
| 71281- | 438 | 497 | 556 | 615 | 674 | 733 | IGHA1 | 4308 | 5 |
| 25 | | | | | | | | | |
| 71281- | 439 | 498 | 557 | 616 | 675 | 734 | IGHA1 | 28754 | 26 |
| 26 | | | | | | | | | |
| 71281- | 440 | 499 | 558 | 617 | 676 | 735 | IGHA1 | 28460 | 17 |
| 27 | | | | | | | | | |

TABLE 5

| | SEQ ID | SEQ ID | | | SEQ ID | | | | |
|---|---|---|---|---|---|---|---|---|---|
| | NO of | NO of | | | NO of | | | | |
| | JUNCTION_{—} | CDRH3 | | | SEQUENCE_{—} | | IN_{—} | | |
| Name | 10X_AA.H | aa seq | CLONE | CONTIG.L | INPUT.L | FUNCTIONAL.L | FRAME.L | INDELS.L | LOCUS.L |
| 71281- | 768 | 827 | 51 | contig_1 | 886 | T | T | F | IGL |
| 1 | | | | | | | | | |
| 71281- | 769 | 828 | 132 | contig_1 | 887 | T | T | F | IGK |
| 2 | | | | | | | | | |
| 71281- | 770 | 829 | 550 | contig_2 | 888 | T | T | F | IGL |
| 3 | | | | | | | | | |
| 71281- | 771 | 830 | 810 | contig_2 | 889 | T | T | F | IGK |
| 4 | | | | | | | | | |
| 71281- | 772 | 831 | 1599 | contig_1 | 890 | T | T | F | IGL |
| 5 | | | | | | | | | |
| 71281- | 773 | 832 | 2002 | contig_1 | 891 | T | T | F | IGK |
| 6 | | | | | | | | | |
| 71281- | 774 | 833 | 2091 | contig_2 | 892 | T | T | F | IGL |
| 7 | | | | | | | | | |
| 71281- | 775 | 834 | 2128 | contig_1 | 893 | T | T | F | IGK |
| 8 | | | | | | | | | |
| 71281- | 776 | 835 | 2408 | contig_1 | 894 | T | T | F | IGL |
| 9 | | | | | | | | | |
| 71281- | 777 | 836 | 2417 | contig_1 | 895 | T | T | F | IGK |
| 10 | | | | | | | | | |
| 71281- | 778 | 837 | 2549 | contig_1 | 896 | T | T | T | IGK |
| 11 | | | | | | | | | |
| 71281- | 779 | 838 | 2622 | contig_1 | 897 | T | T | F | IGL |
| 12 | | | | | | | | | |
| 71281- | 780 | 839 | 2697 | contig_2 | 898 | T | T | F | IGK |
| 13 | | | | | | | | | |
| 71281- | 781 | 840 | 2739 | contig_1 | 899 | T | T | F | IGK |
| 14 | | | | | | | | | |
| 71281- | 782 | 841 | 2749 | contig_2 | 900 | T | T | F | IGK |
| 15 | | | | | | | | | |
| 71281- | 783 | 842 | 2849 | contig_1 | 901 | T | T | F | IGK |
| 16 | | | | | | | | | |
| 71281- | 784 | 843 | 3173 | contig_1 | 902 | T | T | F | IGK |
| 17 | | | | | | | | | |
| 71281- | 785 | 844 | 3298 | contig_1 | 903 | T | T | F | IGK |
| 18 | | | | | | | | | |
| 71281- | 786 | 845 | 3727 | contig_2 | 904 | T | T | F | IGL |
| 19 | | | | | | | | | |
| 71281- | 787 | 846 | 3881 | contig_1 | 905 | T | T | F | IGK |
| 20 | | | | | | | | | |
| 71281- | 788 | 847 | 4001 | contig_2 | 906 | T | T | F | IGK |
| 21 | | | | | | | | | |
| 71281- | 789 | 848 | 4012 | contig_1 | 907 | T | T | F | IGL |
| 22 | | | | | | | | | |
| 71281- | 790 | 849 | 4080 | contig_1 | 908 | T | T | F | IGK |
| 23 | | | | | | | | | |
| 71281- | 791 | 850 | 4108 | contig_1 | 909 | T | T | F | IGK |
| 24 | | | | | | | | | |
| 71281- | 792 | 851 | 4161 | contig_2 | 910 | T | T | F | IGK |
| 25 | | | | | | | | | |
| 71281- | 793 | 852 | 4187 | contig_2 | 911 | T | T | F | IGL |
| 26 | | | | | | | | | |
| 71281- | 794 | 853 | 4236 | contig_1 | 912 | T | T | F | IGL |
| 27 | | | | | | | | | |

TABLE 6

| | | | SEQ ID | SEQ ID | SEQ ID | | | | |
|---|---|---|---|---|---|---|---|---|---|
| | | | NO of | NO of | NO of | SEQ ID | | | |
| | V_{—} | J_{—} | SEQUENCE_{—} | LC seq | SEQUENCE_{—} | NO of | JUNCTION_{—} | GERMLINE_{—} | V_{—} |
| Name | CALL.L | CALL.L | VJ.L | VJ aa | IMGT.L | JUNCTION.L | LENGTH.L | IMGT.L | SCORE.L |
| 71281- | IGLV1- | IGLJ1 | 945 | 1004 | 1063 | 1122 | 39 | NA | 1398 |
| 1 | 51 | | | | | | | | |
| 71281- | IGKV4- | IGKJ4 | 946 | 1005 | 1064 | 1123 | 33 | NA | 1359 |
| 2 | 1 | | | | | | | | |
| 71281- | IGLV3- | IGLJ2 | 947 | 1006 | 1065 | 1124 | 33 | NA | 1278 |
| 3 | 1 | | | | | | | | |
| 71281- | IGKV3- | IGKJ2 | 948 | 1007 | 1066 | 1125 | 36 | NA | 1377 |
| 4 | 15 | | | | | | | | |
| 71281- | IGLV1- | IGLJ2 | 949 | 1008 | 1067 | 1126 | 39 | NA | 1353 |
| 5 | 51 | | | | | | | | |
| 71281- | IGKV1- | IGKJ3 | 950 | 1009 | 1068 | 1127 | 33 | NA | 1323 |
| 6 | 33 | | | | | | | | |
| 71281- | IGLV3- | IGLJ2 | 951 | 1010 | 1069 | 1128 | 39 | NA | 1260 |
| 7 | 25 | | | | | | | | |
| 71281- | IGKV1- | IGKJ4 | 952 | 1011 | 1070 | 1129 | 36 | NA | 1341 |
| 8 | 39 | | | | | | | | |
| 71281- | IGLV3- | IGLJ3 | 953 | 1012 | 1071 | 1130 | 39 | NA | 1377 |
| 9 | 10 | | | | | | | | |
| 71281- | IGKV2- | IGKJ1 | 954 | 1013 | 1072 | 1131 | 33 | NA | 1353 |
| 10 | 28 | | | | | | | | |
| 71281- | IGKV3- | IGKJ1 | 955 | 1014 | 1073 | 1132 | 33 | NA | 1314 |
| 11 | 20 | | | | | | | | |
| 71281- | IGLV3- | IGLJ3 | 956 | 1015 | 1074 | 1133 | 39 | NA | 1323 |
| 12 | 19 | | | | | | | | |
| 71281- | IGKV1- | IGKJ5 | 957 | 1016 | 1075 | 1134 | 36 | NA | 1242 |
| 13 | 33 | | | | | | | | |
| 71281- | IGKV3- | IGKJ5 | 958 | 1017 | 1076 | 1135 | 33 | NA | 1332 |
| 14 | 11 | | | | | | | | |
| 71281- | IGKV1- | IGKJ5 | 959 | 1018 | 1077 | 1136 | 36 | NA | 1251 |
| 15 | 9 | | | | | | | | |
| 71281- | IGKV3- | IGKJ1 | 960 | 1019 | 1078 | 1137 | 36 | NA | 1383 |
| 16 | 20 | | | | | | | | |
| 71281- | IGKV3- | IGKJ2 | 961 | 1020 | 1079 | 1138 | 33 | NA | 1323 |
| 17 | 11 | | | | | | | | |
| 71281- | IGKV2- | IGKJ1 | 962 | 1021 | 1080 | 1139 | 30 | NA | 1290 |
| 18 | 30 | | | | | | | | |
| 71281- | IGLV1- | IGLJ2 | 963 | 1022 | 1081 | 1140 | 39 | NA | 1398 |
| 19 | 47 | | | | | | | | |
| 71281- | IGKV4- | IGKJ1 | 964 | 1023 | 1082 | 1141 | 30 | NA | 1440 |
| 20 | 1 | | | | | | | | |
| 71281- | IGKV3- | IGKJ5 | 965 | 1024 | 1083 | 1142 | 36 | NA | 1341 |
| 21 | 11 | | | | | | | | |
| 71281- | IGLV1- | IGLJ1 | 966 | 1025 | 1084 | 1143 | 42 | NA | 1380 |
| 22 | 44 | | | | | | | | |
| 71281- | IGKV3- | IGKJ1 | 967 | 1026 | 1085 | 1144 | 33 | NA | 1302 |
| 23 | 20 | | | | | | | | |
| 71281- | IGKV4- | IGKJ1 | 968 | 1027 | 1086 | 1145 | 33 | NA | 1485 |
| 24 | 1 | | | | | | | | |
| 71281- | IGKV2- | IGKJ2 | 969 | 1028 | 1087 | 1146 | 33 | NA | 1416 |
| 25 | 30 | | | | | | | | |
| 71281- | IGLV2- | IGLJ3 | 970 | 1029 | 1088 | 1147 | 36 | NA | 1287 |
| 26 | 14 | | | | | | | | |
| 71281- | IGLV1- | IGLJ3 | 971 | 1030 | 1089 | 1148 | 39 | NA | 1371 |
| 27 | 51 | | | | | | | | |

TABLE 7

| | | | | SEQ ID | SEQ ID | SEQ ID | SEQ ID | SEQ ID | SEQ ID |
|---|---|---|---|---|---|---|---|---|---|
| | | | | NO of | NO of | NO of | NO of | NO of | NO of |
| | V_{—} | J_{—} | J_{—} | FWR1_{—} | FWR2_{—} | FWR3_{—} | FWR4_{—} | CDR1_{—} | CDR2_{—} |
| Name | IDENTITY.L | SCORE.L | IDENTITY.L | IMGT.L | IMGT.L | IMGT.LL | IMGT.LL | IMGT.L | IMGT.L |
| 71281-1 | 0.9895 | 167 | 0.9459 | 1181 | 1240 | 1299 | 1358 | 1417 | 1476 |
| 71281-2 | 0.9529 | 172 | 0.9474 | 1182 | 1241 | 1300 | 1359 | 1418 | 1477 |
| 71281-3 | 0.9534 | 157 | 0.9429 | 1183 | 1242 | 1301 | 1360 | 1419 | 1478 |
| 71281-4 | 0.9928 | 177 | 0.9487 | 1184 | 1243 | 1302 | 1361 | 1420 | 1479 |
| 71281-5 | 0.9719 | 180 | 1 | 1185 | 1244 | 1303 | 1362 | 1421 | 1480 |
| 71281-6 | 0.9713 | 181 | 0.9737 | 1186 | 1245 | 1304 | 1363 | 1422 | 1481 |
| 71281-7 | 0.9462 | 172 | 0.9474 | 1187 | 1246 | 1305 | 1364 | 1423 | 1482 |
| 71281-8 | 0.9785 | 172 | 0.9474 | 1188 | 1247 | 1306 | 1365 | 1424 | 1483 |
| 71281-9 | 0.9928 | 166 | 0.9714 | 1189 | 1248 | 1307 | 1366 | 1425 | 1484 |
| 71281- | 0.9558 | 181 | 0.9737 | 1190 | 1249 | 1308 | 1367 | 1426 | 1485 |
| 10 | | | | | | | | | |
| 71281- | 0.9677 | 171 | 0.9722 | 1191 | 1250 | 1309 | 1368 | 1427 | 1486 |
| 11 | | | | | | | | | |
| 71281- | 0.9713 | 176 | 0.973 | 1192 | 1251 | 1310 | 1369 | 1428 | 1487 |
| 12 | | | | | | | | | |
| 71281- | 0.9391 | 181 | 0.9737 | 1193 | 1252 | 1311 | 1370 | 1429 | 1488 |
| 13 | | | | | | | | | |
| 71281- | 0.9749 | 181 | 0.9737 | 1194 | 1253 | 1312 | 1371 | 1430 | 1489 |
| 14 | | | | | | | | | |
| 71281- | 0.9427 | 181 | 0.9737 | 1195 | 1254 | 1313 | 1372 | 1431 | 1490 |
| 15 | | | | | | | | | |
| 71281- | 0.9894 | 181 | 0.9737 | 1196 | 1255 | 1314 | 1373 | 1432 | 1491 |
| 16 | | | | | | | | | |
| 71281- | 0.9713 | 190 | 1 | 1197 | 1256 | 1315 | 1374 | 1433 | 1492 |
| 17 | | | | | | | | | |
| 71281- | 0.932 | 167 | 0.9459 | 1198 | 1257 | 1316 | 1375 | 1434 | 1493 |
| 18 | | | | | | | | | |
| 71281- | 0.9895 | 166 | 0.9714 | 1199 | 1258 | 1317 | 1376 | 1435 | 1494 |
| 19 | | | | | | | | | |
| 71281- | 0.9832 | 148 | 0.9143 | 1200 | 1259 | 1318 | 1377 | 1436 | 1495 |
| 20 | | | | | | | | | |
| 71281- | 0.9785 | 181 | 0.9737 | 1201 | 1260 | 1319 | 1378 | 1437 | 1496 |
| 21 | | | | | | | | | |
| 71281- | 0.9825 | 190 | 1 | 1202 | 1261 | 1320 | 1379 | 1438 | 1497 |
| 22 | | | | | | | | | |
| 71281- | 0.9574 | 181 | 0.9737 | 1203 | 1262 | 1321 | 1380 | 1439 | 1498 |
| 23 | | | | | | | | | |
| 71281- | 1 | 190 | 1 | 1204 | 1263 | 1322 | 1381 | 1440 | 1499 |
| 24 | | | | | | | | | |
| 71281- | 0.9796 | 181 | 0.9737 | 1205 | 1264 | 1323 | 1382 | 1441 | 1500 |
| 25 | | | | | | | | | |
| 71281- | 0.941 | 185 | 1 | 1206 | 1265 | 1324 | 1383 | 1442 | 1501 |
| 26 | | | | | | | | | |
| 71281- | 0.9789 | 162 | 0.9444 | 1207 | 1266 | 1325 | 1384 | 1443 | 1502 |
| 27 | | | | | | | | | |

TABLE 8

| | SEQ ID | | | SEQ ID | | | | | | |
|---|---|---|---|---|---|---|---|---|---|---|
| | NO of | | | NO of | SEQ ID | SEQ ID | SEQ ID | SEQ ID | SEQ ID | SEQ ID |
| | CDR3_{—} | C_{—} | | JUNCTION_{—} | NO of | NO of | NO of | NO of | NO of | NO of |
| Name | IMGT.L | CALL.L | CONSCOUNT.L | 10X_AA.L | CDRH1 | CDRH2 | CDRH3 | CDRL1 | CDRL2 | CDRL3 |
| 71281-1 | 1535 | IGLC1 | 18120 | 1594 | 1653 | 1712 | 1771 | 1830 | 1889 | 1948 |
| 71281-2 | 1536 | IGKC | 62466 | 1595 | 1654 | 1713 | 1772 | 1831 | 1890 | 1949 |
| 71281-3 | 1537 | IGLC2 | 20046 | 1596 | 1655 | 1714 | 1773 | 1832 | 1891 | 1950 |
| 71281-4 | 1538 | IGKC | 53330 | 1597 | 1656 | 1715 | 1774 | 1833 | 1892 | 1951 |
| 71281-5 | 1539 | IGLC2 | 18364 | 1598 | 1657 | 1716 | 1775 | 1834 | 1893 | 1952 |
| 71281-6 | 1540 | IGKC | 43508 | 1599 | 1658 | 1717 | 1776 | 1835 | 1894 | 1953 |
| 71281-7 | 1541 | IGLC2 | 11934 | 1600 | 1659 | 1718 | 1777 | 1836 | 1895 | 1954 |
| 71281-8 | 1542 | IGKC | 5936 | 1601 | 1660 | 1719 | 1778 | 1837 | 1896 | 1955 |
| 71281-9 | 1543 | IGLC2 | 6426 | 1602 | 1661 | 1720 | 1779 | 1838 | 1897 | 1956 |
| 71281-10 | 1544 | IGKC | 50438 | 1603 | 1662 | 1721 | 1780 | 1839 | 1898 | 1957 |
| 71281-11 | 1545 | IGKC | 19104 | 1604 | 1663 | 1722 | 1781 | 1840 | 1899 | 1958 |
| 71281-12 | 1546 | IGLC3 | 61708 | 1605 | 1664 | 1723 | 1782 | 1841 | 1900 | 1959 |
| 71281-13 | 1547 | IGKC | 23620 | 1606 | 1665 | 1724 | 1783 | 1842 | 1901 | 1960 |
| 71281-14 | 1548 | IGKC | 5640 | 1607 | 1666 | 1725 | 1784 | 1843 | 1902 | 1961 |
| 71281-15 | 1549 | IGKC | 6842 | 1608 | 1667 | 1726 | 1785 | 1844 | 1903 | 1962 |
| 71281-16 | 1550 | IGKC | 12900 | 1609 | 1668 | 1727 | 1786 | 1845 | 1904 | 1963 |
| 71281-17 | 1551 | IGKC | 41334 | 1610 | 1669 | 1728 | 1787 | 1846 | 1905 | 1964 |
| 71281-18 | 1552 | IGKC | 24216 | 1611 | 1670 | 1729 | 1788 | 1847 | 1906 | 1965 |
| 71281-19 | 1553 | IGLC2 | 12306 | 1612 | 1671 | 1730 | 1789 | 1848 | 1907 | 1966 |
| 71281-20 | 1554 | IGKC | 10740 | 1613 | 1672 | 1731 | 1790 | 1849 | 1908 | 1967 |
| 71281-21 | 1555 | IGKC | 70580 | 1614 | 1673 | 1732 | 1791 | 1850 | 1909 | 1968 |
| 71281-22 | 1556 | IGLC1 | 28500 | 1615 | 1674 | 1733 | 1792 | 1851 | 1910 | 1969 |
| 71281-23 | 1557 | IGKC | 38128 | 1616 | 1675 | 1734 | 1793 | 1852 | 1911 | 1970 |
| 71281-24 | 1558 | IGKC | 17222 | 1617 | 1676 | 1735 | 1794 | 1853 | 1912 | 1971 |
| 71281-25 | 1559 | IGKC | 11374 | 1618 | 1677 | 1736 | 1795 | 1854 | 1913 | 1972 |
| 71281-26 | 1560 | IGLC2 | 42902 | 1619 | 1678 | 1737 | 1796 | 1855 | 1914 | 1973 |
| 71281-27 | 1561 | IGLC2 | 22530 | 1620 | 1679 | 1738 | 1797 | 1856 | 1915 | 1974 |

TABLE 9

| | | | SEQ ID | | | |
|---|---|---|---|---|---|---|
| | SEQ ID | | NO of | | | |
| | NO of | | SEQUENCE_{—} | | IN_{—} | |
| Name | Barcode | CONTIG.H | INPUT.H | FUNCTIONAL.H | FRAME.H | INDELS.H |
| 71281- | 28 | contig_2 | 87 | T | T | F |
| 28 | | | | | | |
| 71281- | 29 | contig_2 | 88 | T | T | F |
| 29 | | | | | | |
| 71281- | 30 | contig_2 | 89 | T | T | T |
| 30 | | | | | | |
| 71281- | 31 | contig_2 | 90 | T | T | F |
| 31 | | | | | | |
| 71281- | 32 | contig_2 | 91 | T | T | F |
| 32 | | | | | | |
| 71281- | 33 | contig_2 | 92 | T | T | F |
| 33 | | | | | | |
| 71281- | 34 | contig_2 | 93 | T | T | T |
| 34 | | | | | | |
| 71281- | 35 | contig_2 | 94 | T | T | F |
| 35 | | | | | | |
| 71281- | 36 | contig_1 | 95 | T | T | F |
| 36 | | | | | | |
| 71281- | 37 | contig_2 | 96 | T | T | F |
| 37 | | | | | | |
| 71281- | 38 | contig_2 | 97 | T | T | F |
| 38 | | | | | | |
| 71281- | 39 | contig_2 | 98 | T | T | F |
| 39 | | | | | | |
| 71282- | 40 | contig_2 | 99 | T | T | F |
| 1 | | | | | | |
| 71282- | 41 | contig_2 | 100 | T | T | T |
| 2 | | | | | | |
| 71282- | 42 | contig_2 | 101 | T | T | T |
| 3 | | | | | | |
| 71282- | 43 | contig_1 | 102 | T | T | F |
| 4 | | | | | | |
| 71282- | 44 | contig_2 | 103 | T | T | F |
| 5 | | | | | | |
| 71282- | 45 | contig_2 | 104 | T | T | F |
| 6 | | | | | | |
| 71282- | 46 | contig_2 | 105 | T | T | F |
| 7 | | | | | | |
| 71282- | 47 | contig_2 | 106 | T | T | F |
| 8 | | | | | | |
| 71282- | 48 | contig_2 | 107 | T | T | F |
| 9 | | | | | | |
| 71282- | 49 | contig_1 | 108 | T | T | F |
| 10 | | | | | | |
| 71282- | 50 | contig_2 | 109 | T | T | F |
| 11 | | | | | | |
| 71282- | 51 | contig_2 | 110 | T | T | F |
| 12 | | | | | | |
| 71282- | 52 | contig_2 | 111 | T | T | F |
| 13 | | | | | | |
| 71282- | 53 | contig_2 | 112 | T | T | F |
| 14 | | | | | | |
| 71282- | 54 | contig_2 | 113 | T | T | F |
| 15 | | | | | | |
| 71282- | 55 | contig_1 | 114 | T | T | F |
| 16 | | | | | | |
| 71282- | 56 | contig_2 | 115 | T | T | F |
| 17 | | | | | | |
| 71282- | 57 | contig_2 | 116 | T | T | F |
| 18 | | | | | | |
| 71282- | 58 | contig_1 | 117 | T | T | F |
| 19 | | | | | | |
| 71282- | 59 | contig_1 | 118 | T | T | F |
| 20 | | | | | | |

| | | | | | | SEQ ID |
|---|---|---|---|---|---|---|
| | | | | | SEQ ID | NO of |
| | | V_{—} | D_{—} | J_{—} | NO of | HC seq |
| Name | LOCUS.H | CALL.H | CALL.H | CALL.H | VDJ.H | VDJ aa |
| 71281- | IGH | IGHV1- | IGHD3- | IGHJ6 | 146 | 205 |
| 28 | | 2 | 3 | | | |
| 71281- | IGH | IGHV5- | IGHD4- | IGHJ6 | 147 | 206 |
| 29 | | 10-1 | 17 | | | |
| 71281- | IGH | IGHV3- | IGHD1- | IGHJ4 | 148 | 207 |
| 30 | | 11 | 26 | | | |
| 71281- | IGH | IGHV3- | IGHD2- | IGHJ4 | 149 | 208 |
| 31 | | 30 | 15 | | | |
| 71281- | IGH | IGHV2- | IGHD4- | IGHJ4 | 150 | 209 |
| 32 | | 5 | 11 | | | |
| 71281- | IGH | IGHV3- | IGHD3- | IGHJ4 | 151 | 210 |
| 33 | | 66 | 16 | | | |
| 71281- | IGH | IGHV3- | IGHD6- | IGHJ4 | 152 | 211 |
| 34 | | 30-3 | 13 | | | |
| 71281- | IGH | IGHV3- | IGHD4- | IGHJ5 | 153 | 212 |
| 35 | | 53 | 17 | | | |
| 71281- | IGH | IGHV1- | IGHD2- | IGHJ3 | 154 | 213 |
| 36 | | 58 | 15 | | | |
| 71281- | IGH | IGHV3- | IGHD2- | IGHJ1 | 155 | 214 |
| 37 | | 53 | 15 | | | |
| 71281- | IGH | IGHV7- | IGHD2- | IGHJ6 | 156 | 215 |
| 38 | | 4-1 | 2 | | | |
| 71281- | IGH | IGHV3- | IGHD1- | IGHJ6 | 157 | 216 |
| 39 | | 30-3 | 26 | | | |
| 71282- | IGH | IGHV3- | IGHD3- | IGHJ4 | 158 | 217 |
| 1 | | 23 | 22 | | | |
| 71282- | IGH | IGHV3- | IGHD2- | IGHJ4 | 159 | 218 |
| 2 | | 23 | 2 | | | |
| 71282- | IGH | IGHV4- | IGHD5- | IGHJ4 | 160 | 219 |
| 3 | | 39 | 24 | | | |
| 71282- | IGH | IGHV1- | IGHD6- | IGHJ4 | 161 | 220 |
| 4 | | 69 | 19 | | | |
| 71282- | IGH | IGHV3- | IGHD4- | IGHJ4 | 162 | 221 |
| 5 | | 23 | 17 | | | |
| 71282- | IGH | IGHV3- | IGHD3- | IGHJ3 | 163 | 222 |
| 6 | | 48 | 22 | | | |
| 71282- | IGH | IGHV3- | IGHD1- | IGHJ5 | 164 | 223 |
| 7 | | 30-3 | 26 | | | |
| 71282- | IGH | IGHV3- | IGHD6- | IGHJ4 | 165 | 224 |
| 8 | | 7 | 13 | | | |
| 71282- | IGH | IGHV3- | IGHD1- | IGHJ4 | 166 | 225 |
| 9 | | 21 | 26 | | | |
| 71282- | IGH | IGHV3- | IGHD1- | IGHJ3 | 167 | 226 |
| 10 | | 30 | 26 | | | |
| 71282- | IGH | IGHV3- | IGHD1- | IGHJ6 | 168 | 227 |
| 11 | | 53 | 26 | | | |
| 71282- | IGH | IGHV1- | IGHD2- | IGHJ4 | 169 | 228 |
| 12 | | 69 | 2 | | | |
| 71282- | IGH | IGHV3- | IGHD1- | IGHJ3 | 170 | 229 |
| 13 | | 66 | 26 | | | |
| 71282- | IGH | IGHV3- | IGHD2- | IGHJ4 | 171 | 230 |
| 14 | | 30 | 15 | | | |
| 71282- | IGH | IGHV3- | IGHD3- | IGHJ4 | 172 | 231 |
| 15 | | 23 | 10 | | | |
| 71282- | IGH | IGHV1- | IGHD2- | IGHJ4 | 173 | 232 |
| 16 | | 3 | 15 | | | |
| 71282- | IGH | IGHV1- | IGHD1- | IGHJ4 | 174 | 233 |
| 17 | | 46 | 26 | | | |
| 71282- | IGH | IGHV4- | IGHD3- | IGHJ3 | 175 | 234 |
| 18 | | 59 | 22 | | | |
| 71282- | IGH | IGHV3- | IGHD2- | IGHJ2 | 176 | 235 |
| 19 | | 7 | 8 | | | |
| 71282- | IGH | IGHV2- | IGHD3- | IGHJ4 | 177 | 236 |
| 20 | | 5 | 22 | | | |

TABLE 10

| | | | | | | | | | SEQ ID |
|---|---|---|---|---|---|---|---|---|---|
| | SEQ ID | SEQ ID | | | | | | | NO of |
| | NO of | NO of | JUNCTION_{—} | GERMLINE_{—} | V_{—} | V_{—} | J_{—} | J_{—} | FWR1_{—} |
| Name | IMGT.H | JUNCTION.H | LENGTH.H | IMGT.H | SCORE.H | IDENTITY.H | SCORE.H | IDENTITY.H | IMGT.H |
| 71281- | 264 | 323 | 69 | NA | 1224 | 0.9167 | 238 | 0.871 | 382 |
| 28 | | | | | | | | | |
| 71281- | 265 | 324 | 57 | NA | 1395 | 0.9826 | 256 | 0.9032 | 383 |
| 29 | | | | | | | | | |
| 71281- | 266 | 325 | 48 | NA | 1134 | 0.8819 | 168 | 0.8333 | 384 |
| 30 | | | | | | | | | |
| 71281- | 267 | 326 | 69 | NA | 1359 | 0.9688 | 195 | 0.8958 | 385 |
| 31 | | | | | | | | | |
| 71281- | 268 | 327 | 39 | NA | 1401 | 0.9794 | 159 | 0.8125 | 386 |
| 32 | | | | | | | | | |
| 71281- | 269 | 328 | 39 | NA | 1281 | 0.9439 | 213 | 0.9375 | 387 |
| 33 | | | | | | | | | |
| 71281- | 270 | 329 | 51 | NA | 1170 | 0.8958 | 222 | 0.9583 | 388 |
| 34 | | | | | | | | | |
| 71281- | 27 | 330 | 39 | NA | 1380 | 0.9825 | 183 | 0.8431 | 389 |
| 35 | | | | | | | | | |
| 71281- | 272 | 331 | 54 | NA | 1305 | 0.9479 | 205 | 0.9 | 390 |
| 36 | | | | | | | | | |
| 71281- | 273 | 332 | 45 | NA | 1263 | 0.9368 | 170 | 0.8077 | 391 |
| 37 | | | | | | | | | |
| 71281- | 274 | 333 | 81 | NA | 1332 | 0.9583 | 265 | 0.9194 | 392 |
| 38 | | | | | | | | | |
| 71281- | 275 | 334 | 39 | NA | 1296 | 0.9444 | 218 | 0.8621 | 393 |
| 39 | | | | | | | | | |
| 71282- | 276 | 335 | 57 | NA | 1386 | 0.9792 | 222 | 0.9583 | 394 |
| 1 | | | | | | | | | |
| 71282- | 277 | 336 | 57 | NA | 1269 | 0.9498 | 204 | 0.9167 | 395 |
| 2 | | | | | | | | | |
| 71282- | 278 | 337 | 48 | NA | 1215 | 0.9132 | 186 | 0.875 | 396 |
| 3 | | | | | | | | | |
| 71282- | 279 | 338 | 51 | NA | 1359 | 0.9688 | 195 | 0.8958 | 397 |
| 4 | | | | | | | | | |
| 71282- | 280 | 339 | 54 | NA | 1314 | 0.9514 | 177 | 0.8542 | 398 |
| 5 | | | | | | | | | |
| 71282- | 281 | 340 | 63 | NA | 1314 | 0.9514 | 232 | 0.96 | 399 |
| 6 | | | | | | | | | |
| 71282- | 282 | 341 | 48 | NA | 1314 | 0.9514 | 237 | 0.9608 | 400 |
| 7 | | | | | | | | | |
| 71282- | 283 | 342 | 60 | NA | 1350 | 0.9653 | 222 | 0.9583 | 401 |
| 8 | | | | | | | | | |
| 71282- | 284 | 343 | 45 | NA | 1332 | 0.9583 | 159 | 0.8125 | 402 |
| 9 | | | | | | | | | |
| 71282- | 285 | 344 | 48 | NA | 1314 | 0.9514 | 214 | 0.92 | 403 |
| 10 | | | | | | | | | |
| 71282- | 286 | 345 | 51 | NA | 1425 | 1 | 202 | 0.8065 | 404 |
| 11 | | | | | | | | | |
| 71282- | 287 | 346 | 69 | NA | 1413 | 0.9896 | 186 | 0.875 | 405 |
| 12 | | | | | | | | | |
| 71282- | 288 | 347 | 54 | NA | 1308 | 0.9544 | 214 | 0.92 | 406 |
| 13 | | | | | | | | | |
| 71282- | 289 | 348 | 63 | NA | 1413 | 0.9896 | 159 | 0.8125 | 407 |
| 14 | | | | | | | | | |
| 71282- | 290 | 349 | 63 | NA | 1215 | 0.9132 | 213 | 0.9375 | 408 |
| 15 | | | | | | | | | |
| 71282- | 291 | 350 | 45 | NA | 1089 | 0.8646 | 177 | 0.8542 | 409 |
| 16 | | | | | | | | | |
| 71282- | 292 | 351 | 45 | NA | 1062 | 0.8542 | 168 | 0.8333 | 410 |
| 17 | | | | | | | | | |
| 71282- | 293 | 352 | 57 | NA | 1389 | 0.986 | 241 | 0.98 | 411 |
| 18 | | | | | | | | | |
| 71282- | 294 | 353 | 78 | NA | 1278 | 0.9375 | 193 | 0.8491 | 412 |
| 19 | | | | | | | | | |
| 71282- | 295 | 354 | 72 | NA | 1410 | 0.9828 | 240 | 1 | 413 |
| 20 | | | | | | | | | |

TABLE 11

| | SEQ ID | SEQ ID | SEQ ID | SEQ ID | SEQ ID | SEQ ID | | | |
|---|---|---|---|---|---|---|---|---|---|
| | NO of | NO of | NO of | NO of | NO of | NO of | | | |
| | FWR2_{—} | FWR3_{—} | FWR4_{—} | CDR1_{—} | CDR2_{—} | CDR3_{—} | C_{—} | | |
| Name | IMGT.H | IMGT.H | IMGT.H | IMGT.H | IMGT.H | IMGT.H | CALL.H | CONSCOUNT.H | UMICOUNT.H |
| 71281- | 441 | 500 | 559 | 618 | 677 | 736 | IGHG1 | 1308 | 2 |
| 28 | | | | | | | | | |
| 71281- | 442 | 501 | 560 | 619 | 678 | 737 | IGHG1 | 9630 | 4 |
| 29 | | | | | | | | | |
| 71281- | 443 | 502 | 561 | 620 | 679 | 738 | IGHA2 | 21668 | 3698 |
| 30 | | | | | | | | | |
| 71281- | 444 | 503 | 562 | 621 | 680 | 739 | IGHA1 | 22388 | 13 |
| 31 | | | | | | | | | |
| 71281- | 445 | 504 | 563 | 622 | 681 | 740 | IGHA1 | 31168 | 21 |
| 32 | | | | | | | | | |
| 71281- | 446 | 505 | 564 | 623 | 682 | 741 | IGHG1 | 4910 | 5 |
| 33 | | | | | | | | | |
| 71281- | 447 | 506 | 565 | 624 | 683 | 742 | IGHG2 | 4738 | 11 |
| 34 | | | | | | | | | |
| 71281- | 448 | 507 | 566 | 625 | 684 | 743 | IGHG1 | 2510 | 4 |
| 35 | | | | | | | | | |
| 71281- | 449 | 508 | 567 | 626 | 685 | 744 | IGHA1 | 36760 | 31 |
| 36 | | | | | | | | | |
| 71281- | 450 | 509 | 568 | 627 | 686 | 745 | IGHG1 | 11128 | 12 |
| 37 | | | | | | | | | |
| 71281- | 451 | 510 | 569 | 628 | 687 | 746 | IGHG3 | 3228 | 4 |
| 38 | | | | | | | | | |
| 71281- | 452 | 511 | 570 | 629 | 688 | 747 | IGHG2 | 7884 | 8 |
| 39 | | | | | | | | | |
| 71282-1 | 453 | 512 | 571 | 630 | 689 | 748 | IGHA1 | 3416 | 12 |
| 71282-2 | 454 | 513 | 572 | 631 | 690 | 749 | IGHA1 | 3296 | 12 |
| 71282-3 | 455 | 514 | 573 | 632 | 691 | 750 | IGHG1 | 6686 | 30 |
| 71282-4 | 456 | 515 | 574 | 633 | 692 | 751 | IGHA2 | 9792 | 29 |
| 71282-5 | 457 | 516 | 575 | 634 | 693 | 752 | IGHA2 | 4800 | 17 |
| 71282-6 | 458 | 517 | 576 | 635 | 694 | 753 | IGHG1 | 2754 | 8 |
| 71282-7 | 459 | 518 | 577 | 636 | 695 | 754 | IGHG1 | 2044 | 7 |
| 71282-8 | 460 | 519 | 578 | 637 | 696 | 755 | IGHG1 | 1262 | 5 |
| 71282-9 | 461 | 520 | 579 | 638 | 697 | 756 | IGHA1 | 6546 | 22 |
| 71282- | 462 | 521 | 580 | 639 | 698 | 757 | IGHA1 | 4896 | 19 |
| 10 | | | | | | | | | |
| 71282- | 463 | 522 | 581 | 640 | 699 | 758 | IGHG1 | 1420 | 7 |
| 11 | | | | | | | | | |
| 71282- | 464 | 523 | 582 | 641 | 700 | 759 | IGHA1 | 2686 | 9 |
| 12 | | | | | | | | | |
| 71282- | 465 | 524 | 583 | 642 | 701 | 760 | IGHA1 | 10240 | 31 |
| 13 | | | | | | | | | |
| 71282- | 466 | 525 | 584 | 643 | 702 | 761 | IGHG1 | 304 | 3 |
| 14 | | | | | | | | | |
| 71282- | 467 | 526 | 585 | 644 | 703 | 762 | IGHA2 | 5132 | 19 |
| 15 | | | | | | | | | |
| 71282- | 468 | 527 | 586 | 645 | 704 | 763 | IGHA1 | 11164 | 31 |
| 16 | | | | | | | | | |
| 71282- | 469 | 528 | 587 | 646 | 705 | 764 | IGHG1 | 1668 | 5 |
| 17 | | | | | | | | | |
| 71282- | 470 | 529 | 588 | 647 | 706 | 765 | IGHG3 | 1704 | 5 |
| 18 | | | | | | | | | |
| 71282- | 471 | 530 | 589 | 648 | 707 | 766 | IGHG2 | 2912 | 16 |
| 19 | | | | | | | | | |
| 71282- | 472 | 531 | 590 | 649 | 708 | 767 | IGHA1 | 10480 | 29 |
| 20 | | | | | | | | | |

TABLE 12

| | SEQ ID | SEQ ID | | | SEQ ID | | | | |
|---|---|---|---|---|---|---|---|---|---|
| | NO of | NO of | | | NO of | | | | |
| | JUNCTION_{—} | CDRH3 | | | SEQUENCE_{—} | | IN_{—} | | |
| Name | 10X_AA.H | aa seq | CLONE | CONTIG.L | INPUT.L | FUNCTIONAL.L | FRAME.L | INDELS.L | LOCUS.L |
| 71281- | 795 | 854 | 4251 | contig_1 | 913 | T | T | F | IGK |
| 28 | | | | | | | | | |
| 71281- | 796 | 855 | 4350 | contig_1 | 914 | T | T | F | IGL |
| 29 | | | | | | | | | |
| 71281- | 797 | 856 | 4619 | contig_1 | 915 | T | T | F | IGK |
| 30 | | | | | | | | | |
| 71281- | 798 | 857 | 4726 | contig_1 | 916 | T | T | F | IGL |
| 31 | | | | | | | | | |
| 71281- | 799 | 858 | 4850 | contig_1 | 917 | T | T | F | IGL |
| 32 | | | | | | | | | |
| 71281- | 800 | 859 | 5059 | contig_1 | 918 | T | T | F | IGK |
| 33 | | | | | | | | | |
| 71281- | 801 | 860 | 5235 | contig_1 | 919 | T | T | F | IGK |
| 34 | | | | | | | | | |
| 71281- | 802 | 861 | 5946 | contig_1 | 920 | T | T | F | IGK |
| 35 | | | | | | | | | |
| 71281- | 803 | 862 | 6432 | contig_2 | 921 | T | T | F | IGK |
| 36 | | | | | | | | | |
| 71281- | 804 | 863 | 6528 | contig_1 | 922 | T | T | F | IGK |
| 37 | | | | | | | | | |
| 71281- | 805 | 864 | 6543 | contig_1 | 923 | T | T | F | IGL |
| 38 | | | | | | | | | |
| 71281- | 806 | 865 | 6580 | contig_1 | 924 | T | T | F | IGK |
| 39 | | | | | | | | | |
| 71282-1 | 807 | 866 | 121 | contig_1 | 925 | T | T | F | IGL |
| 71282-2 | 808 | 867 | 124 | contig_1 | 926 | T | T | F | IGK |
| 71282-3 | 809 | 868 | 131 | contig_1 | 927 | T | T | F | IGL |
| 71282-4 | 810 | 869 | 632 | contig_2 | 928 | T | T | F | IGK |
| 71282-5 | 811 | 870 | 785 | contig_1 | 929 | T | T | F | IGL |
| 71282-6 | 812 | 871 | 861 | contig_1 | 930 | T | T | F | IGL |
| 71282-7 | 813 | 872 | 1105 | contig_1 | 931 | T | T | F | IGK |
| 71282-8 | 814 | 873 | 1135 | contig_1 | 932 | T | T | F | IGK |
| 71282-9 | 815 | 874 | 1153 | contig_1 | 933 | T | T | F | IGK |
| 71282- | 816 | 875 | 1719 | contig_2 | 934 | T | T | F | IGK |
| 10 | | | | | | | | | |
| 71282- | 817 | 876 | 1739 | contig_1 | 935 | T | T | F | IGL |
| 11 | | | | | | | | | |
| 71282- | 818 | 877 | 1783 | contig_1 | 936 | T | T | F | IGK |
| 12 | | | | | | | | | |
| 71282- | 819 | 878 | 1859 | contig_1 | 937 | T | T | F | IGL |
| 13 | | | | | | | | | |
| 71282- | 820 | 879 | 2253 | contig_1 | 938 | T | T | F | IGL |
| 14 | | | | | | | | | |
| 71282- | 821 | 880 | 2301 | contig_1 | 939 | T | T | F | IGK |
| 15 | | | | | | | | | |
| 71282- | 822 | 881 | 2418 | contig_2 | 940 | T | T | T | IGL |
| 16 | | | | | | | | | |
| 71282- | 823 | 882 | 2522 | contig_1 | 941 | T | T | F | IGK |
| 17 | | | | | | | | | |
| 71282- | 824 | 883 | 2783 | contig_1 | 942 | T | T | F | IGK |
| 18 | | | | | | | | | |
| 71282- | 825 | 884 | 3021 | contig_2 | 943 | T | T | F | IGK |
| 19 | | | | | | | | | |
| 71282- | 826 | 885 | 3041 | contig_2 | 944 | T | T | F | IGL |
| 20 | | | | | | | | | |

TABLE 13

| | | | SEQ ID | SEQ ID | SEQ ID | | | | |
|---|---|---|---|---|---|---|---|---|---|
| | | | NO of | NO of | NO of | SEQ ID | | | |
| | V_{—} | J_{—} | SEQUENCE_{—} | LC seq | SEQUENCE_{—} | NO of | JUNCTION_{—} | GERMLINE_{—} | V_{—} |
| Name | CALL.L | CALL.L | VJ.L | VJ aa | IMGT.L | JUNCTION.L | LENGTH.L | IMGT.L | SCORE.L |
| 71281- | IGKV4- | IGKJ4 | 972 | 1031 | 1090 | 1149 | 33 | NA | 1449 |
| 28 | 1 | | | | | | | | |
| 71281- | IGLV1- | IGLJ3 | 973 | 1032 | 1091 | 1150 | 39 | NA | 1413 |
| 29 | 40 | | | | | | | | |
| 71281- | IGKV1- | IGKJ4 | 974 | 1033 | 1092 | 1151 | 33 | NA | 1107 |
| 30 | 33 | | | | | | | | |
| 71281- | IGLV3- | IGLJ2 | 975 | 1034 | 1093 | 1152 | 39 | NA | 1314 |
| 31 | 21 | | | | | | | | |
| 71281- | IGLV2- | IGLJ2 | 976 | 1035 | 1094 | 1153 | 33 | NA | 1395 |
| 32 | 14 | | | | | | | | |
| 71281- | IGKV1- | IGKJ2 | 977 | 1036 | 1095 | 1154 | 27 | NA | 1287 |
| 33 | 5 | | | | | | | | |
| 71281- | IGKV1- | IGKJ1 | 978 | 1037 | 1096 | 1155 | 33 | NA | 1269 |
| 34 | 9 | | | | | | | | |
| 71281- | IGKV1- | IGKJ2 | 979 | 1038 | 1097 | 1156 | 33 | NA | 1368 |
| 35 | 33 | | | | | | | | |
| 71281- | IGKV3- | IGKJ2 | 980 | 1039 | 1098 | 1157 | 33 | NA | 1266 |
| 36 | 20 | | | | | | | | |
| 71281- | IGKV1- | IGKJ5 | 981 | 1040 | 1099 | 1158 | 33 | NA | 1350 |
| 37 | 33 | | | | | | | | |
| 71281- | IGLV1- | IGLJ3 | 982 | 1041 | 1100 | 1159 | 39 | NA | 1353 |
| 38 | 44 | | | | | | | | |
| 71281- | IGKV1- | IGKJ2 | 983 | 1042 | 1101 | 1160 | 33 | NA | 1296 |
| 39 | 17 | | | | | | | | |
| 71282- | IGLV1- | IGLJ2 | 984 | 1043 | 1102 | 1161 | 39 | NA | 1422 |
| 1 | 40 | | | | | | | | |
| 71282- | IGKV3- | IGKJ2 | 985 | 1044 | 1103 | 1162 | 33 | NA | 1332 |
| 2 | 11 | | | | | | | | |
| 71282- | IGLV1- | IGLJ3 | 986 | 1045 | 1104 | 1163 | 39 | NA | 1359 |
| 3 | 40 | | | | | | | | |
| 71282- | IGKV1- | IGKJ1 | 987 | 1046 | 1105 | 1164 | 33 | NA | 1314 |
| 4 | 17 | | | | | | | | |
| 71282- | IGLV3- | IGLJ2 | 988 | 1047 | 1106 | 1165 | 39 | NA | 1278 |
| 5 | 25 | | | | | | | | |
| 71282- | IGLV1- | IGLJ3 | 989 | 1048 | 1107 | 1166 | 39 | NA | 1407 |
| 6 | 36 | | | | | | | | |
| 71282- | IGKV3- | IGKJ3 | 990 | 1049 | 1108 | 1167 | 30 | NA | 1383 |
| 7 | 20 | | | | | | | | |
| 71282- | IGKV1- | IGKJ2 | 991 | 1050 | 1109 | 1168 | 36 | NA | 1359 |
| 8 | 39 | | | | | | | | |
| 71282- | IGKV3- | IGKJ2 | 992 | 1051 | 1110 | 1169 | 33 | NA | 1257 |
| 9 | 20 | | | | | | | | |
| 71282- | IGKV3- | IGKJ2 | 993 | 1052 | 1111 | 1170 | 36 | NA | 1356 |
| 10 | 20 | | | | | | | | |
| 71282- | IGLV4- | IGLJ3 | 994 | 1053 | 1112 | 1171 | 33 | NA | 1425 |
| 11 | 69 | | | | | | | | |
| 71282- | IGKV1- | IGKJ1 | 995 | 1054 | 1113 | 1172 | 33 | NA | 1359 |
| 12 | 39 | | | | | | | | |
| 71282- | IGLV3- | IGLJ1 | 996 | 1055 | 1114 | 1173 | 36 | NA | 1215 |
| 13 | 21 | | | | | | | | |
| 71282- | IGLV1- | IGLJ1 | 997 | 1056 | 1115 | 1174 | 39 | NA | 1371 |
| 14 | 47 | | | | | | | | |
| 71282- | IGKV1- | IGKJ4 | 998 | 1057 | 1116 | 1175 | 30 | NA | 1206 |
| 15 | 39 | | | | | | | | |
| 71282- | IGLV3- | IGLJ2 | 999 | 1058 | 1117 | 1176 | 33 | NA | 1098 |
| 16 | 1 | | | | | | | | |
| 71282- | IGKV3- | IGKJ2 | 1000 | 1059 | 1118 | 1177 | 30 | NA | 1203 |
| 17 | 20 | | | | | | | | |
| 71282- | IGKV2D- | IGKJ2 | 1001 | 1060 | 1119 | 1178 | 33 | NA | 1434 |
| 18 | 29 | | | | | | | | |
| 71282- | IGKV1- | IGKJ2 | 1002 | 1061 | 1120 | 1179 | 30 | NA | 1251 |
| 19 | 5 | | | | | | | | |

TABLE 14

| | | | | SEQ ID | SEQ ID | SEQ ID | SEQ ID | SEQ ID | SEQ ID |
|---|---|---|---|---|---|---|---|---|---|
| | | | | NO of | NO of | NO of | NO of | NO of | NO of |
| | V_{—} | J_{—} | J_{—} | FWR1_{—} | FWR2_{—} | FWR3_{—} | FWR4_{—} | CDR1_{—} | CDR2_{—} |
| Name | IDENTITY.L | SCORE.L | IDENTITY.L | IMGT.L | IMGT.L | IMGT.LL | IMGT.LL | IMGT.L | IMGT.L |
| 71281- | 0.9865 | 167 | 0.9459 | 1208 | 1267 | 1326 | 1385 | 1444 | 1503 |
| 28 | | | | | | | | | |
| 71281- | 0.9896 | 180 | 1 | 1209 | 1268 | 1327 | 1386 | 1445 | 1504 |
| 29 | | | | | | | | | |
| 71281- | 0.8853 | 172 | 0.9474 | 1210 | 1269 | 1328 | 1387 | 1446 | 1505 |
| 30 | | | | | | | | | |
| 71281- | 0.9677 | 176 | 0.973 | 1211 | 1270 | 1329 | 1388 | 1447 | 1506 |
| 31 | | | | | | | | | |
| 71281- | 0.9826 | 176 | 0.973 | 1212 | 1271 | 1330 | 1389 | 1448 | 1507 |
| 32 | | | | | | | | | |
| 71281- | 0.957 | 155 | 1 | 1213 | 1272 | 1331 | 1390 | 1449 | 1508 |
| 33 | | | | | | | | | |
| 71281- | 0.9498 | 180 | 1 | 1214 | 1273 | 1332 | 1391 | 1450 | 1509 |
| 34 | | | | | | | | | |
| 71281- | 0.9892 | 180 | 1 | 1215 | 1274 | 1333 | 1392 | 1451 | 1510 |
| 35 | | | | | | | | | |
| 71281- | 0.9433 | 181 | 0.9737 | 1216 | 1275 | 1334 | 1393 | 1452 | 1511 |
| 36 | | | | | | | | | |
| 71281- | 0.9821 | 163 | 0.9211 | 1217 | 1276 | 1335 | 1394 | 1453 | 1512 |
| 37 | | | | | | | | | |
| 71281- | 0.9719 | 171 | 0.9722 | 1218 | 1277 | 1336 | 1395 | 1454 | 1513 |
| 38 | | | | | | | | | |
| 71281- | 0.9606 | 181 | 0.9737 | 1219 | 1278 | 1337 | 1396 | 1455 | 1514 |
| 39 | | | | | | | | | |
| 71282-1 | 0.9931 | 166 | 0.9714 | 1220 | 1279 | 1338 | 1397 | 1456 | 1515 |
| 71282-2 | 0.9749 | 135 | 0.8611 | 1221 | 1280 | 1339 | 1398 | 1457 | 1516 |
| 71282-3 | 0.9688 | 166 | 0.9714 | 1222 | 1281 | 1340 | 1399 | 1458 | 1517 |
| 71282-4 | 0.9677 | 167 | 0.9459 | 1223 | 1282 | 1341 | 1400 | 1459 | 1518 |
| 71282-5 | 0.9534 | 158 | 0.9189 | 1224 | 1283 | 1342 | 1401 | 1460 | 1519 |
| 71282-6 | 0.993 | 181 | 0.9737 | 1225 | 1284 | 1343 | 1402 | 1461 | 1520 |
| 71282-7 | 0.9894 | 167 | 0.9459 | 1226 | 1285 | 1344 | 1403 | 1462 | 1521 |
| 71282-8 | 0.9857 | 195 | 1 | 1227 | 1286 | 1345 | 1404 | 1463 | 1522 |
| 71282-9 | 0.9397 | 167 | 0.9459 | 1228 | 1287 | 1346 | 1405 | 1464 | 1523 |
| 71282- | 0.9787 | 195 | 1 | 1229 | 1288 | 1347 | 1406 | 1465 | 1524 |
| 10 | | | | | | | | | |
| 71282- | 0.983 | 171 | 0.9722 | 1230 | 1289 | 1348 | 1407 | 1466 | 1525 |
| 11 | | | | | | | | | |
| 71282- | 0.9857 | 162 | 0.9444 | 1231 | 1290 | 1349 | 1408 | 1467 | 1526 |
| 12 | | | | | | | | | |
| 71282- | 0.9283 | 134 | 0.8824 | 1232 | 1291 | 1350 | 1409 | 1468 | 1527 |
| 13 | | | | | | | | | |
| 71282- | 0.9789 | 185 | 1 | 1233 | 1292 | 1351 | 1410 | 1469 | 1528 |
| 14 | | | | | | | | | |
| 71282- | 0.9247 | 157 | 0.9429 | 1234 | 1293 | 1352 | 1411 | 1470 | 1529 |
| 15 | | | | | | | | | |
| 71282- | 0.8817 | 153 | 0.9167 | 1235 | 1294 | 1353 | 1412 | 1471 | 1530 |
| 16 | | | | | | | | | |
| 71282- | 0.9184 | 168 | 0.9231 | 1236 | 1295 | 1354 | 1413 | 1472 | 1531 |
| 17 | | | | | | | | | |
| 71282- | 0.9864 | 195 | 1 | 1237 | 1296 | 1355 | 1414 | 1473 | 1532 |
| 18 | | | | | | | | | |
| 71282- | 0.9427 | 150 | 0.8718 | 1238 | 1297 | 1356 | 1415 | 1474 | 1533 |
| 19 | | | | | | | | | |
| 71282- | 0.9857 | 176 | 0.973 | 1239 | 1298 | 1357 | 1416 | 1475 | 1534 |
| 20 | | | | | | | | | |

TABLE 15

| | SEQ ID | | | SEQ ID | | | | | | |
|---|---|---|---|---|---|---|---|---|---|---|
| | NO of | | | NO of | SEQ ID | SEQ ID | SEQ ID | SEQ ID | SEQ ID | SEQ ID |
| | CDR3_{—} | C_{—} | | JUNCTION_{—} | NO of | NO of | NO of | NO of | NO of | NO of |
| Name | IMGT.L | CALL.L | CONSCOUNT.L | 10X_AA.L | CDRH1 | CDRH2 | CDRH3 | CDRL1 | CDRL2 | CDRL3 |
| 71281-28 | 1562 | IGKC | 9060 | 1621 | 1680 | 1739 | 1798 | 1857 | 1916 | 1975 |
| 71281-29 | 1563 | IGLC2 | 26240 | 1622 | 1681 | 1740 | 1799 | 1858 | 1917 | 1976 |
| 71281-30 | 1564 | IGKC | 56410 | 1623 | 1682 | 1741 | 1800 | 1859 | 1918 | 1977 |
| 71281-31 | 1565 | IGLC2 | 14754 | 1624 | 1683 | 1742 | 1801 | 1860 | 1919 | 1978 |
| 71281-32 | 1566 | IGLC2 | 37210 | 1625 | 1684 | 1743 | 1802 | 1861 | 1920 | 1979 |
| 71281-33 | 1567 | IGKC | 25244 | 1626 | 1685 | 1744 | 1803 | 1862 | 1921 | 1980 |
| 71281-34 | 1568 | IGKC | 40240 | 1627 | 1686 | 1745 | 1804 | 1863 | 1922 | 1981 |
| 71281-35 | 1569 | IGKC | 42414 | 1628 | 1687 | 1746 | 1805 | 1864 | 1923 | 1982 |
| 71281-36 | 1570 | IGKC | 32176 | 1629 | 1688 | 1747 | 1806 | 1865 | 1924 | 1983 |
| 71281-37 | 1571 | IGKC | 15316 | 1630 | 1689 | 1748 | 1807 | 1866 | 1925 | 1984 |
| 71281-38 | 1572 | IGLC2 | 14670 | 1631 | 1690 | 1749 | 1808 | 1867 | 1926 | 1985 |
| 71281-39 | 1573 | IGKC | 24994 | 1632 | 1691 | 1750 | 1809 | 1868 | 1927 | 1986 |
| 71282-1 | 1574 | IGLC2 | 2776 | 1633 | 1692 | 1751 | 1810 | 1869 | 1928 | 1987 |
| 71282-2 | 1575 | IGKC | 18444 | 1634 | 1693 | 1752 | 1811 | 1870 | 1929 | 1988 |
| 71282-3 | 1576 | IGLC2 | 48404 | 1635 | 1694 | 1753 | 1812 | 1871 | 1930 | 1989 |
| 71282-4 | 1577 | IGKC | 14058 | 1636 | 1695 | 1754 | 1813 | 1872 | 1931 | 1990 |
| 71282-5 | 1578 | IGLC2 | 14646 | 1637 | 1696 | 1755 | 1814 | 1873 | 1932 | 1991 |
| 71282-6 | 1579 | IGLC3 | 20506 | 1638 | 1697 | 1756 | 1815 | 1874 | 1933 | 1992 |
| 71282-7 | 1580 | IGKC | 15224 | 1639 | 1698 | 1757 | 1816 | 1875 | 1934 | 1993 |
| 71282-8 | 1581 | IGKC | 9006 | 1640 | 1699 | 1758 | 1817 | 1876 | 1935 | 1994 |
| 71282-9 | 1582 | IGKC | 7474 | 1641 | 1700 | 1759 | 1818 | 1877 | 1936 | 1995 |
| 71282-10 | 1583 | IGKC | 26756 | 1642 | 1701 | 1760 | 1819 | 1878 | 1937 | 1996 |
| 71282-11 | 1584 | IGLC2 | 5850 | 1643 | 1702 | 1761 | 1820 | 1879 | 1938 | 1997 |
| 71282-12 | 1585 | IGKC | 8040 | 1644 | 1703 | 1762 | 1821 | 1880 | 1939 | 1998 |
| 71282-13 | 1586 | IGLC1 | 5928 | 1645 | 1704 | 1763 | 1822 | 1881 | 1940 | 1999 |
| 71282-14 | 1587 | IGLC1 | 1740 | 1646 | 1705 | 1764 | 1823 | 1882 | 1941 | 2000 |
| 71282-15 | 1588 | IGKC | 18966 | 1647 | 1706 | 1765 | 1824 | 1883 | 1942 | 2001 |
| 71282-16 | 1589 | IGLC2 | 10376 | 1648 | 1707 | 1766 | 1825 | 1884 | 1943 | 2002 |
| 71282-17 | 1590 | IGKC | 19296 | 1649 | 1708 | 1767 | 1826 | 1885 | 1944 | 2003 |
| 71282-18 | 1591 | IGKC | 23824 | 1650 | 1709 | 1768 | 1827 | 1886 | 1945 | 2004 |
| 71282-19 | 1592 | IGKC | 32780 | 1651 | 1710 | 1769 | 1828 | 1887 | 1946 | 2005 |
| 71282-20 | 1593 | IGLC2 | 26438 | 1652 | 1711 | 1770 | 1829 | 1888 | 1947 | 2006 |

TABLE 16

| | SEQ ID | | | | SEQ ID | | | |
|---|---|---|---|---|---|---|---|---|
| | NO of | SEQ ID | SEQ ID | SEQ ID | NO of | SEQ ID | SEQ ID | SEQ ID |
| | HC seq | NO of | NO of | NO of | LC seq | NO of | NO of | NO of |
| Name | VDJ aa | CDRH1 | CDRH2 | CDRH3 | VJ aa | CDRL1 | CDRL2 | CDRL3 |
| 71281-1 | 178 | 1653 | 1712 | 1771 | 1004 | 1830 | 1889 | 1948 |
| 71281-2 | 179 | 1654 | 1713 | 1772 | 1005 | 1831 | 1890 | 1949 |
| 71281-3 | 180 | 1655 | 1714 | 1773 | 1006 | 1832 | 1891 | 1950 |
| 71281-4 | 181 | 1656 | 1715 | 1774 | 1007 | 1833 | 1892 | 1951 |
| 71281-5 | 182 | 1657 | 1716 | 1775 | 1008 | 1834 | 1893 | 1952 |
| 71281-6 | 183 | 1658 | 1717 | 1776 | 1009 | 1835 | 1894 | 1953 |
| 71281-7 | 184 | 1659 | 1718 | 1777 | 1010 | 1836 | 1895 | 1954 |
| 71281-8 | 185 | 1660 | 1719 | 1778 | 1011 | 1837 | 1896 | 1955 |
| 71281-9 | 186 | 1661 | 1720 | 1779 | 1012 | 1838 | 1897 | 1956 |
| 71281-10 | 187 | 1662 | 1721 | 1780 | 1013 | 1839 | 1898 | 1957 |
| 71281-11 | 188 | 1663 | 1722 | 1781 | 1014 | 1840 | 1899 | 1958 |
| 71281-12 | 189 | 1664 | 1723 | 1782 | 1015 | 1841 | 1900 | 1959 |
| 71281-13 | 190 | 1665 | 1724 | 1783 | 1016 | 1842 | 1901 | 1960 |
| 71281-14 | 191 | 1666 | 1725 | 1784 | 1017 | 1843 | 1902 | 1961 |
| 71281-15 | 192 | 1667 | 1726 | 1785 | 1018 | 1844 | 1903 | 1962 |
| 71281-16 | 193 | 1668 | 1727 | 1786 | 1019 | 1845 | 1904 | 1963 |
| 71281-17 | 194 | 1669 | 1728 | 1787 | 1020 | 1846 | 1905 | 1964 |
| 71281-18 | 195 | 1670 | 1729 | 1788 | 1021 | 1847 | 1906 | 1965 |
| 71281-19 | 196 | 1671 | 1730 | 1789 | 1022 | 1848 | 1907 | 1966 |
| 71281-20 | 197 | 1672 | 1731 | 1790 | 1023 | 1849 | 1908 | 1967 |
| 71281-21 | 198 | 1673 | 1732 | 1791 | 1024 | 1850 | 1909 | 1968 |
| 71281-22 | 199 | 1674 | 1733 | 1792 | 1025 | 1851 | 1910 | 1969 |
| 71281-23 | 200 | 1675 | 1734 | 1793 | 1026 | 1852 | 1911 | 1970 |
| 71281-24 | 201 | 1676 | 1735 | 1794 | 1027 | 1853 | 1912 | 1971 |
| 71281-25 | 202 | 1677 | 1736 | 1795 | 1028 | 1854 | 1913 | 1972 |
| 71281-26 | 203 | 1678 | 1737 | 1796 | 1029 | 1855 | 1914 | 1973 |
| 71281-27 | 204 | 1679 | 1738 | 1797 | 1030 | 1856 | 1915 | 1974 |
| 71281-28 | 205 | 1680 | 1739 | 1798 | 1031 | 1857 | 1916 | 1975 |
| 71281-29 | 206 | 1681 | 1740 | 1799 | 1032 | 1858 | 1917 | 1976 |
| 71281-30 | 207 | 1682 | 1741 | 1800 | 1033 | 1859 | 1918 | 1977 |
| 71281-31 | 208 | 1683 | 1742 | 1801 | 1034 | 1860 | 1919 | 1978 |
| 71281-32 | 209 | 1684 | 1743 | 1802 | 1035 | 1861 | 1920 | 1979 |
| 71281-33 | 210 | 1685 | 1744 | 1803 | 1036 | 1862 | 1921 | 1980 |
| 71281-34 | 211 | 1686 | 1745 | 1804 | 1037 | 1863 | 1922 | 1981 |
| 71281-35 | 212 | 1687 | 1746 | 1805 | 1038 | 1864 | 1923 | 1982 |
| 71281-36 | 213 | 1688 | 1747 | 1806 | 1039 | 1865 | 1924 | 1983 |
| 71281-37 | 214 | 1689 | 1748 | 1807 | 1040 | 1866 | 1925 | 1984 |
| 71281-38 | 215 | 1690 | 1749 | 1808 | 1041 | 1867 | 1926 | 1985 |
| 71281-39 | 216 | 1691 | 1750 | 1809 | 1042 | 1868 | 1927 | 1986 |
| 71282-1 | 217 | 1692 | 1751 | 1810 | 1043 | 1869 | 1928 | 1987 |
| 71282-2 | 218 | 1693 | 1752 | 1811 | 1044 | 1870 | 1929 | 1988 |
| 71282-3 | 219 | 1694 | 1753 | 1812 | 1045 | 1871 | 1930 | 1989 |
| 71282-4 | 220 | 1695 | 1754 | 1813 | 1046 | 1872 | 1931 | 1990 |
| 71282-5 | 221 | 1696 | 1755 | 1814 | 1047 | 1873 | 1932 | 1991 |
| 71282-6 | 222 | 1697 | 1756 | 1815 | 1048 | 1874 | 1933 | 1992 |
| 71282-7 | 223 | 1698 | 1757 | 1816 | 1049 | 1875 | 1934 | 1993 |
| 71282-8 | 224 | 1699 | 1758 | 1817 | 1050 | 1876 | 1935 | 1994 |
| 71282-9 | 225 | 1700 | 1759 | 1818 | 1051 | 1877 | 1936 | 1995 |
| 71282-10 | 226 | 1701 | 1760 | 1819 | 1052 | 1878 | 1937 | 1996 |
| 71282-11 | 227 | 1702 | 1761 | 1820 | 1053 | 1879 | 1938 | 1997 |
| 71282-12 | 228 | 1703 | 1762 | 1821 | 1054 | 1880 | 1939 | 1998 |
| 71282-13 | 229 | 1704 | 1763 | 1822 | 1055 | 1881 | 1940 | 1999 |
| 71282-14 | 230 | 1705 | 1764 | 1823 | 1056 | 1882 | 1941 | 2000 |
| 71282-15 | 231 | 1706 | 1765 | 1824 | 1057 | 1883 | 1942 | 2001 |
| 71282-16 | 232 | 1707 | 1766 | 1825 | 1058 | 1884 | 1943 | 2002 |
| 71282-17 | 233 | 1708 | 1767 | 1826 | 1059 | 1885 | 1944 | 2003 |
| 71282-18 | 234 | 1709 | 1768 | 1827 | 1060 | 1886 | 1945 | 2004 |
| 71282-19 | 235 | 1710 | 1769 | 1828 | 1061 | 1887 | 1946 | 2005 |
| 71282-20 | 236 | 1711 | 1770 | 1829 | 1062 | 1888 | 1947 | 2006 |

1. A recombinant antibody comprising a light chain variable region (VL) that comprises a light chain complementarity determining region (CDRL)1, a CDRL2, and a CDRL3 and a heavy chain variable region (VH) that comprises a heavy chain complementarity determining region (CDRH)1, CDRH2, CDRH3, wherein:
CDRL3 comprises a sequence at least 60% identical to a sequence selected from SEQ ID NOs: 1948-2006; and
CDRH3 comprises a sequence at least 60% identical to a sequence selected from SEQ ID NOs: 1771-1829.

2. (canceled)

3. (canceled)

4. The recombinant antibody of claim 1, wherein:
CDRL2 comprises a sequence at least 60% identical to a sequence selected from SEQ ID NOs: 1889-1947; and
CDRH2 comprises a sequence at least 60% identical to a sequence selected from SEQ ID NOs: 1712-1770.

5. (canceled)

6. (canceled)

7. The recombinant antibody of claim 1, wherein:
CDRL1 comprises a sequence at least 60% identical to a sequence selected from SEQ ID NOs: 1830-1888; and
CDRH1 comprises a sequence at least 60% identical to a sequence selected from SEQ ID NOs: 1653-1711.

8. (canceled)

9. (canceled)

10. The recombinant antibody of claim 1, wherein VH comprises a sequence at least 60% identical to a sequence selected from SEQ ID NOs: 178-236.

11. The recombinant antibody of claim 1, wherein VL comprises a sequence at least 60% identical to a sequence selected from SEQ ID NOs: 1004-1062.

12. The recombinant antibody of claim 1, wherein:
CDRL3 comprises a sequence at least 60% identical to a sequence selected from SEQ ID NO: 1950, SEQ ID NO: 1953, SEQ ID NO: 1967, SEQ ID NO: 1969, SEQ ID NO: 1973, SEQ ID NO: 1978, SEQ ID NO: 1979, SEQ ID NO: 1980, SEQ ID NO: 1982, SEQ ID NO: 1983, SEQ ID NO: 1984, SEQ ID NO: 1990, SEQ ID NO: 1992, SEQ ID NO: 1993, SEQ ID NO: 1996, SEQ ID NO: 2000, or SEQ ID NO: 2004; and
CDRH3 comprises a sequence at least 60% identical to a sequence selected from SEQ ID NO: 1773, SEQ ID NO: 1776, SEQ ID NO: 1790, SEQ ID NO: 1792, SEQ ID NO: 1796, SEQ ID NO: 1801, SEQ ID NO: 1802, SEQ ID NO: 1803, SEQ ID NO: 1805, SEQ ID NO: 1806, SEQ ID NO: 1807, SEQ ID NO: 1813, SEQ ID NO: 1815, SEQ ID NO: 1816, SEQ ID NO: 1819, SEQ ID NO: 1823, or SEQ ID NO: 1827.

13. The recombinant antibody of claim 1, wherein CDRL3 comprises at least one amino acid substitution when compared to SEQ ID NO: 1950, SEQ ID NO: 1953, SEQ ID NO: 1967, SEQ ID NO: 1969, SEQ ID NO: 1973, SEQ ID NO: 1978, SEQ ID NO: 1979, SEQ ID NO: 1980, SEQ ID NO: 1982, SEQ ID NO: 1983, SEQ ID NO: 1984, SEQ ID NO: 1990, SEQ ID NO: 1992, SEQ ID NO: 1993, SEQ ID NO: 1996, SEQ ID NO: 2000, or SEQ ID NO: 2004.

14. The recombinant antibody of claim 1, wherein CDRH3 comprises at least one amino acid substitution when compared to SEQ ID NO: 1773, SEQ ID NO: 1776, SEQ ID NO: 1790, SEQ ID NO: 1792, SEQ ID NO: 1796, SEQ ID NO: 1801, SEQ ID NO: 1802, SEQ ID NO: 1803, SEQ ID NO: 1805, SEQ ID NO: 1806, SEQ ID NO: 1807, SEQ ID NO: 1813, SEQ ID NO: 1815, SEQ ID NO: 1816, SEQ ID NO: 1819, SEQ ID NO: 1823, or SEQ ID NO: 1827.

15. The recombinant antibody of claim 1, wherein:
CDRL2 comprises a sequence at least 60% identical to a sequence selected from SEQ ID NO: 1891, SEQ ID NO: 1894, SEQ ID NO: 1908, SEQ ID NO: 1910, SEQ ID NO: 1914, SEQ ID NO: 1919, SEQ ID NO: 1920, SEQ ID NO: 1921, SEQ ID NO: 1923, SEQ ID NO: 1924, SEQ ID NO: 1925, SEQ ID NO: 1931, SEQ ID NO: 1933, SEQ ID NO: 1934, SEQ ID NO: 1937, SEQ ID NO: 1941, or SEQ ID NO: 1945; and
CDRH2 comprises a sequence at least 60% identical to a sequence selected from SEQ ID NO: 1714, SEQ ID NO: 1717, SEQ ID NO: 1731, SEQ ID NO: 1733, SEQ ID NO: 1737, SEQ ID NO: 1742, SEQ ID NO: 1743, SEQ ID NO: 1744, SEQ ID NO: 1746, SEQ ID NO: 1747, SEQ ID NO: 1748, SEQ ID NO: 1754, SEQ ID NO: 1756, SEQ ID NO: 1757, SEQ ID NO: 1760, SEQ ID NO: 1764, or SEQ ID NO: 1768.

16. The recombinant antibody of claim 1, wherein CDRL2 comprises at least one amino acid substitution when compared to SEQ ID NO: 1891, SEQ ID NO: 1894, SEQ ID NO: 1908, SEQ ID NO: 1910, SEQ ID NO: 1914, SEQ ID NO: 1919, SEQ ID NO: 1920, SEQ ID NO: 1921, SEQ ID NO: 1923, SEQ ID NO: 1924, SEQ ID NO: 1925, SEQ ID NO: 1931, SEQ ID NO: 1933, SEQ ID NO: 1934, SEQ ID NO: 1937, SEQ ID NO: 1941, or SEQ ID NO: 1945.

17. The recombinant antibody of claim 1, wherein CDRH2 comprises at least one amino acid substitution when compared to SEQ ID NO: 1714, SEQ ID NO: 1717, SEQ ID NO: 1731, SEQ ID NO: 1733, SEQ ID NO: 1737, SEQ ID NO: 1742, SEQ ID NO: 1743, SEQ ID NO: 1744, SEQ ID NO: 1746, SEQ ID NO: 1747, SEQ ID NO: 1748, SEQ ID NO: 1754, SEQ ID NO: 1756, SEQ ID NO: 1757, SEQ ID NO: 1760, SEQ ID NO: 1764, or SEQ ID NO: 1768.

18. The recombinant antibody of claim 1, wherein:
CDRL1 comprises a sequence at least 60% identical to a sequence selected from SEQ ID NO: 1832, SEQ ID NO: 1835, SEQ ID NO: 1849, SEQ ID NO: 1851, SEQ ID NO: 1855, SEQ ID NO: 1860, SEQ ID NO: 1861, SEQ ID NO: 1862, SEQ ID NO: 1864, SEQ ID NO: 1865, SEQ ID NO: 1866, SEQ ID NO: 1872, SEQ ID NO: 1874, SEQ ID NO: 1875, SEQ ID NO: 1878, SEQ ID NO: 1882, or SEQ ID NO: 1886; and
CDRH1 comprises a sequence at least 60% identical to a sequence selected from SEQ ID NO: 1655, SEQ ID NO: 1658, SEQ ID NO: 1672, SEQ ID NO: 1674, SEQ ID NO: 1678, SEQ ID NO: 1683, SEQ ID NO: 1684, SEQ ID NO: 1685, SEQ ID NO: 1687, SEQ ID NO: 1688, SEQ ID NO: 1689, SEQ ID NO: 1695, SEQ ID NO: 1697, SEQ ID NO: 1698, SEQ ID NO: 1701, SEQ ID NO: 1705, or SEQ ID NO: 1709.

19. The recombinant antibody of claim 1, wherein CDRL1 comprises at least one amino acid substitution when compared to SEQ ID NO: 1832, SEQ ID NO: 1835, SEQ ID NO: 1849, SEQ ID NO: 1851, SEQ ID NO: 1855, SEQ ID NO: 1860, SEQ ID NO: 1861, SEQ ID NO: 1862, SEQ ID NO: 1864, SEQ ID NO: 1865, SEQ ID NO: 1866, SEQ ID NO: 1872, SEQ ID NO: 1874, SEQ ID NO: 1875, SEQ ID NO: 1878, SEQ ID NO: 1882, or SEQ ID NO: 1886.

20. The recombinant antibody of claim 1, wherein CDRH1 comprises at least one amino acid substitution when compared to SEQ ID NO: 1655, SEQ ID NO: 1658, SEQ ID NO: 1672, SEQ ID NO: 1674, SEQ ID NO: 1678, SEQ ID NO: 1683, SEQ ID NO: 1684, SEQ ID NO: 1685, SEQ ID NO: 1687, SEQ ID NO: 1688, SEQ ID NO: 1689, SEQ ID NO: 1695, SEQ ID NO: 1697, SEQ ID NO: 1698, SEQ ID NO: 1701, SEQ ID NO: 1705, or SEQ ID NO: 1709.

21. The recombinant antibody of claim 1, wherein VH comprises a sequence at least 60% identical to a sequence selected from SEQ ID NO: 180, SEQ ID NO: 183, SEQ ID NO: 197, SEQ ID NO: 199, SEQ ID NO: 203, SEQ ID NO: 208, SEQ ID NO: 209, SEQ ID NO: 210, SEQ ID NO: 212, SEQ ID NO: 213, SEQ ID NO: 214, SEQ ID NO: 220, SEQ ID NO: 222, SEQ ID NO: 223, SEQ ID NO: 226, SEQ ID NO: 230, or SEQ ID NO: 234.

22. The recombinant antibody of claim 1, wherein VL comprises a sequence at least 60% identical to a sequence selected from SEQ ID NO: 1006, SEQ ID NO: 1009, SEQ ID NO: 1023, SEQ ID NO: 1025, SEQ ID NO: 1029, SEQ ID NO: 1034, SEQ ID NO: 1035, SEQ ID NO: 1036, SEQ ID NO: 1038, SEQ ID NO: 1039, SEQ ID NO: 1040, SEQ ID NO: 1046, SEQ ID NO: 1048, SEQ ID NO: 1049, SEQ ID NO: 1052, SEQ ID NO: 1056, or SEQ ID NO: 1060.

23. The recombinant antibody of claim 1, wherein the antibody comprises a light chain variable region (VL) that comprises a light chain complementarity determining region (CDRL)1, CDRL2, and CDRL3 and a heavy chain variable region (VH) that comprises a heavy chain complementarity determining region (CDRH)1, CDRH2, and CDRH3, wherein:
CDRL1 is SEQ ID NO: 1832;
CDRL2 is SEQ ID NO: 1891;
CRDL3 is SEQ ID NO: 1950;
CRDH1 is SEQ ID NO: 1655;
CRDH2 is SEQ ID NO: 1714; and
CDRH3 is SEQ ID NO: 1773; or
CDRL1 is SEQ ID NO: 1835;
CDRL2 is SEQ ID NO: 1894;
CRDL3 is SEQ ID NO: 1953;
CRDH1 is SEQ ID NO: 1658;
CRDH2 is SEQ ID NO: 1717; and
CDRH3 is SEQ ID NO: 1776; or
CDRL1 is SEQ ID NO: 1849;
CDRL2 is SEQ ID NO: 1908;
CRDL3 is SEQ ID NO: 1967;
CRDH1 is SEQ ID NO: 1672;
CRDH2 is SEQ ID NO: 1731; and
CDRH3 is SEQ ID NO: 1790; or
CDRL1 is SEQ ID NO: 1851;
CDRL2 is SEQ ID NO: 1910;
CRDL3 is SEQ ID NO: 1969;
CRDH1 is SEQ ID NO: 1674;
CRDH2 is SEQ ID NO: 1733; and
CDRH3 is SEQ ID NO: 1792; or
CDRL1 is SEQ ID NO: 1855;
CDRL2 is SEQ ID NO: 1914;
CRDL3 is SEQ ID NO: 1973;
CRDH1 is SEQ ID NO: 1678;
CRDH2 is SEQ ID NO: 1737; and
CDRH3 is SEQ ID NO: 1796; or
CDRL1 is SEQ ID NO: 1860;
CDRL2 is SEQ ID NO: 1919;
CRDL3 is SEQ ID NO: 1978;
CRDH1 is SEQ ID NO: 1683;
CRDH2 is SEQ ID NO: 1742; and
CDRH3 is SEQ ID NO: 1801; or
CDRL1 is SEQ ID NO: 1861;
CDRL2 is SEQ ID NO: 1920;
CRDL3 is SEQ ID NO: 1979;
CRDH1 is SEQ ID NO: 1684;
CRDH2 is SEQ ID NO: 1743; and
CDRH3 is SEQ ID NO: 1802; or
CDRL1 is SEQ ID NO: 1862;
CDRL2 is SEQ ID NO: 1921;
CRDL3 is SEQ ID NO: 1980;
CRDH1 is SEQ ID NO: 1685;
CRDH2 is SEQ ID NO: 1744; and
CDRH3 is SEQ ID NO: 1803; or
CDRL1 is SEQ ID NO: 1864;
CDRL2 is SEQ ID NO: 1923;
CRDL3 is SEQ ID NO: 1982;
CRDH1 is SEQ ID NO: 1687;
CRDH2 is SEQ ID NO: 1746; and
CDRH3 is SEQ ID NO: 1805; or
CDRL1 is SEQ ID NO: 1865;
CDRL2 is SEQ ID NO: 1924;
CRDL3 is SEQ ID NO: 1983;
CRDH1 is SEQ ID NO: 1688;
CRDH2 is SEQ ID NO: 1747; and
CDRH3 is SEQ ID NO: 1806; or
CDRL1 is SEQ ID NO: 1866;
CDRL2 is SEQ ID NO: 1925;
CRDL3 is SEQ ID NO: 1984;
CRDH1 is SEQ ID NO: 1689;
CRDH2 is SEQ ID NO: 1748; and
CDRH3 is SEQ ID NO: 1807; or
CDRL1 is SEQ ID NO: 1872;
CDRL2 is SEQ ID NO: 1931;
CRDL3 is SEQ ID NO: 1990;
CRDH1 is SEQ ID NO: 1695;
CRDH2 is SEQ ID NO: 1754; and
CDRH3 is SEQ ID NO: 1813; or
CDRL1 is SEQ ID NO: 1874;
CDRL2 is SEQ ID NO: 1933;
CRDL3 is SEQ ID NO: 1992;
CRDH1 is SEQ ID NO: 1697;
CRDH2 is SEQ ID NO: 1756; and
CDRH3 is SEQ ID NO: 1815; or
CDRL1 is SEQ ID NO: 1875;
CDRL2 is SEQ ID NO: 1934;
CRDL3 is SEQ ID NO: 1993;
CRDH1 is SEQ ID NO: 1698;
CRDH2 is SEQ ID NO: 1757; and
CDRH3 is SEQ ID NO: 1816; or
CDRL1 is SEQ ID NO: 1878;
CDRL2 is SEQ ID NO: 1937;
CRDL3 is SEQ ID NO: 1996;
CRDH1 is SEQ ID NO: 1701;
CRDH2 is SEQ ID NO: 1760; and
CDRH3 is SEQ ID NO: 1819; or
CDRL1 is SEQ ID NO: 1882;
CDRL2 is SEQ ID NO: 1941;
CRDL3 is SEQ ID NO: 2000;
CRDH1 is SEQ ID NO: 1705;
CRDH2 is SEQ ID NO: 1764; and
CDRH3 is SEQ ID NO: 1823; or
CDRL1 is SEQ ID NO: 1886;
CDRL2 is SEQ ID NO: 1945;
CRDL3 is SEQ ID NO: 2004;
CRDH1 is SEQ ID NO: 1709;
CRDH2 is SEQ ID NO: 1768; and
CDRH3 is SEQ ID NO: 1827.

24.-39. (canceled)

40. A nucleic acid encoding the recombinant antibody of claim 1.

41. (canceled)

42. (canceled)

43. (canceled)

44. A method of treating a coronavirus infection in a subject, comprising administering to the subject a therapeutically effective amount of the recombinant antibody of claim 1.

45. The method of claim 44, where the coronavirus is SARS-CoV-2.
